(19) United States
(12) Patent Application Publication
(10) Pub. No.: US 2025/0223632 A1
(43) Pub. Date: Jul. 10, 2025

(54) PROBE BLOCKERS FOR ENRICHMENT OF TARGET NUCLEIC ACID SEQUENCES

(71) Applicant: Harbinger Health, Inc., Cambridge, MA (US)

(72) Inventor: Anthony P. Shuber, Cambridge, MA (US)

(21) Appl. No.: 19/009,650

(22) Filed: Jan. 3, 2025

Related U.S. Application Data
(60) Provisional application No. 63/618,152, filed on Jan. 5, 2024.

(51) Int. Cl. C12Q 1/6832; C12N 15/10

(52) U.S. Cl. CPC C12Q 1/6832; C12N 15/1006

(57) ABSTRACT

Disclosed are methods for enriching for target nucleic acid sequences by providing small oligonucleotide blocker sequences that hybridize with portions of other nucleic acid sequences (e.g., candidate sequences). The blocker sequences prevent enrichment probes from binding to portions of candidate sequences, thereby preventing subsequent enrichment of candidate sequences. In contrast, enrichment probes bind to target nucleic acid sequences and can be subsequently enriched e.g., via hybrid capture methods.

### CROSS-REFERENCE TO RELATED APPLICATIONS

This application claims the benefit of and priority to U.S. Provisional Patent Application No. 63/618,152 filed Jan. 5, 2024, the entire disclosure of which is hereby incorporated by reference in its entirety for all purposes.

### BACKGROUND

Non-invasive liquid biopsy tests are proving to be the next frontier in cancer diagnostics. There is a rising interest in sensitive, low-cost liquid biopsy assays to detect cancer-specific methylation events. Real-time quantitative methylation-specific PCR (qMSP) allows for the detection of rare methylated fragments, however widespread application has not been achieved. While qMSP achieves high specificity and sensitivity with low cfDNA inputs, assay design is challenged by the mostly three-base genome of bisulfite-converted DNA and the high homology between methylated ctDNA and excessive unmethylated cfDNA background.

### SUMMARY OF THE INVENTION

Disclosed herein are methods for enriching for target nucleic acid sequences. In particular embodiments, target nucleic acid sequences comprise sequences having or corresponding to one or more methylated CpG sites in comparison to other nucleic acids that include sequences, hereafter referred to as candidate sequences, that do not contain or correspond to the same one or more methylated CpG sites. These other nucleic acids can, in various embodiments, only differ from the target nucleic acid sequences due to one or more nucleotides corresponding to the differentially methylated CpG sites. As referred to herein, such other nucleic acids can include a candidate sequence comprising one or more variant sequences e.g., a variant sequence in relation to the target nucleic acid sequence.

Generally, methods for enriching for target nucleic acid sequences involve using small oligonucleotide probes (e.g., >10 bp) to block unmethylated, off-target events. Such small oligonucleotide probes, referred to herein as “blockers,” hybridize with the unmethylated sequences e.g., a variant sequence or a portion thereof. Therefore, probes that are useful for enriching target nucleic acid sequences (e.g., referred to herein as enrichment probes) can selectively hybridize with target nucleic acid sequences as opposed to variant sequences. This enables selective enrichment of target nucleic acid sequences.

Disclosed herein is a method for enriching for a target sequence, the method comprising: obtaining a mixture of nucleic acid sequences, the mixture comprising a first nucleic acid comprising the target sequence and a second nucleic acid comprising a candidate sequence comprising one or more variant sequences; providing an enrichment probe that binds to the target sequence of the first nucleic acid, or a portion thereof; providing a plurality of blocker sequences, each blocker probe comprising a sequence complementary to at least one variant sequence of the candidate sequence; and selectively enriching for the target sequence using the enrichment probe without enriching for the one or more variant sequences of the candidate sequence.

In various embodiments, the target sequence comprises a sequence comprising one or more methylated CpG sites, or a sequence derived from the sequence comprising one or more methylated CpG sites. In various embodiments, the target sequence comprises a sequence comprising at least five consecutive methylated CpG sites, or a sequence derived from the sequence comprising at least five consecutive methylated CpG sites. In various embodiments, the one or more variant sequences of the candidate sequence comprise one or more unmethylated CpG sites. In various embodiments, the one or more variant sequences comprise at least five consecutive non-methylated CpG sites. In various embodiments, each variant sequence of the one or more variant sequences comprises a single unmethylated CpG site. In various embodiments, the candidate sequence comprises five or more variant sequences. In various embodiments, the candidate sequence comprises five or more variant sequences comprising five or more consecutively unmethylated CpG sites.

In various embodiments, the candidate sequence differs from the target sequence by one or more nucleotides based on differential methylation of a corresponding one or more CpG sites. In various embodiments, the enrichment probe is between about 50 nucleotide bases and about 150 nucleotide bases. In various embodiments, the enrichment probe is between about 60 nucleotide bases and about 80 nucleotide bases. In various embodiments, the enrichment probe is about 70 nucleotide bases. In various embodiments, the enrichment probe is between about 80 nucleotide bases and about 120 nucleotide bases. In various embodiments, the enrichment probe is about 100 nucleotide bases. In various embodiments, each of the plurality of blocker sequences are between about 10 and about 20 nucleotide bases in length. In various embodiments, each of the plurality of blocker sequences are between about 15 and about 18 nucleotide bases in length. In various embodiments, each of the plurality of blocker sequences are about 17 nucleotide bases in length.

In various embodiments, the enrichment probe is between about 4 and about 6 times longer in length in comparison to an average length of the plurality of blocker sequences. In various embodiments, the enrichment probe is between about 4 and about 6 times longer in length in comparison to each of the plurality of blocker sequences. In various embodiments, selectively enriching for the target sequence using the enrichment probe without enriching for the one or more variant sequences comprises: hybridizing the enrichment probe to the target sequence, or a portion thereof; hybridizing the plurality of blocker sequences to the one or more variant sequences of the candidate sequence; and enriching for the hybridized enrichment probe while the one or more hybridized blocker probes prevents enrichment for the one or more variant sequences. In various embodiments, enriching for the hybridized enrichment probe while the one or more hybridized blocker probes prevents enrichment for the one or more variant sequences comprises: binding a streptavidin bead to a biotin group of the enrichment probe; and washing and removing unbound nucleic acids, thereby enriching for the target sequence. In various embodiments, the unbound nucleic acids comprise one or more blocker sequences and/or candidate sequences.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings. The drawings are not necessarily to scale, with emphasis instead being placed on illustrating the principles of the present invention, in which:

It is noted that wherever practicable similar or like reference numbers may be used in the figures and may indicate similar or like functionality. For example, a letter after a reference numeral, such as “nucleic acid molecule 415A,” indicates that the text refers specifically to the element having that particular reference numeral. A reference numeral in the text without a following letter, such as “nucleic acid molecule 415” refers to any or all of the elements in the figures bearing that reference numeral (e.g. “nucleic acid molecule 415” in the text refers to reference numerals “nucleic acid molecule 415A” and/or “nucleic acid molecule 415B” in the figures).

FIG. 1 shows an example flow diagram for differentially enriching for nucleic acid molecules, in accordance with an embodiment.

FIG. 2 depicts an example conversion of nucleic acids, in accordance with an embodiment.

FIG. 3 shows the results of nitrite conversion on select nucleotides, in accordance with a second embodiment. Figure adapted from Li et al. (2022) Genome Biology 23:122.

FIG. 4A depict diagrams involving blockers, in accordance with an embodiment.

FIG. 4B depicts a diagram involving provision of probes, in accordance with a first embodiment.

FIG. 5 depicts an overall schematic involving provision of probes (e.g., 70 base or 100 base probes) and/or 17-mer mismatching K5n0 blockers for enrichment of target sequences (e.g., target sequences corresponding to fully methylated sequences).

FIG. 6 shows a percentage of DNA (from a mismatching g-block) isolated by an enrichment probe with and without blockers.

FIG. 7, which shows the percentage of DNA isolated by various probes (e.g., 70-mer and 100-mer probes) with and without blockers

### DETAILED DESCRIPTION

### Definitions

The articles “a” and “an” are used herein to refer to one or to more than one (i.e., to at least one) of the grammatical object of the article. By way of example, “an element” means one element or more than one element.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where a particular value is described in the application and claims, unless otherwise stated the term “about” meaning within an acceptable error range for the particular value can be assumed. The term “about” can have the meaning as commonly understood by one of ordinary skill in the art. The term “about” can refer to ±10%. The term “about” can refer to ±5%.

It should be understood that the expression of “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

As used herein, the term “biological sample,” or “sample” refers to any sample taken from a subject, which can reflect a biological state associated with the subject, and that includes cell free DNA. A biological sample can take any of a variety of forms, such as a liquid biopsy (e.g., blood, urine, stool, saliva, or mucous), or a tissue biopsy, or other solid biopsy. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “nucleic acid” and “nucleic acid molecule” are used interchangeably. The terms refer to nucleic acids of any composition form, such as ribonucleic acid (RNA), deoxyribonucleic acid (DNA, e.g., complementary DNA (cDNA), genomic DNA (gDNA) and the like), and/or RNA/DNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids can comprise protein (e.g., histones, DNA binding proteins, and the like). Nucleic acids analyzed by processes described herein can be substantially isolated and are not substantially associated with protein or other molecules. Nucleic acids can also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides can include deoxyadenosine, deoxycytidine, deoxyguanosine and deoxythymidine. A nucleic acid may be prepared using a nucleic acid obtained from a subject as a template.

As used herein, the terms “template nucleic acid” and “template nucleic acid molecule(s)” are used interchangeably. The terms refer to nucleic acid that has been obtained from a sample and processed to form an immortalized library. The template nucleic acid can be nucleic acid obtained directly from the sample, or nucleic acid that is derived from that obtained directly from the sample. Examples of nucleic acid derived from a sample include DNA that has been reverse-transcribed from RNA obtained directly from a sample, or DNA that has be amplified from DNA obtained directly from a sample, for example, by PCR.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells, or from non-human sources such bacteria, fungi, viruses. Examples of the cell-free nucleic acids include but are not limited to cell-free DNA (“cfDNA”), including mitochondrial DNA or genomic DNA, and cell-free RNA. In certain embodiments herein, instruments for assessing the quality of the cell-free nucleic acids, such as the TapeStation System from Agilent Technologies (Santa Clara, CA) can be used. Concentrating low-abundance cfDNA can be accomplished, for example using a Qubit™ Fluorometer from Thermofisher Scientific (Waltham, MA).

As used herein, the term “methylation” refers to a modification of a nucleic acid where a hydrogen atom on the pyrimidine ring of a cytosine base is converted to a methyl group, forming 5-methylcytosine. Methylation can occur at dinucleotides of cytosine and guanine referred to herein as “CpG sites”. Methylation of cytosine can occur in cytosines in other sequence contexts, for example, 5′-CHG-3′ and 5′-CHH-3′, where His adenine, cytosine or thymine. Cytosine methylation can also be in the form of 5-hydroxymethylcytosine. Methylation of DNA can include methylation of non-cytosine nucleotides, such as N6-methyladenine. Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

Certain portions of a genome comprise regions with a high frequency of CpG sites. A CpG site is portion of a genome that has cytosine and guanine separated by only one phosphate group and is often denoted as “5′-C-phosphate-G-3”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIS and features of the genome are referred to herein as “cancer informative CGIs”, which is defined and described in more detail below. An “informative CpG” can be specified by reference to a specific CpG site, or to a collection of one or more CpG sites by reference to a CG island that contains the collection. These cancer informative CGIs tend to have methylation patterns in tumor cells that are different from the methylation patterns in healthy cells. DNA fragments from other CGIs may not express such differences.

As used herein, “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example, 5′-CHG-3′ and 5′-CHH-3′, where His adenine, cytosine or thymine. Cytosine methylation can also be in the form of 5-hydroxymethylcytosine. Methylation of DNA can include methylation of non-cytosine nucleotides, such as N6-methyladenine.

The phrase “target sequence” refers to a sequence of a nucleic acid derived from a sequence comprising one or more methylated CpG sites. In particular embodiments, a “target sequence” refers to a sequence of a nucleic acid derived from a sequence comprising two, three, four, five, six, seven, eight, nine, ten, eleven, twelve, thirteen, fourteen, fifteen, sixteen, seventeen, eighteen, nineteen, or twenty methylated CpG sites. In various embodiments, such methylated CpG sites are consecutively methylated CpG sites (e.g., no non-methylated CpG sites exist between any two of the consecutively methylated CpG sites). For example, the target sequence may be a sequence of a converted nucleic acid (e.g., where unmethylated cytosines have been converted to uracil and/or where methylated cytosines remain cytosines). In various embodiments, a target sequence includes one or more CpG sites within a region disclosed in Table 1 or Table 2. In particular embodiments, a target sequence includes five CpG sites within a region disclosed in Table 1 or Table 2. In particular embodiments, a target sequence includes five sequential CpG sites within a region disclosed in Table 1 or Table 2.

The phrase “candidate sequence” refers to a sequence of a nucleic acid derived from a sequence comprising one or more non-methylated CpG sites. In particular embodiments, a “candidate sequence” refers to a sequence of a nucleic acid derived from a sequence comprising two, three, four, five, six, seven, eight, nine, ten, eleven, twelve, thirteen, fourteen, fifteen, sixteen, seventeen, eighteen, nineteen, or twenty non-methylated CpG sites (e.g., no methylated CpG sites exist between any two of the consecutively non-methylated CpG sites). In various embodiments, such non-methylated CpG sites are consecutively non-methylated CpG sites. For example, the candidate sequence may be a sequence of a converted nucleic acid (e.g., where unmethylated cytosines have been converted to uracil and/or where methylated cytosines remain cytosines). In various embodiments, a candidate sequence includes one or more CpG sites within a region disclosed in Table 1 or Table 2. In particular embodiments, a candidate sequence includes five CpG sites within a region disclosed in Table 1 or Table 2. In particular embodiments, a candidate sequence includes five sequential CpG sites within a region disclosed in Table 1 or Table 2.

Generally, a “candidate sequence” may differ from a corresponding “target sequence” by one or more nucleotides attributable to differential methylation of the one or more CpG sites. For example, each of a candidate sequence and a corresponding target sequence can include X CpG sites. Assuming that Y CpG sites of the total X CpG sites (where Y is less than X) are differentially methylated between the target sequence and the candidate sequence, then the sequences of the target sequence and the candidate sequence can differ by the Y different nucleotides corresponding to the Y CpG sites. The phrase “candidate sequence” may be referred to herein as including one or more variant sequences of a target sequence.

The phrase “sequential CpG sites” refers to CpG sites within a range of genomic locations in which all CpG sites within the range of genomic locations are part of the sequential CpG sites. Sequential CpG sites include a neighboring CpG site i.e., a previous contiguous or next contiguous CpG site.

The terms “unmethylated” and “non-methylated” are used interchangeably and refer to nucleic acid molecules comprising sequences that include one or more unmethylated CpG sites and/or nucleic acid sequences derived from nucleic acid sequences that include one or more unmethylated CpG sites. For example, “unmethylated nucleic acid molecules” can refer to converted nucleic acid sequences (e.g., bisulfite-converted nucleic acid sequences) that are derived from cell-free DNA that include sequences with one or more unmethylated CpG sites.

As used herein, the term “amplifying” means performing an amplification reaction. In one aspect, an amplification reaction is “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), bisulfite-specific qPCR (qBSP), methylation-specific qPCR (qMSP), linear polymerase reactions, nucleic acid sequence-based amplification (NASBAs), rolling circle amplifications, and the like, disclosed in the following references, each of which are incorporated herein by reference herein in their entirety: Mullis et al., U.S. Pat. Nos. 4,683,195; 4,965,188; 4,683,202; 4,800,159 (PCR); Gelfand et al., U.S. Pat. No. 5,210,015 (real-time PCR with “taqman” probes); Wittwer et al., U.S. Pat. No. 6,174,670; Kacian et al., U.S. Pat. No. 5,399,491 (“NASBA”); Lizardi, U.S. Pat. No. 5,854,033; Aono et al., Japanese patent publ. JP 4-262799 (rolling circle amplification); and the like. As used herein, bisulfite-specific PCR refers to PCR that amplifies converted DNA with no or limited methylation bias. As used herein, methylation-specific PCR refers to PCR that is methylation-specific and bi-sulfite specific, which amplifies converted methylated DNA. In one aspect, the amplification reaction is PCR. An amplification reaction may be a “real-time” amplification if a detection chemistry is available that permits a reaction product to be measured as the amplification reaction progresses, e.g., “real-time PCR”, or “real-time NASBA” as described in Leone et al., Nucleic Acids Research, 26:2150-2155 (1998), and like references.

The terms “fragment” or “segment”, as used interchangeably herein, refer to a portion of a larger polynucleotide molecule. A polynucleotide, for example, can be broken up, or fragmented into, a plurality of segments. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range.

The terms “polymerase chain reaction” or “PCR”, as used interchangeably herein, mean a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors that are well-known to those of ordinary skill in the art, e.g., exemplified by the following references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature>90° C., primers annealed at a temperature in the range 50-75° C., and primers extended at a temperature in the range 72-78° C. The term “PCR” encompasses derivative forms of the reaction, including, but not limited to, RT-PCR, real-time PCR, nested PCR, quantitative PCR, multiplexed PCR, and the like. The particular format of PCR being employed is discernible by one skilled in the art from the context of an application. Reaction volumes can range from a few hundred nanoliters, e.g., 200 nL, to a few hundred μL, e.g., 200 μL. “Reverse transcription PCR,” or “RT-PCR,” means a PCR that is preceded by a reverse transcription reaction that converts a target RNA to a complementary single stranded DNA, which is then amplified, an example of which is described in Tecott et al., U.S. Pat. No. 5,168,038, the disclosure of which is incorporated herein by reference in its entirety. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“taqman”); Wittwer et al., U.S. Pat. Nos. 6,174,670 and 6,569,627 (intercalating dyes); Tyagi et al., U.S. Pat. No. 5,925,517 (molecular beacons); the disclosures of which are hereby incorporated by reference herein in their entireties. Detection chemistries for real-time PCR are reviewed in Mackay et al., Nucleic Acids Research, 30:1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g., Bernard et al., Anal. Biochem., 273:221-228 (1999) (two-color real-time PCR). Usually, distinct sets of primers are employed for each sequence being amplified. Typically, the number of target sequences in a multiplex PCR is in the range of from 2 to 50, or from 2 to 40, or from 2 to 30. In particular embodiments, the number of target sequences in a multiplex PCR is about 4. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β2-microglobulin, ribosomal RNA, and the like. Techniques for quantitative PCR are well-known to those of ordinary skill in the art, as exemplified in the following references, which are incorporated by reference herein in their entireties: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); and Becker-Andre et al., Nucleic Acids Research, 17:9437-9446 (1989).

The term “primer” as used herein means an oligonucleotide, either natural or synthetic, that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2nd Edition (Cold Spring Harbor Press, New York, 2003).

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a women or a child).

As used herein “selective enrichment of nucleic acid molecules” refers to the increased enrichment of nucleic acid molecules in relation to other nucleic acids. In various embodiments, selective enrichment refers to at least a fold enrichment of nucleic acid molecules relative to other nucleic acid molecules. Thus, selective enrichment of nucleic acid molecules does not require complete abatement or elimination of other nucleic acid molecules. Rather, at least a fold increase in the amplification and/or hybrid capture of nucleic acid molecules is achieved in comparison to other nucleic acid molecules. In various embodiments, selective enrichment of nucleic acid molecules refers to at least a 2-fold increase, at least a 3-fold increase, at least a 4-fold increase, at least a 5-fold increase, at least a 6-fold increase, at least a 7-fold increase, at least a 8-fold increase, at least a 9-fold increase, at least a 10-fold increase, at least a 15-fold increase, at least a 20-fold increase, at least a 25 fold increase, at least a 50-fold increase, at least a 100-fold increase, at least a 200-fold increase, at least a 500-fold increase, or at least a 1000-fold increase of the nucleic acid molecules relative to other nucleic acids.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

Any example(s) following the term “e.g.” or “for example” is not meant to be exhaustive or limiting.

Unless otherwise required by context, singular terms shall include pluralities and plural terms shall include the singular.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to be inclusive of the numbers defining the range and to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Unless otherwise defined herein, scientific and technical terms used in this application shall have the meanings that are commonly understood by those of ordinary skill in the art.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in an embodiment of the disclosure.

Exemplary methods and materials are described herein, although methods and materials similar or equivalent to those described herein can also be used in the practice or testing of the present disclosure. The materials, methods, and examples are illustrative only and not intended to be limiting.

### Overview

Disclosed herein are methods for enriching nucleic acid molecules comprising a target sequence. Such methods are useful for enriching for a signal in a sample, such as a signal informative for determining presence or absence of cancer in the sample. In various embodiments, methods disclosed herein are useful for enriching for nucleic acids including sequences derived from methylated CpG sites by inhibiting or blocking other sequences derived from non-methylated CpG sites. For example, this enrichment technology can be used for methylation detection from bisulfite-converted DNA in qPCR, dPCR, hybridization capture, etc. Methods include providing short blocker sequences that bind to candidate sequences or portions thereof (e.g., a variant sequence), thereby inhibiting longer enrichment probe sequences from hybridizing with candidate sequences. This prevents the downstream processing and capturing of candidate sequences. In contrast, the short blocker sequences do not bind, or exhibit reduced affinity for target sequences, thereby allowing for longer probe sequences to hybridize with target sequences. This enables the downstream processing, capturing, and enrichment of target sequences.

In various embodiments, methods for enriching a target sequence comprises: obtaining a mixture of nucleic acid sequences, the mixture comprising a first nucleic acid comprising the target sequence and a second nucleic acid comprising a candidate sequence comprising one or more variant sequences; providing an enrichment probe that binds to the target sequence of the first nucleic acid, or a portion thereof; providing a plurality of blocker sequences, each blocker probe comprising a sequence complementary to at least one variant sequence of the candidate sequence; and selectively enriching for the target sequence using the enrichment probe without enriching for the one or more variant sequences of the candidate sequence. Generally, a blocker sequence of the plurality of blocker sequences may contain a sequence that is at least 90%, at least 95%, or 100% complementary to the candidate sequence, or a portion thereof (e.g., a variant sequence). Given that the first nucleic acid molecule was derived from a sequence comprising one or more methylated CpG sites, the plurality of blocker sequences each comprise a sequence that contains one or more mismatches relative to the target sequence, or a portion thereof. Thus, the blocker does not bind to the first nucleic acid (or binds to the first nucleic acid at a rate that is less than a rate at which the blocker binds to the candidate sequence, or portion thereof).

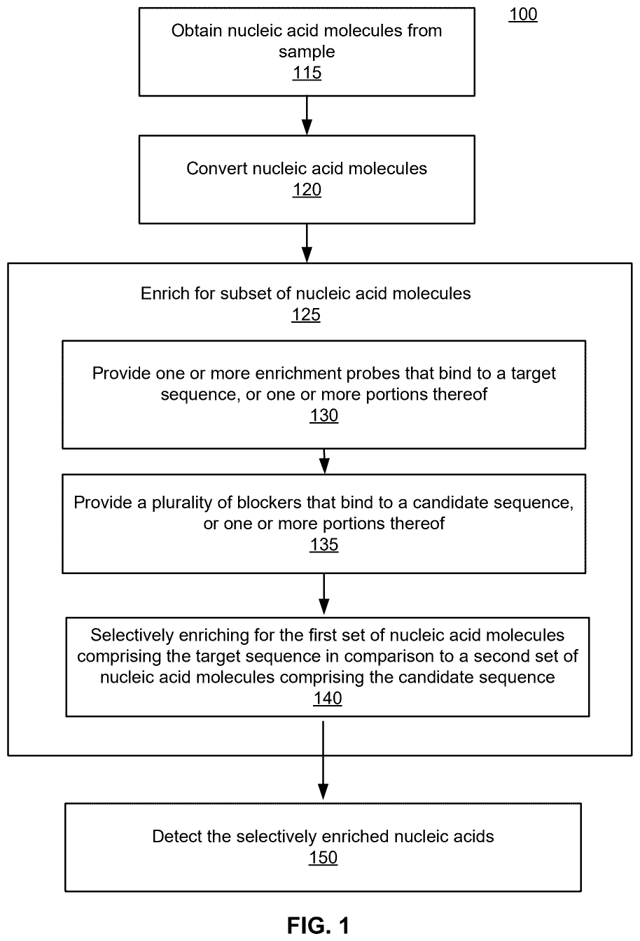

Reference is now made to FIG. 1, which shows an example flow diagram 100 for differentially enriching for nucleic acid molecules, in accordance with an embodiment. As shown in FIG. 1, step 115 involves obtaining a sample (e.g., a sample from a subject). In various embodiments, a sample is any of a tissue sample, a blood sample, a stool sample, a urine sample, a mucous sample, or a saliva sample. In particular embodiments, a sample is a blood sample or a serum sample. In particular embodiments, a sample is a tissue sample. The sample can be obtained by the subject or by a third party, e.g., a medical professional. Examples of medical professionals include physicians, emergency medical technicians, nurses, first responders, psychologists, phlebotomists, medical physics personnel, nurse practitioners, surgeons, dentists, and any other medical professional as would be known to one skilled in the art. In various embodiments, the one or more samples can be obtained from the subject by a reference lab.

In various embodiments, the sample obtained from the subject is a liquid biopsy sample. In various embodiments, the liquid biopsy sample includes nucleic acid molecules. Example nucleic acid molecules include DNA or RNA. In particular embodiments, the nucleic acid molecules include cell-free DNA (cfDNA). In various embodiments, the cfDNA includes genomic sequences corresponding to CpG islands (CGIs) for which methylation states are informative for presence or absence of cancer. In various embodiments, the cfDNA can be derived from tumor cells and is referred to herein as circulating tumor DNA (ctDNA). In various embodiments, the nucleic acid molecules include a mixture of nucleic acid molecules that contain either methylated CpG sites or non-methylated CpG sites. For example, for a particular genomic region containing one or more CpG sites, the mixture of nucleic acid molecules includes a subset of nucleic acid molecules in which the one or more CpG sites are unmethylated and in a different subset of nucleic acid molecules in which the one or more CpG sites are partially or fully methylated.

Step 120 involves converting the nucleic acid molecules from the sample obtained from the subject. In various embodiments, converting the nucleic acid involves converting unmethylated nucleotides (e.g., cytosines) to another nucleotide (a “converted nucleotide”, as used herein). In various embodiments, methylated cytosines are protected from conversion (e.g., deamination) during the conversion step. Further details of performing conversion of nucleic acid molecules (e.g., step 120) are described herein.

Although not shown in FIG. 1, in various embodiments, after conversion of nucleic acids, the converted nucleic acids undergo library construction. In various embodiments, converted nucleic acids can undergo end-repairing, tailing of 3′ ends, and/or addition of library or sequencing adapters. In various embodiments, converted nucleic acids can undergo biotinylation (e.g., addition of biotin moieties to converted nucleic acids). In various embodiments, barcodes can be incorporated into converted nucleic acids, thereby enabling subsequent sample demultiplexing (e.g., demultiplexing to identify sources of converted nucleic acids or demultiplexing to identify a common source from converted nucleic acids). In various embodiments, one or more washes and/or selections can be performed to remove unwanted DNA fragments, such as single stranded DNA fragments, excess adapters, and other molecules. In particular embodiments, a solid-phase reversible immobilization (SPRI) selection is performed. As used herein, a “nucleic acid template” refers to a nucleic acid derived from the converted nucleic acid (e.g., any of a nucleic acid derived from a converted nucleic acid that underwent library construction, end-repairing, addition of library or sequencing adapters, barcode addition, or any combination thereof).

Step 125 involves enriching for a subset of nucleic acid molecules (e.g., nucleic acid molecules comprising target sequences). As shown in FIG. 1, step 125 can include substeps 130, 135, and 140. Generally, enriching for a subset of nucleic acid molecules comprises providing one or more blocker sequences. The blocker sequences bind to candidate sequences, or portions thereof (e.g., a variant sequence) of converted nucleic acid molecules, the candidate sequences derived from sequences comprising one or more non-methylated CpG sites. For example, a candidate sequence may contain nucleotides that were converted from corresponding non-methylated CpG sites and/or subsequently amplified. Thus, following conversion, a non-methylated CpG site would be converted to a “UG” sequence. Subsequent amplification may generate a corresponding “TG” sequence that is present in the candidate sequence. By binding candidate sequences with blocker sequences, the candidate sequences cannot undergo subsequent processing, thereby allowing for enrichment of target nucleic acid sequences.

Although steps 130, 135, and 140 are shown in FIG. 1 in a particular order, in various embodiments, the ordering of one or more of steps 130, 135, and 140 may differ. In some embodiments, step 130 (providing one or more enrichment probes) occurs after step 135 (providing a plurality of blockers). In some embodiments, steps 130 and 135 occur simultaneously (e.g., both the enrichment probe and the plurality of blockers are provided together).

Step 130 involves providing one or more enrichment probes that bind to a target sequence, or one or more portions thereof. In various embodiments, an enrichment probe comprises a sequence that is at least 90% complementary, at least 95% complementary, at least 96% complementary, at least 97% complementary, at least 98% complementary, at least 99% complementary, or 100% complementary to the target sequence, or a portion thereof.

In various embodiments, a target sequence contains a sequence that includes or corresponds to X CpG sites that were partially or fully methylated. In particular embodiments, a target sequence contains a sequence that includes or corresponds to X CpG sites that were fully methylated. Thus, by binding the enrichment probe to the target sequence, or a portion thereof, the subsequent processing and enrichment steps can enrich for the target sequence using the enrichment probe.

Step 135 involves providing a plurality of blockers that bind to a candidate sequence, or portions thereof (e.g., a variant sequence). In various embodiments, step 135 involves providing copies of 1 blocker sequence, 2 blocker sequences, 3 blocker sequences, 4 blocker sequences, 5 blocker sequences, 6 blocker sequences, 7 blocker sequences, 8 blocker sequences, 9 blocker sequences, 10 blocker sequences, 11 blocker sequences, 12 blocker sequences, 13 blocker sequences, 14 blocker sequences, 15 blocker sequences, 16 blocker sequences, 17 blocker sequences, 18 blocker sequences, 19 blocker sequences, or 20 blocker sequences. In particular embodiments, the plurality of blocker sequences comprise 5 blocker sequences that are complementary to 5 different variant sequences of a candidate sequence. Each blocker sequence can be complementary to a single variant sequence of the candidate sequence.

Returning to FIG. 1, step 140 involves selectively enriching for a first set of nucleic acid molecules comprising a target sequence (e.g., target sequence derived from one or more methylated CpG sites). Here, the first of nucleic acid molecules are selectively enriched in comparison to a second set of nucleic acid molecules comprising a candidate sequence (e.g., candidate sequence derived from one or more non-methylated CpG sites). Here, the presence of the plurality of blockers bound to a candidate sequence prevents or reduces enrichment of the second set of nucleic acid molecules comprising the candidate sequence.

In various embodiments, the probe is complementary to a portion of the candidate sequence that is bound by the blocker. For example, the probe may be complementary to at least one nucleotide, at least two nucleotides, at least three nucleotides, at least four nucleotides, at least five nucleotides, at least six nucleotides, at least seven nucleotides, at least eight nucleotides, at least nine nucleotides, at least ten nucleotides, at least eleven nucleotides, at least twelve nucleotides, at least thirteen nucleotides, at least fourteen nucleotides, at least fifteen nucleotides, at least sixteen nucleotides, at least seventeen nucleotides, at least eighteen nucleotides, at least nineteen nucleotides, or at least twenty nucleotides that are bound by one or more blockers. In various embodiments, the one or more blockers outcompete the enrichment probe for binding to the candidate sequence, or portion thereof. For example, the plurality of blockers can each be bound to a different portion of the candidate sequence, thereby preventing the enrichment probe from hybridizing to the candidate sequence. As another example, the plurality of blockers can outcompete binding of the enrichment probe. Thus, when the subsequent enrichment step is performed, the candidate sequence is not enriched.

In various embodiments, step 140, involves performing an enrichment step. An example enrichment step includes performing any one of hybrid capture, use of DNA-binding proteins to enrich a target sequence or a subset of target sequences, and nucleic acid amplification (e.g., polymerase chain reaction). In particular embodiments, the enrichment step involves capturing target nucleic acids on a solid support through an affinity moiety. For example, the enrichment step can involve capturing target nucleic acids through a biotin-streptavidin interaction, where the enrichment probe includes a biotin group and a solid support is coated with streptavidin groups. Therefore, the target nucleic acid is captured by the solid support through the biotin-streptavidin interaction.

In various embodiments, step 140 involves performing one or more washes and/or selections to remove unwanted DNA fragments, such as single stranded DNA fragments, excess primers, excess adapters, and other molecules. For example, nucleic acids that remain unbound (e.g., not bound to a streptavidin coated solid support) are washed and removed, while retaining bound target nucleic acids. Thus, the bound target nucleic acids are enriched relative to the unbound nucleic acids.

Step 150 involves detecting the selectively enriched nucleic acids. In various embodiments, detecting the selectively enriched nucleic acids involves performing sequencing to determine the sequences of the first set of nucleic acids. In various embodiments, sequencing data can be demultiplexed e.g., using barcode sequences. In various embodiments, sequencing data can be aligned to a reference genome and/or trimmed. In various embodiments, sequencing data can be further analyzed to determine the enriched signal in the sample e.g., for determining presence or absence of cancer in the sample. In various embodiments, detecting the selectively enriched nucleic acids involves quantifying a signal from the first set of nucleic acids. For example, the signal may be a fluorescent signal. Thus, quantifying the fluorescent signal can be informative for determining a total quantity of the first set of nucleic acid molecules (or nucleic acids, such as amplicons, derived from the first set of nucleic acid molecules. Detecting the selectively enriched nucleic acids by quantifying a signal from the first set of nucleic acids can be performed e.g., when performing quantitative PCR.

### Methods for Enriching Target Nucleic Acid Sequences

a. Exemplary Methods

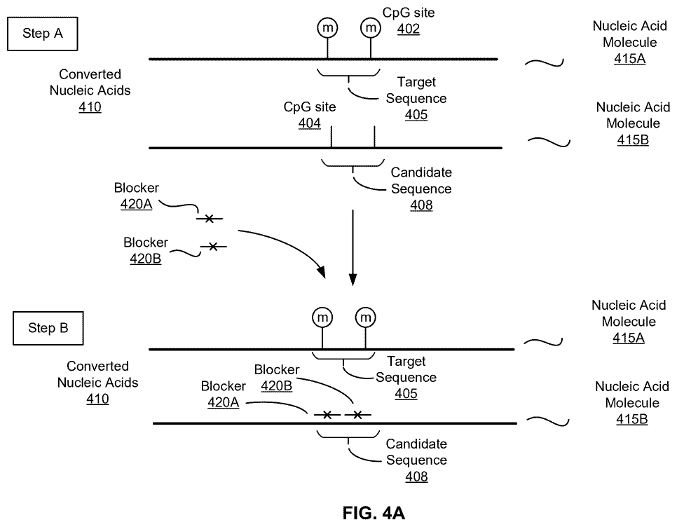

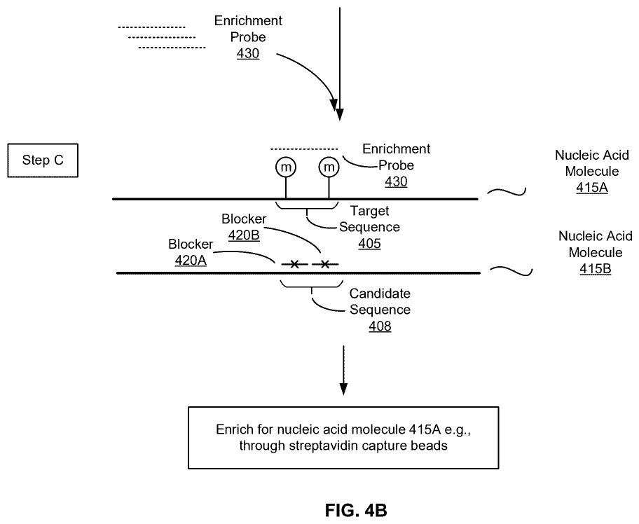

As disclosed herein in reference to FIG. 1, methods can involve step 125 for enriching a subset of nucleic acid molecules e.g., nucleic acid molecules comprising target sequences. Methods for enriching the subset of nucleic acid molecules is further described in reference to FIGS. 4A-4B, which depict diagrams involving provision of enrichment probes and blockers, in accordance with various embodiments.

Beginning with FIG. 4A, it depicts a diagram involving blockers, in accordance with an embodiment. Step A begins with converted nucleic acids 410, including nucleic acid molecule 415A and nucleic acid molecule 415B. Step A shows the converted nucleic acids 410 after having performed step 120 shown in FIG. 1. Here, nucleic acid molecule 415A may include a target sequence 405 that is derived from a sequence comprising one or more methylated CpG sites. FIG. 4A shows two genomic sites (labeled as “m”) that are derived from corresponding CpG sites 402 that were previously methylated. For example, the prior CpG sites may have a sequence of “CG” where the cytosine was methylated. Following conversion, the “CG” remains and the target sequence 405 can include a “CG”, or complement thereof, at each site corresponding to a CpG site. Although FIG. 4A explicitly shows a target sequence 405 including two CpG sites, in various embodiments, a target sequence can include additional CpG sites (e.g., 3, 4, 5, 6, 7, 8, 9, 10, 11, 12, 13, 14, 15, 16, 17, 18, 19, or 20 CpG sites). In particular embodiments, a target sequence includes 5 CpG sites.

Additionally, nucleic acid molecule 415B includes a candidate sequence 408 that is derived from a sequence comprising one or more non-methylated CpG sites. Specifically, FIG. 4A shows two genomic sites that are derived from corresponding CpG sites 404 that were previously unmethylated. For example, the prior CpG sites may have a sequence of “CG” where the cytosine was non-methylated. Following conversion, the “CG” is converted to “UG”. Thus, the candidate sequence 408 can include a “TG”, or complement thereof, at each site corresponding to a CpG site. Although FIG. 4A explicitly shows a candidate sequence 408 including two CpG sites, in various embodiments, a candidate sequence can include additional CpG sites (e.g., 3, 4, 5, 6, 7, 8, 9, 10, 11, 12, 13, 14, 15, 16, 17, 18, 19, or 20 CpG sites). In particular embodiments, a candidate sequence includes 5 CpG sites.

One or more blockers (e.g., blocker 420A and blocker 420B) are introduced to the converted nucleic acids 410. The one or more blockers 420 bind to portions of candidate sequence 408 of nucleic acid molecule 415B. Here, each of blocker 420A and blocker 420B bind to a portion of candidate sequence 408. Although FIG. 4A shows an embodiment include provision of two blocker sequences e.g., blocker 420A and blocker 420B, in some embodiments, copies of additional blocker sequences can be provided (e.g., 3 blocker sequences, 4 blocker sequences, 5 blocker sequences, 6 blocker sequences, 7 blocker sequences, 8 blocker sequences, 9 blocker sequences, 10 blocker sequences, 11 blocker sequences, 12 blocker sequences, 13 blocker sequences, 14 blocker sequences, 15 blocker sequences, 16 blocker sequences, 17 blocker sequences, 18 blocker sequences, 19 blocker sequences, or 20 blocker sequences).

In various embodiments, the provided number of different blocker sequences is dependent on the number of CpG sites in the candidate sequence 408. For example, if the candidate sequence 408 includes X total CpG sites, then X different blocker sequences can be provided. In such embodiments, each blocker sequence can comprise a sequence that is complementary to a variant sequence of the candidate sequence 408, where each variant sequence of the candidate sequence 408 includes a single CpG site of the X total CpG sites. Thus, every CpG site in a variant sequence of the candidate sequence 408 can be bound by at least a corresponding blocker sequence. As an example, FIG. 4A shows a blocker 420A and a blocker 420B each bound to a portion of candidate sequence 408. Here, blocker 420A may include a sequence complementary to first variant sequence of the candidate sequence 408, the first variant sequence including a first CpG site. Additionally, blocker 420B may include a sequence complementary to second variant sequence of the candidate sequence 408, the second variant sequence including a second CpG site. Together, blocker 420A and blocker 420B bind to the different variant sequences of the candidate sequence 408, thereby preventing a subsequent probe from binding to the candidate sequence 408.

In various embodiments, if the candidate sequence 408 includes X total CpG sites, then fewer than X different blocker sequences can be provided. Here, each of the X different blocker sequences can bind to at least one of the X total CpG sites. In some scenarios, at least one of the blocker sequences can bind to two or more of the X total CpG sites. Therefore, fewer than X different blocker sequences can be provided while still binding to the X total CpG sites. This may be beneficial in situations where adjacent CpG sites are located near to each other such that a single blocker sequence can include a sequence that is complementary to a variant sequence comprising the adjacent CpG sites. In various embodiments, two CpG sites are located near each other if they are within 50 nucleotides, within 40 nucleotides, within 30 nucleotides, within 25 nucleotides, within 20 nucleotides, within 19 nucleotides, within 18 nucleotides, within 17 nucleotides, within 16 nucleotides, within 15 nucleotides, within 14 nucleotides, within 13 nucleotides, within 12 nucleotides, within 11 nucleotides, within 10 nucleotides, within 9 nucleotides, within 8 nucleotides, within 7 nucleotides, within 6 nucleotides, within 5 nucleotides, within 4 nucleotides, within 3 nucleotides, within 2 nucleotides, or within 1 nucleotide of each other. As an example, X−1 different blocker sequences can be provided where a blocker sequence binds to a variant sequence comprising 2 CpG sites. The remaining blocker sequences can each bind to one of the remaining CpG sites.

In various embodiments, two blockers 420 are designed such that when each blocker is hybridized with a portion of the candidate sequence 408, the two blockers 420 are immediately adjacent to each other. As an example, a first blocker may bind to a variant sequence of the candidate sequence 408 that is immediately adjacent to another variant sequence of the candidate sequence 408 that is bound by a second blocker. As shown in FIG. 4A, blocker 420A may bind to a first variant sequence of candidate sequence 408 that is immediately adjacent (e.g., immediately upstream) to a second variant sequence of candidate sequence 408. As used herein, “immediately adjacent” sequences refer to two sequences in which no nucleotides are located between the two sequences.

In various embodiments, two blockers 420 are designed such that when each blocker is hybridized with a portion of the candidate sequence 408, the two blockers 420 are bound to two different variant sequences of the candidate sequence 408 that are separated by 1 or more nucleotides. In various embodiments, the two blockers 420 are bound to two different variant sequences of the candidate sequence 408 that are separated by 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 90 nucleotides, or 100 nucleotides. In various embodiments, the two blockers 420 are bound to two different variant sequences of the candidate sequence 408 that are separated by between 100 and 200 nucleotides, between 200 and 300 nucleotides, between 300 and 400 nucleotides, between 400 and 500 nucleotides, between 500 and 600 nucleotides, between 600 and 700 nucleotides, between 700 and 800 nucleotides, between 800 and 900 nucleotides, between 900 and 1000 nucleotides, or between 1000 and 5000 nucleotides.

In various embodiments, the plurality of blockers 420 are designed such that when each blocker is hybridized with a portion of the candidate sequence 408, the entire candidate sequence 408 is bound by one of the plurality of blockers 420. In various embodiments, when the plurality of blockers 420 are each hybridized with a portion of the candidate sequence 408, there may be other portions of the candidate sequence 408 that remain unbound. In such embodiments, the plurality of blockers 420 may bind the variant sequences (e.g., variant sequences including CpG sites) but need not bind all of the other portions of the candidate sequence 408. Generally, the plurality of blockers 420 bind to a sufficient proportion of the candidate sequence 408 such that the plurality of blockers 420 prevents binding of a subsequent enrichment probe to the candidate sequence 408.

In various embodiments, when the plurality of blockers 420 are each hybridized with a portion of the candidate sequence 408, between 8% and 50% of the candidate sequence 408 is bound. In various embodiments, when the plurality of blockers 420 are each hybridized with a portion of the candidate sequence 408, between 10% and 30% of the candidate sequence 408 is bound. In various embodiments, when the plurality of blockers 420 are each hybridized with a portion of the candidate sequence 408, between 15% and 25% of the candidate sequence 408 is bound. In various embodiments, when the plurality of blockers 420 are each hybridized with a portion of the candidate sequence 408, between 18% and 22% of the candidate sequence 408 is bound. In various embodiments, when the plurality of blockers 420 are each hybridized with a portion of the candidate sequence 408, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the candidate sequence 408 is bound.

In various embodiments, a blocker 420 does not bind, or minimally binds, to target sequence 405. Given the mismatches between the sequence of the plurality of blockers 420 and the nucleotides of the target sequence 405 that correspond to methylated CpG sites 402, the blocker 420 may fail to hybridize, or hybridizes to a lesser extent, with the target sequence 405. In various embodiments, less than 50% of nucleic acid molecule 415A (e.g., first set of nucleic acid molecules that are derived from sequences with one or more methylated CpG sites) are bound to a blocker 420. In various embodiments, less than 40%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1% of nucleic acid molecule 415A are bound to a blocker 420.

Next, enrichment probes are provided. Reference is made to FIG. 4B, which depicts a diagram involving provision of enrichment probes 430, in accordance with an embodiment. The enrichment probe 430 is capable of hybridizing to the target sequence 405, or a portion thereof, of the nucleic acid molecule 415A. In various embodiments, the enrichment probe 430 comprises a sequence that is at least 90% complementary, at least 95% complementary, at least 96% complementary, at least 97% complementary, at least 98% complementary, at least 99% complementary, or 100% complementary to the target sequence, or a portion thereof. In various embodiments, the enrichment probe 430 is also capable of hybridizing to the candidate sequence 408, albeit to a lesser extent than hybridizing to the target sequence 405. However, the presence of the blockers 420 bound to the candidate sequence 408 prevents or reduces the binding of the enrichment probe 430 to the candidate sequence 408. In various embodiments, less than 40%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1% of nucleic acid molecule 415B are bound to an enrichment probe 430.

Given that the enrichment probe 430 is bound to nucleic acid molecule 415A and not bound to nucleic acid molecule 415B, subsequent enrichment can be performed to enrich for nucleic acid molecule 415A using the bound enrichment probe 430. As an example, methods can involve performing hybrid capture e.g., using a solid support to capture the enrichment probe 430 bound to the target sequence 405 of the nucleic acid molecule 415A. As another example, methods can involve performing capturing the enrichment probe 430 and nucleic acid molecule 415A via an affinity moiety (e.g., a biotin and/or streptavidin) using a solid support. Thus, the nucleic acid molecule 415A can be enriched while eliminating or reducing the quantity of nucleic acid molecule 415B.

b. Example Blocker Sequences and Enrichment Probe Sequences

As disclosed herein, a blocker may contain a sequence that is at least 90%, at least 95%, or 100% complementary to one or more variant sequences of a candidate sequence, or a portion thereof. In various embodiments, a blocker may contain a sequence that is at least 90%, at least 95%, or 100% complementary to one or more variant sequences of a candidate sequence, or a portion thereof. In various embodiments, a blocker may be designed to be at least 90%, at least 95%, or 100% complementary to one or more variant sequences of a candidate sequence that includes one or more CpG sites within a region disclosed in Table 1 or Table 2. In various embodiments, a blocker may be designed to be at least 90%, at least 95%, or 100% complementary to one or more variant sequences of a candidate sequence that include two or more, three or more, four or more, five or more, six or more, seven or more, eight or more, nine or more, or ten or more CpG sites within a region disclosed in Table 1 or Table 2. In various embodiments, a blocker may be designed to be at least 90%, at least 95%, or 100% complementary to one or more variant sequences of a candidate sequence that includes two, three, four, five, six, seven, eight, nine, or ten CpG sites within a region disclosed in Table 1 or Table 2. In various embodiments, a blocker may be designed to be at least 90%, at least 95%, or 100% complementary to one or more variant sequences of a candidate sequence that includes five sequential CpG sites within a region disclosed in Table 1 or Table 2. In various embodiments, a blocker is designed to be 100% complementary to one or more variant sequences of a candidate sequence that includes five sequential CpG sites within a region disclosed in Table 1 or Table 2.

In various embodiments, a blocker may be designed to be complementary to one or more variant sequences of a candidate sequence, the candidate sequence containing X CpG sites, where the X CpG sites are fully unmethylated. In various embodiments, a blocker may be designed to be complementary to one or more variant sequences of a candidate sequence that contains X CpG sites, where at least 10%, at least 20%, at least 30%, at least 40%, at least 50%, at least 60%, at least 70%, at least 80%, or at least 90% of the X CpG sites are unmethylated.

As referred to herein, the term “K*n #” refers to a sequence having “*” CpG sites, where “#” of the CpG sites that are methylated. Therefore, the term “K5n5” refers to a sequence including 5 CpG sites in which 5 of the CpG sites are methylated. As another example, the term “K6n5” refers to a sequence including 6 CpG sites in which 5 of the CpG sites are methylated. In various embodiments, “*” can be any of 1, 2, 3, 4, 5, 6, 7, 8, 9, or 10. In various embodiments, “#” can be any of 1, 2, 3, 4, 5, 6, 7, 8, 9, or 10. In some embodiments, a candidate sequence is less than 200 bp, less than 150 bp, less than 100 bp, less than 75 bp, less than 50 bp in length, or less than 35 bp in length. In some embodiments, a candidate sequences is less than 150 bp in length. In some embodiments, a candidate sequences is less than 100 bp in length. In some embodiments, a candidate sequences is less than 75 bp in length. In some embodiments, a candidate sequences is less than 50 bp in length. In some embodiments, a candidate sequence is less than 35 bp in length.

In various embodiments, a blocker may be designed to be fully complementary to one or more variant sequences of a candidate sequence derived from a “K*n #” sequence. For example, a blocker may be designed to be complementary to one or more variant sequences of a candidate sequence derived from a K5n0 sequence (e.g., fully unmethylated sequence of 5 CpG sites). Thus, as each of the CpG sites of the K5n0 sequence is unmethylated, the resulting sequence corresponding to each CpG site may be “TG”, or a complement thereof. Thus, a blocker may be designed to have a sequence that is complementary to a variant sequence including one of the five “TG” nucleotides, or a complement thereof.

In various embodiments, a blocker may be designed to be complementary to one or more variant sequences of a candidate sequence derived from any of a K1n0 sequence, a K2n0 sequence, a K3n0 sequence, a K4n0 sequence, a K5n0 sequence, a K6n0 sequence, a K7n0 sequence, a K8n0 sequence, a K9n0 sequence, or a K10n0 sequence. In various embodiments, a blocker may be designed to be complementary to one or more variant sequences of a candidate sequence derived from any of a K2n1 sequence, a K3n1 sequence, a K4n1 sequence, a K5n1 sequence, a K6n1 sequence, a K7n1 sequence, a K8n1 sequence, a K9n1 sequence, a K10n1 sequence, a K3n2 sequence, a K4n2 sequence, a K5n2 sequence, a K6n2 sequence, a K7n2 sequence, a K8n2 sequence, a K9n2 sequence, a K10n2 sequence, a K4n3 sequence, a K5n3 sequence, a K6n3 sequence, a K7n3 sequence, a K8n3 sequence, a K9n3 sequence, a K10n3 sequence, a K5n4 sequence, a K6n4 sequence, a K7n4 sequence, a K8n4 sequence, a K9n4 sequence, a K10n4 sequence, a K6n5 sequence, a K7n5 sequence, a K8n5 sequence, a K9n5 sequence, a K10n5 sequence, a K7n6 sequence, a K8n6 sequence, a K9n6 sequence, a K10n6 sequence, a K8n7 sequence, a K9n7 sequence, a K10n7 sequence, a K9n8 sequence, a K10n8 sequence, or a K10n9 sequence.

In various embodiments, a blocker is designed to be complementary to a “K*n0” variant sequence, where “*” is between 1 and 5. In particular embodiments, a blocker is designed to be complementary to a K1n0 variant sequence (e.g., a variant sequence including 1 CpG site that was previously unmethylated). In various embodiments, a blocker is designed to be complementary to a K2n0 variant sequence (e.g., a variant sequence including 2 CpG sites that were each previously unmethylated), a K3n0 variant sequence (e.g., a variant sequence including 3 CpG sites that were each previously unmethylated), a K4n0 variant sequence (e.g., a variant sequence including 4 CpG sites that were each previously unmethylated), or a K5n0 variant sequence (e.g., a variant sequence including 5 CpG sites that were each previously unmethylated).

In particular embodiments, a candidate sequence is a K5n0 sequence and the plurality of blockers include 5 different blockers, each designed to be complementary to a particular K1n0 variant sequence. Each blocker may be designed to bind to a different K1n0 variant sequence, thereby covering all CpG sites in the K5n0 candidate sequence.

In various embodiments, a blocker may include a sequence that is between 1 and 40 nucleotide bases in length. In various embodiments, a blocker may include a sequence that is between 5 and 30 nucleotide bases in length, between 10 and 20 nucleotide bases in length, between 13 and 19 nucleotide bases in length, or between 15 and 18 nucleotide bases in length. In various embodiments, a blocker may include a sequence that is 17 nucleotide bases in length.

As disclosed herein, methods may include providing a plurality of blocker sequences (e.g., see FIG. 4A). In various embodiments, blocker sequences of the plurality of blocker sequences may each have the same length. As one example, each blocker sequence of the plurality of blocker sequences may have a length between 1 and 40 nucleotide bases, between 5 and 30 nucleotide bases, between 10 and 20 nucleotide bases, between 13 and 19 nucleotide bases, or between 15 and 18 nucleotide bases. As an example, each blocker sequence of the plurality of blocker sequences may have a length of 17 nucleotide bases.

As disclosed herein, an enrichment probe may contain a sequence that is at least 90% complementary, at least 95% complementary, at least 96% complementary, at least 97% complementary, at least 98% complementary, at least 99% complementary, or 100% complementary to the target sequence, or a portion thereof. In various embodiments, an enrichment probe may be designed to be at least 90%, at least 95%, or 100% complementary to a target sequence that includes one or more CpG sites within a region disclosed in Table 1 or Table 2. In various embodiments, an enrichment probe may be designed to be at least 90%, at least 95%, or 100% complementary to a target sequence that includes two or more, three or more, four or more, five or more, six or more, seven or more, eight or more, nine or more, or ten or more CpG sites within a region disclosed in Table 1 or Table 2. In various embodiments, an enrichment probe may be designed to be at least 90%, at least 95%, or 100% complementary to a target sequence that includes two, three, four, five, six, seven, eight, nine, or ten CpG sites within a region disclosed in Table 1 or Table 2. In various embodiments, an enrichment probe may be designed to be at least 90%, at least 95%, or 100% complementary to a target sequence that includes five sequential CpG sites within a region disclosed in Table 1 or Table 2. In various embodiments, an enrichment probe is designed to be 100% complementary to a target sequence that includes five sequential CpG sites within a region disclosed in Table 1 or Table 2.

In various embodiments, an enrichment probe may be designed to be complementary to a target sequence that contains X CpG sites, where the X CpG sites are fully methylated. In various embodiments, an enrichment probe may be designed to be complementary to a target sequence that contains X CpG sites, where the X CpG sites are fully methylated. In various embodiments, an enrichment probe may be designed to be complementary to a target sequence that contains X CpG sites, where at least 10%, at least 20%, at least 30%, at least 40%, at least 50%, at least 60%, at least 70%, at least 80%, or at least 90% of the X CpG sites are methylated.

In various embodiments, an enrichment probe may be designed to be fully complementary to a target sequence derived from a “K*n #” sequence. For example, an enrichment probe may be designed to be complementary to a target sequence derived from a K5n5 sequence (e.g., fully methylated sequence of 5 CpG sites). Thus, as each of the CpG sites of the K5n5 sequence is methylated, the resulting sequence corresponding to each CpG site may be “CG”, or a complement thereof. Thus, the probe may be designed to have a sequence that is complementary to each of the five “CG”, or complement thereof.

In various embodiments, an enrichment probe may be designed to be complementary to a target sequence derived from any of a K1n1 sequence, a K2n2 sequence, a K3n3 sequence, a K4n4 sequence, a K5n5 sequence, a K6n6 sequence, a K7n7 sequence, a K8n8 sequence, a K9n9 sequence, or a K10n10 sequence. In various embodiments, an enrichment probe may be designed to be complementary to a target sequence derived from any of a K2n1 sequence, a K3n1 sequence, a K4n1 sequence, a K5n1 sequence, a K6n1 sequence, a K7n1 sequence, a K8n1 sequence, a K9n1 sequence, a K10n1 sequence, a K3n2 sequence, a K4n2 sequence, a K5n2 sequence, a K6n2 sequence, a K7n2 sequence, a K8n2 sequence, a K9n2 sequence, a K10n2 sequence, a K4n3 sequence, a K5n3 sequence, a K6n3 sequence, a K7n3 sequence, a K8n3 sequence, a K9n3 sequence, a K10n3 sequence, a K5n4 sequence, a K6n4 sequence, a K7n4 sequence, a K8n4 sequence, a K9n4 sequence, a K10n4 sequence, a K6n5 sequence, a K7n5 sequence, a K8n5 sequence, a K9n5 sequence, a K10n5 sequence, a K7n6 sequence, a K8n6 sequence, a K9n6 sequence, a K10n6 sequence, a K8n7 sequence, a K9n7 sequence, a K10n7 sequence, a K9n8 sequence, a K10n8 sequence, or a K10n9 sequence.

In various embodiments, an enrichment probe includes a sequence that is between about 50 nucleotide bases and about 150 nucleotide bases. In various embodiments, an enrichment probe includes a sequence that is between about 55 nucleotide bases and about 120 nucleotide bases, between about 60 nucleotide bases and about 80 nucleotide bases, or between about 65 nucleotide bases and about 75 nucleotide bases. In various embodiments, an enrichment probe includes a sequence that is about 70 nucleotide bases. In various embodiments, an enrichment probe includes a sequence that is between about 80 nucleotide bases and about 120 nucleotide bases, between about 90 nucleotide bases and about 110 nucleotide bases, or between about 95 nucleotide bases and about 105 nucleotide bases. In various embodiments, an enrichment probe includes a sequence that is about 100 nucleotide bases.

In various embodiments, an enrichment probe further includes an affinity moiety. An affinity moiety can be biotin, streptavidin, heparin, an aptamer, a click-chemistry moiety, digoxigenin, primary amine(s), carboxyl(s), hydroxyl(s), aldehyde(s), ketone(s), or any combination thereof. In various embodiments, the enrichment probe includes an affinity moiety located at the 3′ end of the enrichment probe. In various embodiments, the enrichment probe includes an affinity moiety located at the 5′ end of the enrichment probe. In various embodiments, the enrichment probe includes an affinity moiety located on a nucleotide of the enrichment probe. In particular embodiments, the enrichment probe includes a biotin group. Thus, the enrichment probe can be captured by a corresponding affinity moiety (e.g., a streptavidin group) for purposes of performing enrichment. For example, a streptavidin coated solid support can be used to capture the enrichment probe and therefore, capture the hybridized target sequence for purposes of enrichment.

Generally, the enrichment probe is longer than a blocker sequence. In various embodiments, the enrichment probe is longer than every blocker sequence provided (e.g., blocker sequences provided as shown in FIG. 4A). In various embodiments, the enrichment probe is between about 2 times and about 8 times longer in length in comparison to each of the plurality of blocker sequences. In various embodiments, the enrichment probe is between about 3 times and about 7 times longer in length in comparison to each of the plurality of blocker sequences. In various embodiments, the enrichment probe is between about 4 times and about 6 times longer in length in comparison to each of the plurality of blocker sequences. In various embodiments, the enrichment probe is between about 2 times and about 8 times longer in length in comparison to an average length of the plurality of blocker sequences. In various embodiments, the enrichment probe is between about 3 times and about 7 times longer in length in comparison to an average length of the plurality of blocker sequences. In various embodiments, the enrichment probe is between about 4 times and about 6 times longer in length in comparison to an average length of the plurality of blocker sequences.

d. Enrichment Steps

In certain embodiments, the target sequence or a subset of target sequences in the nucleic acid can be enriched using an enrichment step. The enrichments step can be performed using any enrichment method known in the art. Non-limiting examples include hybrid capture, use of DNA-binding proteins to enrich a target sequence or a subset of target sequences, and nucleic acid amplification (e.g., polymerase chain reaction). In various embodiments, the additional enrichment step involves performing indexing PCR amplification e.g., using library adapters (e.g., P5/P7 adapters). Thus, selective amplification nucleic acids with hybridized primer sequences can be performed using the library adapters. In contrast, nucleic acids in which primer sequences are incapable of hybridizing with do not undergo PCR amplification.

One or more additional enrichment steps can be performed before or after the blocking of the unmethylated nucleic acids, as described herein. For example, in certain embodiments, the method comprises a first step of depleting a first subset of nucleic acids (e.g., unmethylated nucleic acids or converted nucleic acids derived from unmethylated nucleic acids), thereby leaving a second subset of nucleic acids (e.g., methylated nucleic acids or converted nucleic acids derived from methylated nucleic acids). The method comprises a second step of subjecting nucleic acid sequences comprising the target sequence to one or more additional enrichment steps to enrich for at least a subset of the target sequences.

In certain embodiments, the method comprises a first step of subjecting a plurality of nucleic acid molecules that include target sequences to an enrichment step to enrich for the target sequences. The method further comprises a second step of subjecting the plurality of nucleic acid molecules to the depletion method disclosed herein, which depletes a first subset of nucleic acids (e.g., unmethylated nucleic acids or converted nucleic acids derived from unmethylated nucleic acids) thereby leaving a second subset of nucleic acids (e.g., methylated nucleic acids or converted nucleic acids derived from methylated nucleic acids).

In certain embodiments, a target sequence or a subset of target sequences in the nucleic acid can be enriched by subjecting the nucleic acid comprising the target sequence or the subset of target sequences to hybrid capture. In hybrid capture, labeled (e.g., biotinylated) capture probes that can bind to one or more target sequences or subsets of target sequences are exposed to the nucleic acid comprising the one or more target sequences. The capture probes are specific to a sequence of interest, for example, a methylation pattern of interest that can be detected as a bisulfite-converted epitype. Examples of such hybrid capture probe sets include the KAPA HyperPrep KAPA HyperCap Workflow with HyperChoice Probes, Twist Bioscience Twist Fast Hybridization Custom Target Enrichment Panel, Integrated DNA technologies xGen Custom Hybridization Capture Panel, and SeqCAP Epi Enrichment System from Roche Diagnostics (Pleasanton, CA).

In various embodiments, the enrichment step includes capturing the target sequence or a subset of target sequences and performing one or more washes to remove nucleic acids that were not successfully captured. In various embodiments, the additional enrichment step includes capturing the target sequence or a subset of target sequences using an affinity moiety. An affinity moiety can be biotin, streptavidin, heparin, an aptamer, a click-chemistry moiety, digoxigenin, primary amine(s), carboxyl(s), hydroxyl(s), aldehyde(s), ketone(s), or any combination thereof. An one example, an enrichment probe, such as an enrichment probe 430 disclosed in FIG. 4B, can be biotinylated such that it can be captured onto a corresponding solid support or substrate coated with a corresponding affinity moiety (e.g., streptavidin).

In various embodiments, a solid support or substrate can be any physically separable solid to which an affinity moiety can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidene difluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, the solid phase can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). The affinity moiety may be linked to the solid support by covalent bonds or by non-covalent interactions and may be linked to the solid support directly or indirectly (e.g., via an intermediary agent such as a spacer molecule).

In particular embodiments, the solid support is a bead, such as a streptavidin-coated bead. In such embodiments, the probe, such as an enrichment probe 430 disclosed in FIG. 4B, is biotinylated such that it can be captured onto the bead via the corresponding streptavidin affinity moiety. Thus, in scenarios where the probe is hybridized with a target sequence or a subset of target sequences, the probe and hybridized target sequence can be captured by the bead via the corresponding streptavidin affinity moiety. Through one or more subsequent washes, nucleic acids that are not bound to the bead are removed, thereby enriching for the target sequence.

### Example Nucleic Acids and Methods for Converting Nucleic Acids

As discussed herein, step 120 in FIG. 1 involves converting nucleic acid molecules from the obtained sample. In various embodiments, converting nucleic acid molecules includes treating the nucleic acid molecules to capture methylation modifications. In various embodiments, converting nucleic acid molecules involves converting one or more unmethylated nucleotides (e.g., cytosines) to another nucleotide (a “converted nucleotide”, as used herein), e.g., using chemical or enzymatic means. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5mC)) is converted to a thymine, which pairs with adenine. In certain embodiments, methylated cytosines are protected from conversion (e.g., deamination) during the conversion step.

After a nucleic acid has been treated to convert unmethylated, or, in some cases, methylated nucleotides, into another nucleotide, the nucleic acid may be amplified. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

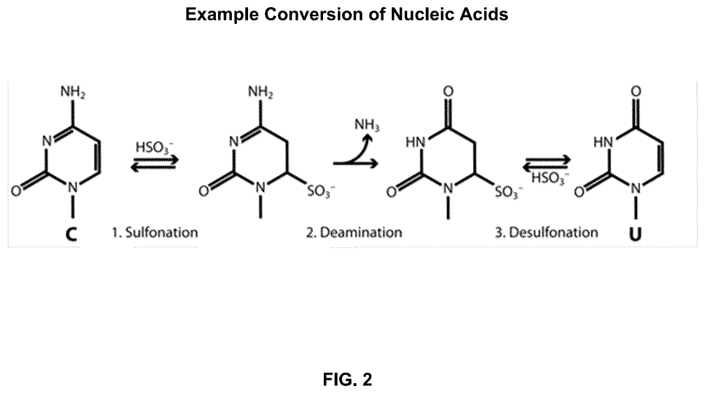

In certain aspects, conversion of the nucleic acids involves selectively deaminating nucleotides. FIG. 2 depicts an example conversion of nucleic acids, in accordance with an embodiment. Selective deamination refers to a process in which unmethylated cytosine residues are selectively deaminated over methylated cytosine (5-methylcytosine) residues. In certain embodiments, deamination of cytosine forms uracil, effectively inducing a C to T point mutation to allow for detection of methylated cytosines. Methods of deaminating cytosine are known in the art, and include chemical conversion (e.g., bisulfite conversion) and enzymatic conversion. In certain embodiments, the enzymatic conversion comprises subjecting the nucleic acid to TET2, which oxidizes methylated cytosines, thereby protecting them, and subsequent exposure to APOBEC, which converts unprotected (i.e., unmethylated) cytosines to uracils.

In some embodiments, the conversion, for example, bisulfite conversion or enzymatic conversion, uses commercially available kits. Bisulfite conversion can be performed using commercially available technologies, such as EZ DNA Methylation-Gold, EZ DNAMethylation-Direct or an EZ DNAMethylation-Lighting kit (Zymo Research Corp (Irvine, California)) or EpiTect Fast available from Qiagen (Germantown, MD). In another example a kit such as APOBECSeq (NEBiolabs) or OneStep qMethyl-PCR Kit (Zymo Research Corp (Irvine, California)) is used.

a. Source of Nucleic Acids

Nucleic acids used in the methods described herein can be derived from any source, such as a sample taken from the environment or from a subject (e.g., a human subject). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can take any of a variety of forms, such as a liquid biopsy (e.g., blood, urine, stool, saliva, or mucous), or a tissue biopsy, or other solid biopsy. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc.

The nucleic acid can be of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), and/or ribonucleic acid (RNA) and/or RNA analogs, all of which can be in single- or double-stranded form. In certain embodiments, single-stranded nucleic acids can be made double stranded prior to cutting with an enzyme. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids can comprise protein (e.g., histones, DNA binding proteins, and the like). Nucleic acids analyzed by processes described herein can be substantially isolated and are not substantially associated with protein or other molecules. Nucleic acids can also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides can include deoxyadenosine, deoxycytidine, deoxyguanosine and deoxythymidine. A nucleic acid may be prepared using a nucleic acid obtained from a subject as a template.

In certain embodiments, the nucleic acid is a cell-free nucleic acid, which can be found in bodily fluids such as blood, whole blood, plasma, serum, urine, cerebrospinal fluid, fecal, saliva, sweat, sweat, tears, pleural fluid, pericardial fluid, or peritoneal fluid of a subject. In certain embodiments, a plasma sample can be used directly in the methods disclosed herein (for example, in the cutting step), without prior purification or isolation of nucleic acids in the plasma. Cell-free nucleic acids originate from one or more healthy cells and/or from one or more cancer cells, or from non-human sources such bacteria, fungi, viruses. Examples of the cell-free nucleic acids include but are not limited to cell-free DNA (“cfDNA”), including mitochondrial DNA or genomic DNA, and cell-free RNA. In certain embodiments herein, instruments for assessing the quality of the cell-free nucleic acids, such as the TapeStation System from Agilent Technologies (Santa Clara, CA) can be used. Concentrating low-abundance cfDNA can be accomplished, for example using a Qubit Fluorometer from Thermofisher Scientific (Waltham, MA).

In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free).

A methylated nucleic acid is a nucleic acid having a modification in which a hydrogen atom on the pyrimidine ring of a cytosine base is converted to a methyl group, forming 5-methylcytosine. Methylation can occur at dinucleotides of cytosine and guanine referred to herein as “CpG sites”, which can be a target for enrichment. Methylation of cytosine can occur in cytosines in other sequence contexts, for example, 5′-CHG-3′ and 5′-CHH-3′, where His adenine, cytosine or thymine. Cytosine methylation can also be in the form of 5-hydroxymethylcytosine. Methylation of DNA can include methylation of non-cytosine nucleotides, such as N6-methyladenine (6 mA). Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

In certain embodiments, the nucleic acid comprises a CpG site (i.e., cytosine and guanine separated by only one phosphate group). In certain embodiments, the nucleic acid comprises a CpG island (also referred to as a “CG islands” or “CGI”) or a portion thereof, which is the target for enrichment. Because certain CGIs and certain features of certain CGIs in tumor cells tend to be different from the same CGIs or features of the CGIs in healthy cells, detection of such CGIs can be informative of a health condition. In certain embodiments, the CGI is a “cancer informative CGIs”, which is defined and described in more detail below. In certain embodiments, the CpG is an “informative CpG”, e.g., a “cancer informative CGI”. Such CGIs may have methylation patterns in tumor cells that are different from the methylation patterns in healthy cells. Accordingly, detection of a cancer informative CGI can be informative regarding a subject's risk of developing cancer or can be indicative that the subject has cancer. Exemplary cancer informative CGIs, which can be target sequences as described herein, are identified in, e.g., Table 1 of U.S. Patent Publication 2020/0109456A1 and Tables 2 and 3 of WO2022/133315, each of which are hereby incorporated by reference in its entirety. Further exemplary cancer informative CGIs are shown in Tables 1 and 2 included herein.

In certain aspects, the nucleic acids of the invention have been treated to convert one or more unmethylated nucleotides (e.g., cytosines) to another nucleotide (a “converted nucleotide”, as used herein, such as a uracil), for example, prior to amplification. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5mC)) is converted to a thymine, which pairs with adenine. In certain embodiments, methylated cytosines are protected from conversion (e.g., deamination) during the conversion step.

After a nucleic acid has been treated to convert unmethylated, or, in some cases, methylated nucleotides, into another nucleotide, the nucleic acid may be amplified. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

b. Bisulfite Conversion

Bisulfite conversion is performed on DNA by denaturation using high heat, preferential deamination (at an acidic pH) of unmethylated cytosines, which are then converted to uracil by desulfonation (at an alkaline pH). Methylated cytosines remain unchanged on the single-stranded DNA (ssDNA) product.

In some embodiments the methods include treatment of the sample with bisulfite (e.g., sodium bisulfite, potassium bisulfite, ammonium bisulfite, magnesium bisulfite, sodium metabisulfite, potassium metabisulfite, ammonium metabisulfite, magnesium metabisulfite and the like). Unmethylated cytosine is converted to uracil through a three-step process during sodium bisulfite modification. As shown in FIG. 2, the steps are sulphonation to convert cytosine to cytosine sulphonate, deamination to convert cytosine sulphonate to uracil sulphonate and alkali desulphonation to convert uracil sulphonate to uracil. Conversion on methylated cytosine is much slower and is not observed at significant levels in a 4-16 hour reaction. (See Clark et al., Nucleic Acids Res., 22 (15): 2990-7 (1994).) If the cytosine is methylated it will remain a methylated cytosine. If the cytosine is unmethylated it will be converted to uracil. When the modified strand is copied, for example, through extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated and converted to U. When the double stranded extension product is amplified those Cs that were converted to Us and resulted in incorporation of A in the extended primer will be replaced by Ts during amplification. Those Cs that were not converted (i.e., the methylated Cs) and resulted in the incorporation of G will be replaced by unmethylated Cs during amplification.

c. Enzymatic Conversion

In certain embodiments, the enzymatic treatment with a cytidine deaminase enzyme is used to convert cytosine to uracil. Enzymatic conversion can include an oxidation step, in which Tet methylcytosine dioxygenase 2 (TET2) catalyzes the oxidation of 5mC to 5hmC to protect methylated cytosines from conversion by subsequent exposure to a cytidine deaminase. Other protection steps known in the art can be used in addition to or in place of oxidation by TET2. After the oxidation step, the nucleic acid is treated with the cytidine deaminase to convert one or more unmethylated cytosines to uracils. As with bisulfite conversion, when the modified strand is copied, a G will be incorporated in the interrogation position (opposite the C being interrogated) if the C was methylated and an A will be incorporated in the interrogation position if the C was unmethylated. When the double stranded extension product is amplified those Cs that were converted to Us and resulted in incorporation of A in the extended primer will be replaced by Ts during amplification. Those Cs that were not modified and resulted in the incorporation of G will remain as C.

In certain embodiments the cytidine deaminase may be APOBEC. In certain embodiments the cytidine deaminase includes activation induced cytidine deaminase (AID) and apolipoprotein B mRNA editing enzymes, catalytic polypeptide-like (APOBEC). In certain embodiments, the APOBEC enzyme is selected from the human APOBEC family consisting of: APOBEC-1 (Apo1), APOBEC-2 (Apo2), AID, APOBEC-3A, -3B, -3C, -3DE, -3F, -3G, -3H and APOBEC-4 (Apo4). In certain embodiments, the APOBEC enzyme is APOBEC-seq.

d. Nitrite Conversion

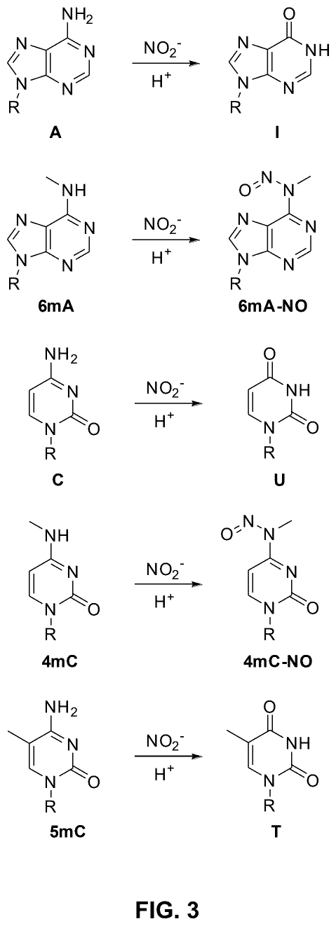

In certain embodiments, nitrite treatment is used to deaminate adenine and cytosine. As shown in FIG. 3, deamination of an A results in conversion to an inosine (I), which is read by a polymerase as a G, whereas deamination of a methylated A (N⁶-methyladenine (6 mA)) results in a nitrosylated 6 mA (6 mA-NO), which causes the base to be read by a polymerase as an A. Deamination of a C results in conversion to a uracil, which is read by a polymerase as a T, whereas deamination of a N⁴-methylcytosine (4mC) to 4mC-NO or a 5-methylcytosine (5mC) to a T causes the base to be read by a polymerase as a C or a T, respectively. For 5mC bases, the C to T ratio at the 5mC position is about 40% higher than other cytosine positions, allowing 5mC to be differentiated from C. (Sec, Li et al. (2022) Genome Biology 23:122.)

### Additional Embodiments

Disclosed herein is a method for enriching for a target nucleic acid sequence, the method comprising: obtaining a mixture of nucleic acid sequences, the mixture comprising the target nucleic acid sequence and one or more variant sequences of the target nucleic acid sequence; providing an enrichment probe comprising a sequence complementary to the target nucleic acid sequence; providing a plurality of blocker probes comprising a sequence complementary to one or more variant sequences of the target nucleic acid sequence; and selectively enriching for the target nucleic acid sequence using the enrichment probe without enriching for the one or more variant sequences of the target nucleic acid sequence.

In various embodiments, the target nucleic acid sequence comprises a sequence comprising one or more methylated CpG sites, or a sequence derived from the sequence comprising one or more methylated CpG sites. In various embodiments, the target nucleic acid sequence comprises a sequence comprising at least five consecutive methylated CpG sites, or a sequence derived from the sequence comprising at least five consecutive methylated CpG sites. In various embodiments, the one or more variant sequences of the target nucleic acid sequence comprise one or more unmethylated CpG sites. In various embodiments, the one or more variant sequences and the target nucleic acid sequence differ by one or more methylated or unmethylated CpG sites.

In various embodiments, the enrichment probe is at least 50 nucleotide bases, at least 70 nucleotide bases, or at least 100 nucleotide bases in length. In various embodiments, the plurality of blocker probes are between 10 and 20 nucleotide bases in length. In various embodiments, the plurality of blocker probes are between 15 and 18 nucleotide bases in length. In various embodiments, selectively enriching for the target nucleic acid sequence using the enrichment probe without enriching for the one or more variant sequences of the target nucleic acid sequence comprises: hybridizing the enrichment probe to the target nucleic acid sequence; hybridizing one or more blocker probes to the one or more variant sequences of the target nucleic acid sequence; and enriching for the hybridized enrichment probe while the one or more hybridized blocker probes prevents enrichment for the one or more variant sequences of the target nucleic acid sequence. In various embodiments, selectively enriching for the target nucleic acid sequence using the enrichment probe without enriching for the one or more variant sequences of the target nucleic acid sequence comprises: binding a streptavidin bead to a biotin group of the enrichment probe; and washing and removing the plurality of blocker probes and/or the one or more variant sequences of the target nucleic acid sequence.

### EXAMPLES

Practice of embodiments disclosed herein will be more fully understood from the foregoing examples, which are presented herein for illustrative purposes only, and should not be construed as limiting the invention in any way.

### Example 1—General Methods for Enriching for Target Nucleic Acid Molecules

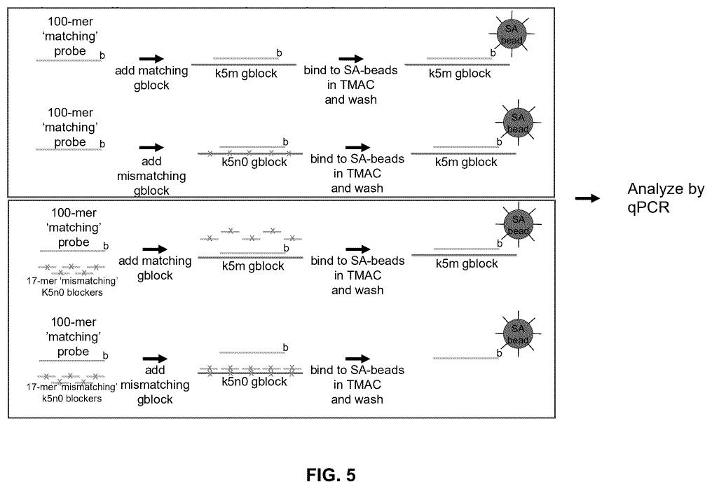

FIG. 5 depicts an overall schematic involving provision of probes (e.g., 70 base or 100 base probes) and/or 17-mer mismatching K5n0 blockers for enrichment of target sequences (e.g., target sequences corresponding to fully methylated sequences).

Purpose: Test binding of 70 base and 100 base probe to Km10C- and Km10T-gblocks in TMAC and binding to SA-beads at 52° C. in the presence of blockers. Table 1 below shows the experimental conditions that were tested.

Protocol: Use 5 million copies gblock as target. Use 100 million copies of probe as capture. Denaturing at 95° C. in TMAC or Tris buffers and slowly cool to 52° C. and incubate overnight. Then incubate with SA-beads at room temperature for 30 minutes while rotating. Collect supernatants on magnet. Wash beads. Ethanol ppt the supernatants and resuspend. Measure supernatants and beads for gblock using qPCR Km10 assays.

### Example Protocol

1. Prepare Buffers: The TMAC buffer comes pre-made and is 3 M TMAC with, 1 mM EDTA, 0.1% SDS.
   a. Tris Incubation Buffer: 50 mM Tris, pH 8.0, 1 mM EDTA, 0.1% SDS.
   b. Tris Wash Buffer: 50 mM Tris, pH 8.0, 1 mM EDTA.
2. Use the Km10C- and Km10T-gblocks at 1 million copies/uL.
3. Dilute the probe to 100 million copies/uL. The probe is starting at 6.03E13 copies/uL.
4. Diluge blocker to 100 million copies/uL.
5. Mix:
   a. 5 uL Target DNA
   b. 1 uL probe
   c. 1 uL blockers or water
   d. 40 uL buffer
6. Incubate 95° C. for 5 minutes on heat block. Cool to 52° C. over the course of 15 minutes.
7. Incubate overnight.
8. Prepare SA-beads: Use MyOne SA beads. Wash beads in either TMAC Buffer or Tris Incubation Buffer 3×. Prepare 10 uL beads per reaction.
9. Take off heat and add magnetic beads. Mix.
10. Place on rotator and mix for 30 minutes.
11. Place on Magnetic stand and remove supernatant. Save supernatant for ethanol ppt.
12. Add wash buffer and return rotator for 5 minutes.
13. Place on Magnetic stand and remove supernatant.
14. Resuspend in wash buffer.
15. For all supernatant samples, EtOH ppt. and resuspend in 50 uL TE.
16. Analyze 1 uL using Km10 qPCR probe sets.

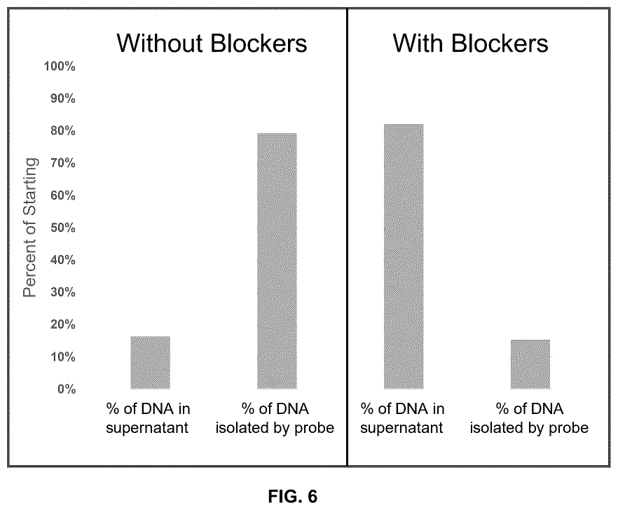

Reference is now made to FIG. 6, which shows the percentage of DNA (from a mismatching g-block) isolated by an enrichment probe with and without blockers. Here, 17-mer blockers were either provided or withheld. Provided 17-mer blockers prevent mismatching sequences from binding to long-probes. Long probes were either 70-mer or 100-mer biotinylated probes with matching or mismatching gblocks. Samples were incubated at 52° C. for 16 hours in TMAC to enable hybridization. Next, probes were bound to SA beads at room temperature, followed by a wash, ethanol precipitation. qPCR was performed to measure the % of DNA in supernatant and beads.

As shown in FIG. 6, the inclusion of blocker sequences reduced the quantity of isolated DNA (from mismatching g-blocks) containing mismatches. The inclusion of blocker sequences did not prevent the probe from isolating DNA that does not have mismatches.

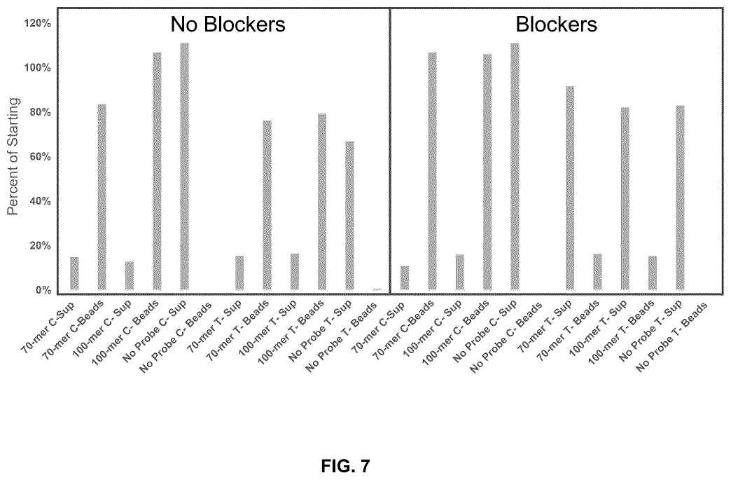

Reference is now made to FIG. 7, which shows the percentage of DNA isolated by various probes (e.g., 70-mer and 100-mer probes) with and without blockers. Here, 17-mer blockers were either provided or withheld. Provided 17-mer blockers prevent mismatching sequences from binding to long-probes. Long probes were either 70-mer or 100-mer biotinylated probes with matching or mismatching gblocks. Samples were incubated at 52° C. for 16 hours in TMAC to enable hybridization. Next, probes were bound to SA beads at room temperature, followed by a wash, ethanol precipitation. qPCR was performed to measure the % of DNA in supernatant (referred to as “Sup” in FIG. 7) and beads. Mismatch g-blocks are referred in FIG. 6 with a “T” (e.g., “70-mer T-Sup”) and non-mismatch g-blocks are referred in FIG. 6 with a “C” (e.g., “70-mer C-Sup”).

As shown in FIG. 7, the inclusion of blocker sequences reduced the quantity of isolated DNA (from mismatching g-blocks) containing mismatches. The inclusion of blocker sequences did not prevent the probe from isolating DNA that does not have mismatches. For example, see the control conditions of DNA without mismatch in the presence of blockers (labeled as “70-mer C-Beads” and “100-mer C-Beads”).

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### Tables

TABLE 1
Example experimental conditions

| | | | | Pre | |
|---|---|---|---|---|---|
| | | | | treatment | Incubation |
| Sample | gblock | Probe Set | Blockers? | gblock | Buffer |
| 1 | 5 × 10⁶ | 1 × 10⁸ | No | 95° C. | TMAC |
| | copies | copies 70 | | | |
| | Km10C- | base Km10C- | | | |
| 2 | 5 × 10⁶ | 1 × 10⁸ | No | 95° C. | TMAC |
| | copies | copies 70 | | | |
| | Km10T- | base Km10C- | | | |
| 3 | 5 × 10⁶ | 1 × 10⁸ | No | 95° C. | TMAC |
| | copies | copies 100 | | | |
| | Km10C- | base Km10C- | | | |
| 4 | 5 × 10⁶ | 1 × 10⁸ | No | 95° C. | TMAC |
| | copies | copies 100 | | | |
| | Km10T- | base Km10C- | | | |
| 5 | 5 × 10⁶ | — | No | 95° C. | TMAC |
| | copies | | | | |
| | Km10C- | | | | |
| 6 | 5 × 10⁶ | — | No | 95° C. | TMAC |
| | copies | | | | |
| | Km10T- | | | | |
| 7 | 5 × 10⁶ | 1 × 10⁸ | Yes | 95° C. | TMAC |
| | copies | copies 70 | | | |
| | Km10C- | base Km10C- | | | |
| 8 | 5 × 10⁶ | 1 × 10⁸ | Yes | 95° C. | TMAC |
| | copies | copies 70 | | | |
| | Km10T- | base Km10C- | | | |
| 9 | 5 × 10⁶ | 1 × 10⁸ | Yes | 95° C. | TMAC |
| | copies | copies 100 | | | |
| | Km10C- | base Km10C- | | | |
| 10 | 5 × 10⁶ | 1 × 10⁸ | Yes | 95° C. | TMAC |
| | copies | copies 100 | | | |
| | Km10T- | base Km10C- | | | |
| 11 | 5 × 10⁶ | — | Yes | 95° C. | TMAC |
| | copies | | | | |
| | Km10C- | | | | |
| 12 | 5 × 10⁶ | — | Yes | 95° C. | TMAC |
| | copies | | | | |
| | Km10T- | | | | |
| 13 | 5 × 10⁶ | 1 × 10⁸ | No | 95° C. | Tris |
| | copies | copies 17 | | | |
| | Km10C- | base 5-plex | | | |
| 14 | 5 × 10⁶ | 1 × 10⁸ | No | 95° C. | Tris |
| | copies | copies 17 | | | |
| | Km10T- | base 5-plex | | | |
| 15 | 5 × 10⁶ | 1 × 10⁸ | No | 95° C. | TMAC |
| | copies | copies 17 | | | |
| | Km10C- | base 5-plex | | | |
| 16 | 5 × 10⁶ | 1 × 10⁸ | No | 95° C. | TMAC |
| | copies | copies 17 | | | |
| | Km10T- | base 5-plex | | | |

| | Incubation | SA- | | | |
|---|---|---|---|---|---|
| Sample | Temperature | beads | Wash | EtOH ppt. | qPCR |
| 1 | 52° C. | Yes | Tris | Supernatant | SA-Beads and |
| | | | | | Supernatant |
| 2 | 52° C. | Yes | Tris | Supernatant | SA-Beads and |
| | | | | | Supernatant |
| 3 | 52° C. | Yes | Tris | Supernatant | SA-Beads and |
| | | | | | Supernatant |
| 4 | 52° C. | Yes | Tris | Supernatant | SA-Beads and |
| | | | | | Supernatant |
| 5 | 52° C. | Yes | Tris | Supernatant | SA-Beads and |
| | | | | | Supernatant |
| 6 | 52° C. | Yes | Tris | Supernatant | SA-Beads and |
| | | | | | Supernatant |
| 7 | 52° C. | Yes | Tris | Supernatant | SA-Beads and |
| | | | | | Supernatant |
| 8 | 52° C. | Yes | Tris | Supernatant | SA-Beads and |
| | | | | | Supernatant |
| 9 | 52° C. | Yes | Tris | Supernatant | SA-Beads and |
| | | | | | Supernatant |
| 10 | 52° C. | Yes | Tris | Supernatant | SA-Beads and |
| | | | | | Supernatant |
| 11 | 52° C. | Yes | Tris | Supernatant | SA-Beads and |
| | | | | | Supernatant |
| 12 | 52° C. | Yes | Tris | Supernatant | SA-Beads and |
| | | | | | Supernatant |
| 13 | 52° C. | Yes | Tris | Supernatant | SA-Beads and |
| | | | | | Supernatant |
| 14 | 52° C. | Yes | Tris | Supernatant | SA-Beads and |
| | | | | | Supernatant |
| 15 | 52° C. | Yes | Tris | Supernatant | SA-Beads and |
| | | | | | Supernatant |
| 16 | 52° C. | Yes | Tris | Supernatant | SA-Beads and |
| | | | | | Supernatant |

Note:
Samples 13-16 are a side experiment to test short probes in TMAC. No blockers are used.

TABLE 2
Genomic ranges of low background regions including a plurality
of CpG sites mapped to human genome, hg19. The “Genomic
Coordinate Start” and “Genomic Coordinate End”
columns indicate the beginning and end, respectively of
a range of genomic locations within a chromosome (“Chrom.”).

| | Genomic | Genomic |
|---|---|---|
| | Coordinate | Coordinate |
| Chromosome | Start | End |
| chr1 | 119527182 | 119527632 |
| chr1 | 119530152 | 119530432 |
| chr1 | 146551368 | 146551898 |
| chr1 | 1475454 | 1476214 |
| chr1 | 156863265 | 156863635 |
| chr1 | 203044592 | 203045022 |
| chr1 | 221067408 | 221067838 |
| chr1 | 243646244 | 243646754 |
| chr1 | 36042742 | 36043444 |
| chr1 | 41284067 | 41284677 |
| chr1 | 44883136 | 44884106 |
| chr1 | 46913816 | 46914343 |
| chr1 | 48058803 | 48059230 |
| chr1 | 63795374 | 63795714 |
| chr1 | 65990821 | 65991721 |
| chr1 | 67217899 | 67218409 |
| chr1 | 91183242 | 91183602 |
| chr1 | 91183712 | 91183962 |
| chr10 | 101290401 | 101291151 |
| chr10 | 102894100 | 102894360 |
| chr10 | 102894700 | 102895270 |
| chr10 | 103044090 | 103044310 |
| chr10 | 123922730 | 123923570 |
| chr10 | 129534690 | 129534920 |
| chr10 | 16562214 | 16562454 |
| chr11 | 31825753 | 31826243 |
| chr11 | 32354800 | 32355390 |
| chr11 | 32448451 | 32449041 |
| chr11 | 62693373 | 62693983 |
| chr12 | 114846931 | 114847561 |
| chr12 | 52652198 | 52652558 |
| chr12 | 57618699 | 57619019 |
| chr12 | 58021294 | 58021744 |
| chr12 | 6664425 | 6665336 |
| chr12 | 81102144 | 81102474 |
| chr13 | 109147608 | 109149208 |
| chr13 | 112712154 | 112712994 |
| chr13 | 112720604 | 112721334 |
| chr13 | 58203376 | 58204016 |
| chr13 | 58204026 | 58204296 |
| chr14 | 101193021 | 101193501 |
| chr14 | 24803748 | 24804358 |
| chr14 | 37126588 | 37127068 |
| chr14 | 38724554 | 38725594 |
| chr15 | 76630599 | 76630959 |
| chr15 | 76635109 | 76635319 |
| chr15 | 89922206 | 89922586 |
| chr16 | 31580559 | 31581023 |
| chr16 | 54970251 | 54970581 |
| chr16 | 54970951 | 54971581 |
| chr16 | 66612869 | 66613229 |
| chr16 | 67197082 | 67197882 |
| chr16 | 86612428 | 86612848 |
| chr17 | 32483927 | 32484257 |
| chr17 | 37320732 | 37321812 |
| chr17 | 46618927 | 46619367 |
| chr17 | 46711072 | 46711352 |
| chr17 | 5000129 | 5000939 |
| chr19 | 13210053 | 13210423 |
| chr19 | 41119211 | 41120121 |
| chr19 | 46916611 | 46916971 |
| chr19 | 46996437 | 46997247 |
| chr19 | 55598337 | 55598657 |
| chr2 | 105459037 | 105459557 |
| chr2 | 105459567 | 105459847 |
| chr2 | 106681872 | 106682322 |
| chr2 | 114257035 | 114257555 |
| chr2 | 128421729 | 128421959 |
| chr2 | 171679398 | 171679898 |
| chr2 | 171679928 | 171680218 |
| chr2 | 219735672 | 219736742 |
| chr2 | 45228144 | 45228934 |
| chr2 | 468079 | 468609 |
| chr2 | 63281024 | 63281294 |
| chr2 | 66808328 | 66809548 |
| chr2 | 73147405 | 73148085 |
| chr2 | 80530337 | 80530777 |
| chr3 | 127795639 | 127796109 |
| chr3 | 129693197 | 129693897 |
| chr3 | 129693987 | 129694547 |
| chr3 | 138657297 | 138657517 |
| chr3 | 138657887 | 138658147 |
| chr3 | 138658417 | 138658807 |
| chr3 | 194118681 | 194118988 |
| chr4 | 13523882 | 13524592 |
| chr4 | 140200464 | 140200994 |
| chr4 | 154713542 | 154714062 |
| chr4 | 155662869 | 155664039 |
| chr4 | 174430584 | 174431044 |
| chr4 | 174459004 | 174459374 |
| chr4 | 85402970 | 85403220 |
| chr5 | 115151898 | 115152713 |
| chr5 | 145725379 | 145725679 |
| chr5 | 172659649 | 172659909 |
| chr5 | 37834801 | 37835021 |
| chr5 | 42994626 | 42994936 |
| chr5 | 42995122 | 42995415 |
| chr5 | 45696174 | 45696664 |
| chr6 | 10381538 | 10381798 |
| chr6 | 137244265 | 137244585 |
| chr6 | 1383915 | 1384335 |
| chr6 | 1393915 | 1394325 |
| chr6 | 150285942 | 150286515 |
| chr6 | 1619923 | 1620423 |
| chr6 | 26240697 | 26240951 |
| chr6 | 26614073 | 26614293 |
| chr6 | 26614343 | 26614843 |
| chr6 | 38682939 | 38683149 |
| chr6 | 42071932 | 42072572 |
| chr6 | 6003827 | 6004627 |
| chr6 | 78172201 | 78172601 |
| chr7 | 12151220 | 12151550 |
| chr7 | 156797835 | 156798085 |
| chr7 | 156798185 | 156798495 |
| chr7 | 156814492 | 156814593 |
| chr7 | 24323558 | 24324008 |
| chr7 | 27203981 | 27205891 |
| chr7 | 27279145 | 27279415 |
| chr7 | 42267516 | 42267936 |
| chr7 | 64349424 | 64349914 |
| chr8 | 139509105 | 139509455 |
| chr8 | 23563975 | 23564325 |
| chr8 | 65282023 | 65282443 |
| chr8 | 70981763 | 70982053 |
| chr8 | 70984243 | 70984513 |
| chr8 | 72468930 | 72469130 |
| chr8 | 97157293 | 97158030 |
| chr8 | 97170421 | 97170981 |
| chr9 | 126774736 | 126775106 |
| chr9 | 129381027 | 129381477 |

TABLE 3
Exemplary Target Regions
Human universal genomic locations and CpG sites mapped
to human genome, hg38. The “Start” and “End”
columns indicate the beginning and end, respectively
of a range of a target region within a chromosome (“Chrom”).

| Chrom | Start | End | Assembly |
|---|---|---|---|
| chr1 | 1540074 | 1540834 | hg38 |
| chr1 | 3070966 | 3070973 | hg38 |
| chr1 | 3070994 | 3071005 | hg38 |
| chr1 | 3071013 | 3071035 | hg38 |
| chr1 | 18629812 | 18629928 | hg38 |
| chr1 | 18630222 | 18630341 | hg38 |
| chr1 | 31772758 | 31772784 | hg38 |
| chr1 | 31772823 | 31772852 | hg38 |
| chr1 | 35576832 | 35576911 | hg38 |
| chr1 | 35577049 | 35577086 | hg38 |
| chr1 | 35577126 | 35577843 | hg38 |
| chr1 | 37764229 | 37764238 | hg38 |
| chr1 | 40738897 | 40738898 | hg38 |
| chr1 | 40818395 | 40819005 | hg38 |
| chr1 | 44417464 | 44418434 | hg38 |
| chr1 | 46448144 | 46448671 | hg38 |
| chr1 | 46485917 | 46486000 | hg38 |
| chr1 | 47229217 | 47229248 | hg38 |
| chr1 | 47443734 | 47443758 | hg38 |
| chr1 | 47443919 | 47443926 | hg38 |
| chr1 | 47444080 | 47444088 | hg38 |
| chr1 | 47446268 | 47446321 | hg38 |
| chr1 | 47593131 | 47593558 | hg38 |
| chr1 | 50415335 | 50415436 | hg38 |
| chr1 | 50415694 | 50415708 | hg38 |
| chr1 | 50416018 | 50416149 | hg38 |
| chr1 | 50417154 | 50417327 | hg38 |
| chr1 | 50417357 | 50417465 | hg38 |
| chr1 | 50420990 | 50421009 | hg38 |
| chr1 | 50421155 | 50421160 | hg38 |
| chr1 | 50421207 | 50421249 | hg38 |
| chr1 | 50421431 | 50421445 | hg38 |
| chr1 | 50421483 | 50421499 | hg38 |
| chr1 | 50425624 | 50425709 | hg38 |
| chr1 | 63318755 | 63318811 | hg38 |
| chr1 | 63319517 | 63319549 | hg38 |
| chr1 | 63319584 | 63319624 | hg38 |
| chr1 | 63319650 | 63319669 | hg38 |
| chr1 | 63320266 | 63320276 | hg38 |
| chr1 | 63320303 | 63320346 | hg38 |
| chr1 | 63320369 | 63320408 | hg38 |
| chr1 | 63320630 | 63320645 | hg38 |
| chr1 | 63321231 | 63321273 | hg38 |
| chr1 | 63321378 | 63321424 | hg38 |
| chr1 | 63321602 | 63321618 | hg38 |
| chr1 | 63321647 | 63321731 | hg38 |
| chr1 | 63326165 | 63326219 | hg38 |
| chr1 | 63327127 | 63327200 | hg38 |
| chr1 | 63329703 | 63330083 | hg38 |
| chr1 | 63330357 | 63330398 | hg38 |
| chr1 | 63330403 | 63330419 | hg38 |
| chr1 | 65525138 | 65526038 | hg38 |
| chr1 | 66752108 | 66752149 | hg38 |
| chr1 | 66752216 | 66752726 | hg38 |
| chr1 | 75130252 | 75130263 | hg38 |
| chr1 | 87151474 | 87151518 | hg38 |
| chr1 | 90706663 | 90706703 | hg38 |
| chr1 | 90717473 | 90717535 | hg38 |
| chr1 | 90717685 | 90718045 | hg38 |
| chr1 | 90718086 | 90718104 | hg38 |
| chr1 | 90718155 | 90718405 | hg38 |
| chr1 | 90719213 | 90719227 | hg38 |
| chr1 | 90719951 | 90719995 | hg38 |
| chr1 | 90725003 | 90725026 | hg38 |
| chr1 | 90725027 | 90725050 | hg38 |
| chr1 | 90725063 | 90725091 | hg38 |
| chr1 | 90726211 | 90726241 | hg38 |
| chr1 | 90726994 | 90727036 | hg38 |
| chr1 | 110068031 | 110068065 | hg38 |
| chr1 | 110068092 | 110068131 | hg38 |
| chr1 | 110068174 | 110068208 | hg38 |
| chr1 | 110068218 | 110068252 | hg38 |
| chr1 | 110068375 | 110068444 | hg38 |
| chr1 | 110068507 | 110068597 | hg38 |
| chr1 | 110068729 | 110068752 | hg38 |
| chr1 | 110068760 | 110068898 | hg38 |
| chr1 | 110068906 | 110068929 | hg38 |
| chr1 | 110068930 | 110068949 | hg38 |
| chr1 | 110069310 | 110069327 | hg38 |
| chr1 | 110069520 | 110069554 | hg38 |
| chr1 | 110070205 | 110070278 | hg38 |
| chr1 | 110151221 | 110151246 | hg38 |
| chr1 | 111270940 | 111270974 | hg38 |
| chr1 | 113973095 | 113973096 | hg38 |
| chr1 | 118979549 | 118979568 | hg38 |
| chr1 | 118979821 | 118979847 | hg38 |
| chr1 | 118979879 | 118979941 | hg38 |
| chr1 | 118979954 | 118979972 | hg38 |
| chr1 | 118979984 | 118980007 | hg38 |
| chr1 | 118980105 | 118980114 | hg38 |
| chr1 | 118983368 | 118983436 | hg38 |
| chr1 | 118983450 | 118983513 | hg38 |
| chr1 | 118983563 | 118983633 | hg38 |
| chr1 | 118983814 | 118983857 | hg38 |
| chr1 | 118984178 | 118984199 | hg38 |
| chr1 | 118984469 | 118984482 | hg38 |
| chr1 | 118984559 | 118985009 | hg38 |
| chr1 | 118985187 | 118985204 | hg38 |
| chr1 | 118985395 | 118985476 | hg38 |
| chr1 | 118986004 | 118986057 | hg38 |
| chr1 | 118986406 | 118986458 | hg38 |
| chr1 | 118986737 | 118986774 | hg38 |
| chr1 | 118987529 | 118987809 | hg38 |
| chr1 | 118989052 | 118989082 | hg38 |
| chr1 | 118989375 | 118989386 | hg38 |
| chr1 | 118989493 | 118989525 | hg38 |
| chr1 | 118990346 | 118990360 | hg38 |
| chr1 | 118992607 | 118992639 | hg38 |
| chr1 | 118992654 | 118992697 | hg38 |
| chr1 | 118993044 | 118993094 | hg38 |
| chr1 | 118993117 | 118993137 | hg38 |
| chr1 | 118993253 | 118993269 | hg38 |
| chr1 | 118993298 | 118993340 | hg38 |
| chr1 | 119000685 | 119000714 | hg38 |
| chr1 | 119007847 | 119007883 | hg38 |
| chr1 | 119008088 | 119008100 | hg38 |
| chr1 | 147079643 | 147079671 | hg38 |
| chr1 | 147079812 | 147080349 | hg38 |
| chr1 | 147080795 | 147080842 | hg38 |
| chr1 | 150226791 | 150226792 | hg38 |
| chr1 | 151838727 | 151838748 | hg38 |
| chr1 | 155295219 | 155295236 | hg38 |
| chr1 | 155322373 | 155322418 | hg38 |
| chr1 | 156388010 | 156388062 | hg38 |
| chr1 | 156420177 | 156420266 | hg38 |
| chr1 | 156845124 | 156845158 | hg38 |
| chr1 | 156845162 | 156845188 | hg38 |
| chr1 | 156845601 | 156845634 | hg38 |
| chr1 | 156845681 | 156845738 | hg38 |
| chr1 | 156845788 | 156845851 | hg38 |
| chr1 | 156845892 | 156845922 | hg38 |
| chr1 | 156845939 | 156845955 | hg38 |
| chr1 | 156893337 | 156893462 | hg38 |
| chr1 | 156893473 | 156893843 | hg38 |
| chr1 | 160333626 | 160333627 | hg38 |
| chr1 | 161958537 | 161958538 | hg38 |
| chr1 | 165354259 | 165354299 | hg38 |
| chr1 | 165354395 | 165354432 | hg38 |
| chr1 | 165354438 | 165354462 | hg38 |
| chr1 | 165354957 | 165354999 | hg38 |
| chr1 | 165355396 | 165355469 | hg38 |
| chr1 | 165355514 | 165355521 | hg38 |
| chr1 | 170660331 | 170660377 | hg38 |
| chr1 | 170660450 | 170660475 | hg38 |
| chr1 | 170660903 | 170660940 | hg38 |
| chr1 | 170661002 | 170661049 | hg38 |
| chr1 | 170661413 | 170661438 | hg38 |
| chr1 | 170661600 | 170661606 | hg38 |
| chr1 | 171786030 | 171786031 | hg38 |
| chr1 | 175599285 | 175599316 | hg38 |
| chr1 | 175599426 | 175599489 | hg38 |
| chr1 | 175599495 | 175599530 | hg38 |
| chr1 | 180233305 | 180233342 | hg38 |
| chr1 | 180233717 | 180233729 | hg38 |
| chr1 | 181482182 | 181482265 | hg38 |
| chr1 | 181482267 | 181482342 | hg38 |
| chr1 | 181482371 | 181482399 | hg38 |
| chr1 | 181483596 | 181483624 | hg38 |
| chr1 | 183417139 | 183417192 | hg38 |
| chr1 | 183417456 | 183417471 | hg38 |
| chr1 | 184708330 | 184708331 | hg38 |
| chr1 | 197040668 | 197040669 | hg38 |
| chr1 | 197910570 | 197910588 | hg38 |
| chr1 | 197910590 | 197910612 | hg38 |
| chr1 | 197919660 | 197919761 | hg38 |
| chr1 | 197919764 | 197919797 | hg38 |
| chr1 | 197919934 | 197919954 | hg38 |
| chr1 | 197919991 | 197920014 | hg38 |
| chr1 | 197920479 | 197920540 | hg38 |
| chr1 | 200040860 | 200040885 | hg38 |
| chr1 | 200042911 | 200042922 | hg38 |
| chr1 | 203075464 | 203075894 | hg38 |
| chr1 | 205343478 | 205343497 | hg38 |
| chr1 | 205343797 | 205343809 | hg38 |
| chr1 | 207910685 | 207910727 | hg38 |
| chr1 | 213980132 | 213980180 | hg38 |
| chr1 | 213985453 | 213985483 | hg38 |
| chr1 | 217134081 | 217134102 | hg38 |
| chr1 | 217134160 | 217134179 | hg38 |
| chr1 | 217136473 | 217136526 | hg38 |
| chr1 | 217136931 | 217136957 | hg38 |
| chr1 | 217137436 | 217137474 | hg38 |
| chr1 | 217137627 | 217137651 | hg38 |
| chr1 | 217137762 | 217137800 | hg38 |
| chr1 | 217138126 | 217138190 | hg38 |
| chr1 | 217138328 | 217138343 | hg38 |
| chr1 | 217138357 | 217138376 | hg38 |
| chr1 | 217139702 | 217139717 | hg38 |
| chr1 | 217139724 | 217139736 | hg38 |
| chr1 | 217140200 | 217140261 | hg38 |
| chr1 | 217140565 | 217140626 | hg38 |
| chr1 | 220877149 | 220877181 | hg38 |
| chr1 | 220877230 | 220877266 | hg38 |
| chr1 | 220877289 | 220877303 | hg38 |
| chr1 | 220891644 | 220891671 | hg38 |
| chr1 | 220891888 | 220891981 | hg38 |
| chr1 | 220894066 | 220894496 | hg38 |
| chr1 | 224617083 | 224617105 | hg38 |
| chr1 | 224617415 | 224617428 | hg38 |
| chr1 | 224618133 | 224618148 | hg38 |
| chr1 | 225677371 | 225677454 | hg38 |
| chr1 | 225677546 | 225677580 | hg38 |
| chr1 | 228464803 | 228464814 | hg38 |
| chr1 | 228650240 | 228650254 | hg38 |
| chr1 | 228650261 | 228650301 | hg38 |
| chr1 | 229431623 | 229431632 | hg38 |
| chr1 | 229431694 | 229431806 | hg38 |
| chr1 | 229432112 | 229432127 | hg38 |
| chr1 | 229432165 | 229432172 | hg38 |
| chr1 | 229432263 | 229432280 | hg38 |
| chr1 | 229432604 | 229432620 | hg38 |
| chr1 | 229602423 | 229602424 | hg38 |
| chr1 | 231160894 | 231160917 | hg38 |
| chr1 | 232515004 | 232515005 | hg38 |
| chr1 | 233905514 | 233905525 | hg38 |
| chr1 | 233905647 | 233905671 | hg38 |
| chr1 | 233905694 | 233905711 | hg38 |
| chr1 | 234393824 | 234393825 | hg38 |
| chr1 | 235650997 | 235651027 | hg38 |
| chr1 | 237041827 | 237041858 | hg38 |
| chr1 | 237042179 | 237042192 | hg38 |
| chr1 | 243482942 | 243483452 | hg38 |
| chr2 | 176068649 | 176068708 | hg38 |
| chr2 | 468079 | 468609 | hg38 |
| chr2 | 5693692 | 5693711 | hg38 |
| chr2 | 10793123 | 10793124 | hg38 |
| chr2 | 19351547 | 19351588 | hg38 |
| chr2 | 19351591 | 19351631 | hg38 |
| chr2 | 19351975 | 19352029 | hg38 |
| chr2 | 19352175 | 19352193 | hg38 |
| chr2 | 19355942 | 19356006 | hg38 |
| chr2 | 19356582 | 19356602 | hg38 |
| chr2 | 19356741 | 19356763 | hg38 |
| chr2 | 19356800 | 19356826 | hg38 |
| chr2 | 19356936 | 19356954 | hg38 |
| chr2 | 19357115 | 19357153 | hg38 |
| chr2 | 19361282 | 19361300 | hg38 |
| chr2 | 27049978 | 27050532 | hg38 |
| chr2 | 38074678 | 38074696 | hg38 |
| chr2 | 38074724 | 38074741 | hg38 |
| chr2 | 38074788 | 38074805 | hg38 |
| chr2 | 38074824 | 38074840 | hg38 |
| chr2 | 38074841 | 38074868 | hg38 |
| chr2 | 38074872 | 38074891 | hg38 |
| chr2 | 43974237 | 43974238 | hg38 |
| chr2 | 44927101 | 44927142 | hg38 |
| chr2 | 44928062 | 44928079 | hg38 |
| chr2 | 44928120 | 44928147 | hg38 |
| chr2 | 44928155 | 44928167 | hg38 |
| chr2 | 44928967 | 44928993 | hg38 |
| chr2 | 44929086 | 44929110 | hg38 |
| chr2 | 44929712 | 44929802 | hg38 |
| chr2 | 44930144 | 44930156 | hg38 |
| chr2 | 44930240 | 44930440 | hg38 |
| chr2 | 44930480 | 44930494 | hg38 |
| chr2 | 44932295 | 44932309 | hg38 |
| chr2 | 44932755 | 44932766 | hg38 |
| chr2 | 44932906 | 44932919 | hg38 |
| chr2 | 44939835 | 44939882 | hg38 |
| chr2 | 44942540 | 44942636 | hg38 |
| chr2 | 44942645 | 44942652 | hg38 |
| chr2 | 44942697 | 44942723 | hg38 |
| chr2 | 44942876 | 44942894 | hg38 |
| chr2 | 44943051 | 44943061 | hg38 |
| chr2 | 44943411 | 44943469 | hg38 |
| chr2 | 44944285 | 44944330 | hg38 |
| chr2 | 44944426 | 44944509 | hg38 |
| chr2 | 44944542 | 44944549 | hg38 |
| chr2 | 44944595 | 44944614 | hg38 |
| chr2 | 44944648 | 44944694 | hg38 |
| chr2 | 44945011 | 44945127 | hg38 |
| chr2 | 44954519 | 44954549 | hg38 |
| chr2 | 45000510 | 45000518 | hg38 |
| chr2 | 45000565 | 45000579 | hg38 |
| chr2 | 45000685 | 45000741 | hg38 |
| chr2 | 45000753 | 45000786 | hg38 |
| chr2 | 45000821 | 45000850 | hg38 |
| chr2 | 45000879 | 45000900 | hg38 |
| chr2 | 45000962 | 45001795 | hg38 |
| chr2 | 45003972 | 45004048 | hg38 |
| chr2 | 45004073 | 45004088 | hg38 |
| chr2 | 45004114 | 45004129 | hg38 |
| chr2 | 45004140 | 45004163 | hg38 |
| chr2 | 45004565 | 45004586 | hg38 |
| chr2 | 45004752 | 45004811 | hg38 |
| chr2 | 45005083 | 45005116 | hg38 |
| chr2 | 45005278 | 45005303 | hg38 |
| chr2 | 45005354 | 45005373 | hg38 |
| chr2 | 45006048 | 45006098 | hg38 |
| chr2 | 45006262 | 45006297 | hg38 |
| chr2 | 45006323 | 45006359 | hg38 |
| chr2 | 45006433 | 45006476 | hg38 |
| chr2 | 45008568 | 45008585 | hg38 |
| chr2 | 45012868 | 45012911 | hg38 |
| chr2 | 45013234 | 45013326 | hg38 |
| chr2 | 45170965 | 45170990 | hg38 |
| chr2 | 45170998 | 45171022 | hg38 |
| chr2 | 53899904 | 53899905 | hg38 |
| chr2 | 63047672 | 63047690 | hg38 |
| chr2 | 63048427 | 63048468 | hg38 |
| chr2 | 63048659 | 63048683 | hg38 |
| chr2 | 63048849 | 63048876 | hg38 |
| chr2 | 63051981 | 63052010 | hg38 |
| chr2 | 63052133 | 63052152 | hg38 |
| chr2 | 63052286 | 63052361 | hg38 |
| chr2 | 63052844 | 63052868 | hg38 |
| chr2 | 63052894 | 63053075 | hg38 |
| chr2 | 63053259 | 63053304 | hg38 |
| chr2 | 63053323 | 63053378 | hg38 |
| chr2 | 63053889 | 63054159 | hg38 |
| chr2 | 63054430 | 63054443 | hg38 |
| chr2 | 63055521 | 63055549 | hg38 |
| chr2 | 63055607 | 63055690 | hg38 |
| chr2 | 63055700 | 63055734 | hg38 |
| chr2 | 63055751 | 63055848 | hg38 |
| chr2 | 63056343 | 63056409 | hg38 |
| chr2 | 63057649 | 63057690 | hg38 |
| chr2 | 63058223 | 63058520 | hg38 |
| chr2 | 63058700 | 63058738 | hg38 |
| chr2 | 63058815 | 63058830 | hg38 |
| chr2 | 63058839 | 63058856 | hg38 |
| chr2 | 63058871 | 63058915 | hg38 |
| chr2 | 63058995 | 63059064 | hg38 |
| chr2 | 63059070 | 63059094 | hg38 |
| chr2 | 63059201 | 63059224 | hg38 |
| chr2 | 63059344 | 63059367 | hg38 |
| chr2 | 63059416 | 63059513 | hg38 |
| chr2 | 63059549 | 63059560 | hg38 |
| chr2 | 66581302 | 66582416 | hg38 |
| chr2 | 68161691 | 68161692 | hg38 |
| chr2 | 70888848 | 70888865 | hg38 |
| chr2 | 70889255 | 70889274 | hg38 |
| chr2 | 70889530 | 70889569 | hg38 |
| chr2 | 70889579 | 70889607 | hg38 |
| chr2 | 70897608 | 70897644 | hg38 |
| chr2 | 70904864 | 70904879 | hg38 |
| chr2 | 70904927 | 70904952 | hg38 |
| chr2 | 70905026 | 70905062 | hg38 |
| chr2 | 70905821 | 70905872 | hg38 |
| chr2 | 70907195 | 70907282 | hg38 |
| chr2 | 70907296 | 70907325 | hg38 |
| chr2 | 70907361 | 70907395 | hg38 |
| chr2 | 70907478 | 70907527 | hg38 |
| chr2 | 70907596 | 70907662 | hg38 |
| chr2 | 70943677 | 70943678 | hg38 |
| chr2 | 72920149 | 72920157 | hg38 |
| chr2 | 72920276 | 72920956 | hg38 |
| chr2 | 74499355 | 74499367 | hg38 |
| chr2 | 74499522 | 74499584 | hg38 |
| chr2 | 74516576 | 74516592 | hg38 |
| chr2 | 74554939 | 74555096 | hg38 |
| chr2 | 80302565 | 80302590 | hg38 |
| chr2 | 80302730 | 80302752 | hg38 |
| chr2 | 80303212 | 80303652 | hg38 |
| chr2 | 80322640 | 80322646 | hg38 |
| chr2 | 85135383 | 85135404 | hg38 |
| chr2 | 94735854 | 94735878 | hg38 |
| chr2 | 95025153 | 95025165 | hg38 |
| chr2 | 96947879 | 96947880 | hg38 |
| chr2 | 98347058 | 98347064 | hg38 |
| chr2 | 98347551 | 98347557 | hg38 |
| chr2 | 98347698 | 98347714 | hg38 |
| chr2 | 98937253 | 98937280 | hg38 |
| chr2 | 104842579 | 104843099 | hg38 |
| chr2 | 104843109 | 104843425 | hg38 |
| chr2 | 104843851 | 104843859 | hg38 |
| chr2 | 104856291 | 104856302 | hg38 |
| chr2 | 104856407 | 104856413 | hg38 |
| chr2 | 104856504 | 104856534 | hg38 |
| chr2 | 104856715 | 104856739 | hg38 |
| chr2 | 104856758 | 104856770 | hg38 |
| chr2 | 104857981 | 104858015 | hg38 |
| chr2 | 104862147 | 104862159 | hg38 |
| chr2 | 104863971 | 104864004 | hg38 |
| chr2 | 104864146 | 104864177 | hg38 |
| chr2 | 104864233 | 104864261 | hg38 |
| chr2 | 106065416 | 106065866 | hg38 |
| chr2 | 111118735 | 111118835 | hg38 |
| chr2 | 111118871 | 111118905 | hg38 |
| chr2 | 111118925 | 111118949 | hg38 |
| chr2 | 111119147 | 111119159 | hg38 |
| chr2 | 111119168 | 111119179 | hg38 |
| chr2 | 111119370 | 111119381 | hg38 |
| chr2 | 113275822 | 113275878 | hg38 |
| chr2 | 113275948 | 113275953 | hg38 |
| chr2 | 113276004 | 113276021 | hg38 |
| chr2 | 113276717 | 113276763 | hg38 |
| chr2 | 113277505 | 113277535 | hg38 |
| chr2 | 113277568 | 113277590 | hg38 |
| chr2 | 113277595 | 113277603 | hg38 |
| chr2 | 113277740 | 113277773 | hg38 |
| chr2 | 113277791 | 113277805 | hg38 |
| chr2 | 113277843 | 113277867 | hg38 |
| chr2 | 113278254 | 113278314 | hg38 |
| chr2 | 113278328 | 113278334 | hg38 |
| chr2 | 113499337 | 113499427 | hg38 |
| chr2 | 113499458 | 113499978 | hg38 |
| chr2 | 118224344 | 118224371 | hg38 |
| chr2 | 118224378 | 118224523 | hg38 |
| chr2 | 118224539 | 118224672 | hg38 |
| chr2 | 118224806 | 118224820 | hg38 |
| chr2 | 118842366 | 118842427 | hg38 |
| chr2 | 118842901 | 118842926 | hg38 |
| chr2 | 118849816 | 118849855 | hg38 |
| chr2 | 118850099 | 118850155 | hg38 |
| chr2 | 118852842 | 118852849 | hg38 |
| chr2 | 118853365 | 118853380 | hg38 |
| chr2 | 118857544 | 118857695 | hg38 |
| chr2 | 118857937 | 118857984 | hg38 |
| chr2 | 118858755 | 118858830 | hg38 |
| chr2 | 118858984 | 118858993 | hg38 |
| chr2 | 127664155 | 127664385 | hg38 |
| chr2 | 154699372 | 154699405 | hg38 |
| chr2 | 154699450 | 154699491 | hg38 |
| chr2 | 156319942 | 156319972 | hg38 |
| chr2 | 156319998 | 156320037 | hg38 |
| chr2 | 156320062 | 156320114 | hg38 |
| chr2 | 156320248 | 156320256 | hg38 |
| chr2 | 156320278 | 156320301 | hg38 |
| chr2 | 156320459 | 156320508 | hg38 |
| chr2 | 156320555 | 156320563 | hg38 |
| chr2 | 156322830 | 156322918 | hg38 |
| chr2 | 156322931 | 156322939 | hg38 |
| chr2 | 156329156 | 156329218 | hg38 |
| chr2 | 156329505 | 156329610 | hg38 |
| chr2 | 156329803 | 156329843 | hg38 |
| chr2 | 161423394 | 161423415 | hg38 |
| chr2 | 161424606 | 161424674 | hg38 |
| chr2 | 161426411 | 161426461 | hg38 |
| chr2 | 161426632 | 161426652 | hg38 |
| chr2 | 161427152 | 161427211 | hg38 |
| chr2 | 161427300 | 161427346 | hg38 |
| chr2 | 161427382 | 161427407 | hg38 |
| chr2 | 161427483 | 161427531 | hg38 |
| chr2 | 161427843 | 161427929 | hg38 |
| chr2 | 161428009 | 161428068 | hg38 |
| chr2 | 170822188 | 170822230 | hg38 |
| chr2 | 170822657 | 170822669 | hg38 |
| chr2 | 170822888 | 170823388 | hg38 |
| chr2 | 170823418 | 170823708 | hg38 |
| chr2 | 172080269 | 172080294 | hg38 |
| chr2 | 172080330 | 172080341 | hg38 |
| chr2 | 172080416 | 172080426 | hg38 |
| chr2 | 172080507 | 172080533 | hg38 |
| chr2 | 172080546 | 172080599 | hg38 |
| chr2 | 172080600 | 172080636 | hg38 |
| chr2 | 172080968 | 172080995 | hg38 |
| chr2 | 172081273 | 172081406 | hg38 |
| chr2 | 172087401 | 172087411 | hg38 |
| chr2 | 174328733 | 174328806 | hg38 |
| chr2 | 174328942 | 174329027 | hg38 |
| chr2 | 174340836 | 174340906 | hg38 |
| chr2 | 174343731 | 174343745 | hg38 |
| chr2 | 174343945 | 174343975 | hg38 |
| chr2 | 174344233 | 174344270 | hg38 |
| chr2 | 174344326 | 174344333 | hg38 |
| chr2 | 176066848 | 176066965 | hg38 |
| chr2 | 176067228 | 176067241 | hg38 |
| chr2 | 176067253 | 176067270 | hg38 |
| chr2 | 176067865 | 176067883 | hg38 |
| chr2 | 176067894 | 176067931 | hg38 |
| chr2 | 176068053 | 176068091 | hg38 |
| chr2 | 176068601 | 176068626 | hg38 |
| chr2 | 176071577 | 176071681 | hg38 |
| chr2 | 176071752 | 176071763 | hg38 |
| chr2 | 176071786 | 176071816 | hg38 |
| chr2 | 176071843 | 176071908 | hg38 |
| chr2 | 176072186 | 176072215 | hg38 |
| chr2 | 176080822 | 176080858 | hg38 |
| chr2 | 176081772 | 176081899 | hg38 |
| chr2 | 176081970 | 176082142 | hg38 |
| chr2 | 176082210 | 176082225 | hg38 |
| chr2 | 176082227 | 176082252 | hg38 |
| chr2 | 176082276 | 176082288 | hg38 |
| chr2 | 176082302 | 176082334 | hg38 |
| chr2 | 176082346 | 176082391 | hg38 |
| chr2 | 176082403 | 176082413 | hg38 |
| chr2 | 176082430 | 176082445 | hg38 |
| chr2 | 176082516 | 176082551 | hg38 |
| chr2 | 176083067 | 176083086 | hg38 |
| chr2 | 176091807 | 176091846 | hg38 |
| chr2 | 176091903 | 176091972 | hg38 |
| chr2 | 176092102 | 176092230 | hg38 |
| chr2 | 176099409 | 176099441 | hg38 |
| chr2 | 176099447 | 176099569 | hg38 |
| chr2 | 176099662 | 176099729 | hg38 |
| chr2 | 176099778 | 176099843 | hg38 |
| chr2 | 176099893 | 176099915 | hg38 |
| chr2 | 176099937 | 176099993 | hg38 |
| chr2 | 176100135 | 176100159 | hg38 |
| chr2 | 176100734 | 176100763 | hg38 |
| chr2 | 176112591 | 176112625 | hg38 |
| chr2 | 176116555 | 176116601 | hg38 |
| chr2 | 176116616 | 176116765 | hg38 |
| chr2 | 176121761 | 176121804 | hg38 |
| chr2 | 176123946 | 176123973 | hg38 |
| chr2 | 176128240 | 176128247 | hg38 |
| chr2 | 176128860 | 176128905 | hg38 |
| chr2 | 176132711 | 176132759 | hg38 |
| chr2 | 176147673 | 176147701 | hg38 |
| chr2 | 176150231 | 176150239 | hg38 |
| chr2 | 176151116 | 176151135 | hg38 |
| chr2 | 176151689 | 176151748 | hg38 |
| chr2 | 176160830 | 176160838 | hg38 |
| chr2 | 176161247 | 176161271 | hg38 |
| chr2 | 176162682 | 176162713 | hg38 |
| chr2 | 176164700 | 176164744 | hg38 |
| chr2 | 176164907 | 176164928 | hg38 |
| chr2 | 176171636 | 176171677 | hg38 |
| chr2 | 176171681 | 176171712 | hg38 |
| chr2 | 176171868 | 176171882 | hg38 |
| chr2 | 176172275 | 176172283 | hg38 |
| chr2 | 181456691 | 181456815 | hg38 |
| chr2 | 181678356 | 181678400 | hg38 |
| chr2 | 181678501 | 181678530 | hg38 |
| chr2 | 181678535 | 181678569 | hg38 |
| chr2 | 181683206 | 181683212 | hg38 |
| chr2 | 190360255 | 190360256 | hg38 |
| chr2 | 199461889 | 199461962 | hg38 |
| chr2 | 199462138 | 199462202 | hg38 |
| chr2 | 199462331 | 199462343 | hg38 |
| chr2 | 199462355 | 199462413 | hg38 |
| chr2 | 199462517 | 199462545 | hg38 |
| chr2 | 199464333 | 199464429 | hg38 |
| chr2 | 199464585 | 199464617 | hg38 |
| chr2 | 199464686 | 199464694 | hg38 |
| chr2 | 199464697 | 199464786 | hg38 |
| chr2 | 199468997 | 199469012 | hg38 |
| chr2 | 199469068 | 199469073 | hg38 |
| chr2 | 199470257 | 199470272 | hg38 |
| chr2 | 199470775 | 199470800 | hg38 |
| chr2 | 199470936 | 199471085 | hg38 |
| chr2 | 201160898 | 201160899 | hg38 |
| chr2 | 206642134 | 206642145 | hg38 |
| chr2 | 206642443 | 206642478 | hg38 |
| chr2 | 215023900 | 215023901 | hg38 |
| chr2 | 218870950 | 218872020 | hg38 |
| chr2 | 218873440 | 218873478 | hg38 |
| chr2 | 218873605 | 218873612 | hg38 |
| chr2 | 218873774 | 218873808 | hg38 |
| chr2 | 222289421 | 222289451 | hg38 |
| chr2 | 222289453 | 222289471 | hg38 |
| chr2 | 222294286 | 222294426 | hg38 |
| chr2 | 222297058 | 222297079 | hg38 |
| chr2 | 222297080 | 222297097 | hg38 |
| chr2 | 222298324 | 222298349 | hg38 |
| chr2 | 222298507 | 222298513 | hg38 |
| chr2 | 222298538 | 222298554 | hg38 |
| chr2 | 222298670 | 222298680 | hg38 |
| chr2 | 222300148 | 222300172 | hg38 |
| chr2 | 222301296 | 222301376 | hg38 |
| chr2 | 222301644 | 222301788 | hg38 |
| chr2 | 222302799 | 222302818 | hg38 |
| chr2 | 222304722 | 222304783 | hg38 |
| chr2 | 222308221 | 222308350 | hg38 |
| chr2 | 222312349 | 222312487 | hg38 |
| chr2 | 236169554 | 236169620 | hg38 |
| chr2 | 236170967 | 236170988 | hg38 |
| chr2 | 238846552 | 238846575 | hg38 |
| chr2 | 238846605 | 238846624 | hg38 |
| chr2 | 241849249 | 241849282 | hg38 |
| chr3 | 5096078 | 5096111 | hg38 |
| chr3 | 5096166 | 5096175 | hg38 |
| chr3 | 9136266 | 9136281 | hg38 |
| chr3 | 9365152 | 9365153 | hg38 |
| chr3 | 21265909 | 21265910 | hg38 |
| chr3 | 27724134 | 27724151 | hg38 |
| chr3 | 27724741 | 27724769 | hg38 |
| chr3 | 27724812 | 27724831 | hg38 |
| chr3 | 33393339 | 33393340 | hg38 |
| chr3 | 61564452 | 61564500 | hg38 |
| chr3 | 62367653 | 62367899 | hg38 |
| chr3 | 62368179 | 62368285 | hg38 |
| chr3 | 62369770 | 62369790 | hg38 |
| chr3 | 62370533 | 62370549 | hg38 |
| chr3 | 62371253 | 62371272 | hg38 |
| chr3 | 62377293 | 62377315 | hg38 |
| chr3 | 62377366 | 62377394 | hg38 |
| chr3 | 128076033 | 128076114 | hg38 |
| chr3 | 128076796 | 128077266 | hg38 |
| chr3 | 129974349 | 129975054 | hg38 |
| chr3 | 129975144 | 129975704 | hg38 |
| chr3 | 130345826 | 130345843 | hg38 |
| chr3 | 130517296 | 130517306 | hg38 |
| chr3 | 137772104 | 137772128 | hg38 |
| chr3 | 138936782 | 138936787 | hg38 |
| chr3 | 138937164 | 138937190 | hg38 |
| chr3 | 138938455 | 138938675 | hg38 |
| chr3 | 138939045 | 138939305 | hg38 |
| chr3 | 138939575 | 138939894 | hg38 |
| chr3 | 138940046 | 138940180 | hg38 |
| chr3 | 138940237 | 138940244 | hg38 |
| chr3 | 138943308 | 138943340 | hg38 |
| chr3 | 138943523 | 138943573 | hg38 |
| chr3 | 138943973 | 138943994 | hg38 |
| chr3 | 141797263 | 141797313 | hg38 |
| chr3 | 141797678 | 141797730 | hg38 |
| chr3 | 147359571 | 147359577 | hg38 |
| chr3 | 147359814 | 147359867 | hg38 |
| chr3 | 147388238 | 147388290 | hg38 |
| chr3 | 147388799 | 147388842 | hg38 |
| chr3 | 147390725 | 147390746 | hg38 |
| chr3 | 147390839 | 147390846 | hg38 |
| chr3 | 147390887 | 147390958 | hg38 |
| chr3 | 147391040 | 147391071 | hg38 |
| chr3 | 147391129 | 147391200 | hg38 |
| chr3 | 147391204 | 147391227 | hg38 |
| chr3 | 147391980 | 147392024 | hg38 |
| chr3 | 147392794 | 147392925 | hg38 |
| chr3 | 147393612 | 147393638 | hg38 |
| chr3 | 147393823 | 147393840 | hg38 |
| chr3 | 147395828 | 147395842 | hg38 |
| chr3 | 147395951 | 147396063 | hg38 |
| chr3 | 147396117 | 147396139 | hg38 |
| chr3 | 147396434 | 147396518 | hg38 |
| chr3 | 147396519 | 147396559 | hg38 |
| chr3 | 147396812 | 147396862 | hg38 |
| chr3 | 147397161 | 147397198 | hg38 |
| chr3 | 147397256 | 147397290 | hg38 |
| chr3 | 147398351 | 147398391 | hg38 |
| chr3 | 147398557 | 147398625 | hg38 |
| chr3 | 147405686 | 147405691 | hg38 |
| chr3 | 147405935 | 147405954 | hg38 |
| chr3 | 147408137 | 147408156 | hg38 |
| chr3 | 147409202 | 147409229 | hg38 |
| chr3 | 147409236 | 147409282 | hg38 |
| chr3 | 147409630 | 147409662 | hg38 |
| chr3 | 147409693 | 147409705 | hg38 |
| chr3 | 147409796 | 147409823 | hg38 |
| chr3 | 147409832 | 147409883 | hg38 |
| chr3 | 147409889 | 147409911 | hg38 |
| chr3 | 147409930 | 147409967 | hg38 |
| chr3 | 147410127 | 147410149 | hg38 |
| chr3 | 147410168 | 147410187 | hg38 |
| chr3 | 147410199 | 147410209 | hg38 |
| chr3 | 147410270 | 147410290 | hg38 |
| chr3 | 147410306 | 147410326 | hg38 |
| chr3 | 147410367 | 147410420 | hg38 |
| chr3 | 147410425 | 147410440 | hg38 |
| chr3 | 147410441 | 147410488 | hg38 |
| chr3 | 147410516 | 147410526 | hg38 |
| chr3 | 147410573 | 147410584 | hg38 |
| chr3 | 147410615 | 147410637 | hg38 |
| chr3 | 147410665 | 147410685 | hg38 |
| chr3 | 147410736 | 147410765 | hg38 |
| chr3 | 147410883 | 147410904 | hg38 |
| chr3 | 147411051 | 147411056 | hg38 |
| chr3 | 147411417 | 147411433 | hg38 |
| chr3 | 147411987 | 147412014 | hg38 |
| chr3 | 147412576 | 147412668 | hg38 |
| chr3 | 147412929 | 147412980 | hg38 |
| chr3 | 147413370 | 147413392 | hg38 |
| chr3 | 147418905 | 147418943 | hg38 |
| chr3 | 147419175 | 147419207 | hg38 |
| chr3 | 147419420 | 147419470 | hg38 |
| chr3 | 147420636 | 147420676 | hg38 |
| chr3 | 147420832 | 147420878 | hg38 |
| chr3 | 147421383 | 147421390 | hg38 |
| chr3 | 147421464 | 147421492 | hg38 |
| chr3 | 147421614 | 147421649 | hg38 |
| chr3 | 147421727 | 147421771 | hg38 |
| chr3 | 147422331 | 147422358 | hg38 |
| chr3 | 147423040 | 147423094 | hg38 |
| chr3 | 147423098 | 147423146 | hg38 |
| chr3 | 147423337 | 147423377 | hg38 |
| chr3 | 147423427 | 147423473 | hg38 |
| chr3 | 147423485 | 147423502 | hg38 |
| chr3 | 147424395 | 147424418 | hg38 |
| chr3 | 147424543 | 147424569 | hg38 |
| chr3 | 154428360 | 154428383 | hg38 |
| chr3 | 156291440 | 156291461 | hg38 |
| chr3 | 156291505 | 156291519 | hg38 |
| chr3 | 158094423 | 158094499 | hg38 |
| chr3 | 158094513 | 158094570 | hg38 |
| chr3 | 158094615 | 158094687 | hg38 |
| chr3 | 158094828 | 158094900 | hg38 |
| chr3 | 158095200 | 158095229 | hg38 |
| chr3 | 158102685 | 158102753 | hg38 |
| chr3 | 158102852 | 158102880 | hg38 |
| chr3 | 158103274 | 158103315 | hg38 |
| chr3 | 158103343 | 158103445 | hg38 |
| chr3 | 158103457 | 158103599 | hg38 |
| chr3 | 158103618 | 158103730 | hg38 |
| chr3 | 158103739 | 158103808 | hg38 |
| chr3 | 158104236 | 158104245 | hg38 |
| chr3 | 169659976 | 169660035 | hg38 |
| chr3 | 170419644 | 170419655 | hg38 |
| chr3 | 170419669 | 170419681 | hg38 |
| chr3 | 170419782 | 170419846 | hg38 |
| chr3 | 170419885 | 170419927 | hg38 |
| chr3 | 170419935 | 170419971 | hg38 |
| chr3 | 170419976 | 170419997 | hg38 |
| chr3 | 170420014 | 170420077 | hg38 |
| chr3 | 170585256 | 170585280 | hg38 |
| chr3 | 170585583 | 170585606 | hg38 |
| chr3 | 170585615 | 170585625 | hg38 |
| chr3 | 172447603 | 172447631 | hg38 |
| chr3 | 172447797 | 172447844 | hg38 |
| chr3 | 172447855 | 172447892 | hg38 |
| chr3 | 172447965 | 172448051 | hg38 |
| chr3 | 172448149 | 172448252 | hg38 |
| chr3 | 172448320 | 172448333 | hg38 |
| chr3 | 172448402 | 172448420 | hg38 |
| chr3 | 172448790 | 172448824 | hg38 |
| chr3 | 172448839 | 172448847 | hg38 |
| chr3 | 181695617 | 181695685 | hg38 |
| chr3 | 181723467 | 181723577 | hg38 |
| chr3 | 181723713 | 181723745 | hg38 |
| chr3 | 181723757 | 181723825 | hg38 |
| chr3 | 181724000 | 181724048 | hg38 |
| chr3 | 181724357 | 181724391 | hg38 |
| chr3 | 181724414 | 181724457 | hg38 |
| chr3 | 181724888 | 181724926 | hg38 |
| chr3 | 181725215 | 181725305 | hg38 |
| chr3 | 187670246 | 187670279 | hg38 |
| chr3 | 192408262 | 192408282 | hg38 |
| chr3 | 194397952 | 194398259 | hg38 |
| chr3 | 196640607 | 196640637 | hg38 |
| chr3 | 198113885 | 198113895 | hg38 |
| chr4 | 107393 | 107416 | hg38 |
| chr4 | 212819 | 212877 | hg38 |
| chr4 | 386368 | 386382 | hg38 |
| chr4 | 386420 | 386479 | hg38 |
| chr4 | 386583 | 386592 | hg38 |
| chr4 | 967779 | 967786 | hg38 |
| chr4 | 1403283 | 1403350 | hg38 |
| chr4 | 1405896 | 1405936 | hg38 |
| chr4 | 1406254 | 1406276 | hg38 |
| chr4 | 1406838 | 1406866 | hg38 |
| chr4 | 3767061 | 3767075 | hg38 |
| chr4 | 4862521 | 4862569 | hg38 |
| chr4 | 5892109 | 5892117 | hg38 |
| chr4 | 8891664 | 8891678 | hg38 |
| chr4 | 10461282 | 10461333 | hg38 |
| chr4 | 10461543 | 10461573 | hg38 |
| chr4 | 10461966 | 10461977 | hg38 |
| chr4 | 13522258 | 13522968 | hg38 |
| chr4 | 13523418 | 13523475 | hg38 |
| chr4 | 13523481 | 13523508 | hg38 |
| chr4 | 13523512 | 13523560 | hg38 |
| chr4 | 13523896 | 13523965 | hg38 |
| chr4 | 13524081 | 13524146 | hg38 |
| chr4 | 13537399 | 13537422 | hg38 |
| chr4 | 13541223 | 13541273 | hg38 |
| chr4 | 13542775 | 13542849 | hg38 |
| chr4 | 13542946 | 13542959 | hg38 |
| chr4 | 13543182 | 13543229 | hg38 |
| chr4 | 13543644 | 13543664 | hg38 |
| chr4 | 13543931 | 13543956 | hg38 |
| chr4 | 24799784 | 24799792 | hg38 |
| chr4 | 24799829 | 24799855 | hg38 |
| chr4 | 24799863 | 24799906 | hg38 |
| chr4 | 24799922 | 24799941 | hg38 |
| chr4 | 24799949 | 24800023 | hg38 |
| chr4 | 24800024 | 24800053 | hg38 |
| chr4 | 24800086 | 24800092 | hg38 |
| chr4 | 24800121 | 24800187 | hg38 |
| chr4 | 24800215 | 24800249 | hg38 |
| chr4 | 41745072 | 41745079 | hg38 |
| chr4 | 41746010 | 41746018 | hg38 |
| chr4 | 41747479 | 41747508 | hg38 |
| chr4 | 41747739 | 41747784 | hg38 |
| chr4 | 41751264 | 41751269 | hg38 |
| chr4 | 41751287 | 41751307 | hg38 |
| chr4 | 41866511 | 41866541 | hg38 |
| chr4 | 41873457 | 41873507 | hg38 |
| chr4 | 41879332 | 41879385 | hg38 |
| chr4 | 41880097 | 41880145 | hg38 |
| chr4 | 41880146 | 41880157 | hg38 |
| chr4 | 41880198 | 41880208 | hg38 |
| chr4 | 41880434 | 41880456 | hg38 |
| chr4 | 41880466 | 41880500 | hg38 |
| chr4 | 46993693 | 46993698 | hg38 |
| chr4 | 46993716 | 46993725 | hg38 |
| chr4 | 48490818 | 48490832 | hg38 |
| chr4 | 48491289 | 48491442 | hg38 |
| chr4 | 48491458 | 48491475 | hg38 |
| chr4 | 54104295 | 54104325 | hg38 |
| chr4 | 54108491 | 54108501 | hg38 |
| chr4 | 54108993 | 54109057 | hg38 |
| chr4 | 54109240 | 54109262 | hg38 |
| chr4 | 56655340 | 56655416 | hg38 |
| chr4 | 56655514 | 56655568 | hg38 |
| chr4 | 80268665 | 80268697 | hg38 |
| chr4 | 80268723 | 80268744 | hg38 |
| chr4 | 84481290 | 84481339 | hg38 |
| chr4 | 84481633 | 84481648 | hg38 |
| chr4 | 84481669 | 84481725 | hg38 |
| chr4 | 84481817 | 84482071 | hg38 |
| chr4 | 84483610 | 84483654 | hg38 |
| chr4 | 84483834 | 84483907 | hg38 |
| chr4 | 84495966 | 84496007 | hg38 |
| chr4 | 84496048 | 84496060 | hg38 |
| chr4 | 84496138 | 84496200 | hg38 |
| chr4 | 84496662 | 84496681 | hg38 |
| chr4 | 84496902 | 84496917 | hg38 |
| chr4 | 93834648 | 93834684 | hg38 |
| chr4 | 101345939 | 101346163 | hg38 |
| chr4 | 110619796 | 110619829 | hg38 |
| chr4 | 110629354 | 110629379 | hg38 |
| chr4 | 110629462 | 110629490 | hg38 |
| chr4 | 110629674 | 110629692 | hg38 |
| chr4 | 110637880 | 110637950 | hg38 |
| chr4 | 110641490 | 110641521 | hg38 |
| chr4 | 125319355 | 125319356 | hg38 |
| chr4 | 139279310 | 139279840 | hg38 |
| chr4 | 139735800 | 139735810 | hg38 |
| chr4 | 139735857 | 139735866 | hg38 |
| chr4 | 146638976 | 146638993 | hg38 |
| chr4 | 146639320 | 146639327 | hg38 |
| chr4 | 146639329 | 146639336 | hg38 |
| chr4 | 146639806 | 146639837 | hg38 |
| chr4 | 146640364 | 146640424 | hg38 |
| chr4 | 146640569 | 146640605 | hg38 |
| chr4 | 146640639 | 146640655 | hg38 |
| chr4 | 153792390 | 153792910 | hg38 |
| chr4 | 154741355 | 154741377 | hg38 |
| chr4 | 154741717 | 154742770 | hg38 |
| chr4 | 173500295 | 173500341 | hg38 |
| chr4 | 173500358 | 173500376 | hg38 |
| chr4 | 173500889 | 173500904 | hg38 |
| chr4 | 173501012 | 173501079 | hg38 |
| chr4 | 173501164 | 173501185 | hg38 |
| chr4 | 173501205 | 173501251 | hg38 |
| chr4 | 173508516 | 173508540 | hg38 |
| chr4 | 173509240 | 173509245 | hg38 |
| chr4 | 173509433 | 173509893 | hg38 |
| chr4 | 173516799 | 173516811 | hg38 |
| chr4 | 173518839 | 173518929 | hg38 |
| chr4 | 173523301 | 173523348 | hg38 |
| chr4 | 173528433 | 173528446 | hg38 |
| chr4 | 173531684 | 173531754 | hg38 |
| chr4 | 173537853 | 173538223 | hg38 |
| chr4 | 173538263 | 173538283 | hg38 |
| chr4 | 174213762 | 174213771 | hg38 |
| chr4 | 182142317 | 182142357 | hg38 |
| chr4 | 185016052 | 185016116 | hg38 |
| chr4 | 185016135 | 185016184 | hg38 |
| chr4 | 185190957 | 185190958 | hg38 |
| chr4 | 190018558 | 190018571 | hg38 |
| chr5 | 1876272 | 1876284 | hg38 |
| chr5 | 1876443 | 1876457 | hg38 |
| chr5 | 1877760 | 1877796 | hg38 |
| chr5 | 1877919 | 1877935 | hg38 |
| chr5 | 1878287 | 1878305 | hg38 |
| chr5 | 1878394 | 1878401 | hg38 |
| chr5 | 1878487 | 1878528 | hg38 |
| chr5 | 1878539 | 1878559 | hg38 |
| chr5 | 1878622 | 1878647 | hg38 |
| chr5 | 1878658 | 1878667 | hg38 |
| chr5 | 1878679 | 1878710 | hg38 |
| chr5 | 1879591 | 1879614 | hg38 |
| chr5 | 1879644 | 1879661 | hg38 |
| chr5 | 1879746 | 1879789 | hg38 |
| chr5 | 1879790 | 1879809 | hg38 |
| chr5 | 1881907 | 1881928 | hg38 |
| chr5 | 1881955 | 1881980 | hg38 |
| chr5 | 1882006 | 1882024 | hg38 |
| chr5 | 1885219 | 1885331 | hg38 |
| chr5 | 1885466 | 1885526 | hg38 |
| chr5 | 2738982 | 2739003 | hg38 |
| chr5 | 2740121 | 2740128 | hg38 |
| chr5 | 2740313 | 2740327 | hg38 |
| chr5 | 2740459 | 2740529 | hg38 |
| chr5 | 2740829 | 2740902 | hg38 |
| chr5 | 2741058 | 2741080 | hg38 |
| chr5 | 2741084 | 2741105 | hg38 |
| chr5 | 2748363 | 2748371 | hg38 |
| chr5 | 2748394 | 2748416 | hg38 |
| chr5 | 2748456 | 2748483 | hg38 |
| chr5 | 2748508 | 2748523 | hg38 |
| chr5 | 2748540 | 2748554 | hg38 |
| chr5 | 2748631 | 2748637 | hg38 |
| chr5 | 2748652 | 2748668 | hg38 |
| chr5 | 2748847 | 2748872 | hg38 |
| chr5 | 2748903 | 2748913 | hg38 |
| chr5 | 2748952 | 2748973 | hg38 |
| chr5 | 2749000 | 2749016 | hg38 |
| chr5 | 2749238 | 2749263 | hg38 |
| chr5 | 2749525 | 2749542 | hg38 |
| chr5 | 2749689 | 2749720 | hg38 |
| chr5 | 2749772 | 2749785 | hg38 |
| chr5 | 2749917 | 2749940 | hg38 |
| chr5 | 2755239 | 2755246 | hg38 |
| chr5 | 2755615 | 2755635 | hg38 |
| chr5 | 2755912 | 2755998 | hg38 |
| chr5 | 2757284 | 2757344 | hg38 |
| chr5 | 3590632 | 3590664 | hg38 |
| chr5 | 3592744 | 3592754 | hg38 |
| chr5 | 3594778 | 3594784 | hg38 |
| chr5 | 3596671 | 3596681 | hg38 |
| chr5 | 3596752 | 3596766 | hg38 |
| chr5 | 3596804 | 3596823 | hg38 |
| chr5 | 3597313 | 3597334 | hg38 |
| chr5 | 3597340 | 3597374 | hg38 |
| chr5 | 3597548 | 3597582 | hg38 |
| chr5 | 3597590 | 3597598 | hg38 |
| chr5 | 3599289 | 3599348 | hg38 |
| chr5 | 3599356 | 3599397 | hg38 |
| chr5 | 3599463 | 3599535 | hg38 |
| chr5 | 3599566 | 3599579 | hg38 |
| chr5 | 3599589 | 3599606 | hg38 |
| chr5 | 3599617 | 3599657 | hg38 |
| chr5 | 3599662 | 3599684 | hg38 |
| chr5 | 3599695 | 3599714 | hg38 |
| chr5 | 3599735 | 3599768 | hg38 |
| chr5 | 3599820 | 3599849 | hg38 |
| chr5 | 3599881 | 3599908 | hg38 |
| chr5 | 3599919 | 3599930 | hg38 |
| chr5 | 3599959 | 3599975 | hg38 |
| chr5 | 3599976 | 3599993 | hg38 |
| chr5 | 3599997 | 3600014 | hg38 |
| chr5 | 3600183 | 3600197 | hg38 |
| chr5 | 3601892 | 3601906 | hg38 |
| chr5 | 3602041 | 3602060 | hg38 |
| chr5 | 3606384 | 3606440 | hg38 |
| chr5 | 7395031 | 7395049 | hg38 |
| chr5 | 7395065 | 7395080 | hg38 |
| chr5 | 7395223 | 7395232 | hg38 |
| chr5 | 32709125 | 32709182 | hg38 |
| chr5 | 32712987 | 32712997 | hg38 |
| chr5 | 37834699 | 37834919 | hg38 |
| chr5 | 37836144 | 37836166 | hg38 |
| chr5 | 42952041 | 42952064 | hg38 |
| chr5 | 42993991 | 42994022 | hg38 |
| chr5 | 42994524 | 42994834 | hg38 |
| chr5 | 42995020 | 42995313 | hg38 |
| chr5 | 45695874 | 45695896 | hg38 |
| chr5 | 45696072 | 45696562 | hg38 |
| chr5 | 50966614 | 50966627 | hg38 |
| chr5 | 51377896 | 51377904 | hg38 |
| chr5 | 51378131 | 51378164 | hg38 |
| chr5 | 51389756 | 51389771 | hg38 |
| chr5 | 55222196 | 55222252 | hg38 |
| chr5 | 55222259 | 55222276 | hg38 |
| chr5 | 55223233 | 55223260 | hg38 |
| chr5 | 55223388 | 55223463 | hg38 |
| chr5 | 55223520 | 55223536 | hg38 |
| chr5 | 55223541 | 55223567 | hg38 |
| chr5 | 55223582 | 55223645 | hg38 |
| chr5 | 55223657 | 55223678 | hg38 |
| chr5 | 55223729 | 55223800 | hg38 |
| chr5 | 59893242 | 59893256 | hg38 |
| chr5 | 59893293 | 59893314 | hg38 |
| chr5 | 69123227 | 69123228 | hg38 |
| chr5 | 71719142 | 71719149 | hg38 |
| chr5 | 73230759 | 73230797 | hg38 |
| chr5 | 73230899 | 73230959 | hg38 |
| chr5 | 73232487 | 73232553 | hg38 |
| chr5 | 73232754 | 73232784 | hg38 |
| chr5 | 73297995 | 73298055 | hg38 |
| chr5 | 73298080 | 73298100 | hg38 |
| chr5 | 73298435 | 73298442 | hg38 |
| chr5 | 73298561 | 73298566 | hg38 |
| chr5 | 73380625 | 73380651 | hg38 |
| chr5 | 73380906 | 73380919 | hg38 |
| chr5 | 73381311 | 73381380 | hg38 |
| chr5 | 73381519 | 73381541 | hg38 |
| chr5 | 73382099 | 73382112 | hg38 |
| chr5 | 73382571 | 73382582 | hg38 |
| chr5 | 73436177 | 73436222 | hg38 |
| chr5 | 77627839 | 77628064 | hg38 |
| chr5 | 77628093 | 77628217 | hg38 |
| chr5 | 77628233 | 77628254 | hg38 |
| chr5 | 77628265 | 77628298 | hg38 |
| chr5 | 77628438 | 77628446 | hg38 |
| chr5 | 77628503 | 77628515 | hg38 |
| chr5 | 77628537 | 77628593 | hg38 |
| chr5 | 77632581 | 77632597 | hg38 |
| chr5 | 77636928 | 77636946 | hg38 |
| chr5 | 77638473 | 77638503 | hg38 |
| chr5 | 77639192 | 77639209 | hg38 |
| chr5 | 77639219 | 77639241 | hg38 |
| chr5 | 77646024 | 77646034 | hg38 |
| chr5 | 77972593 | 77972632 | hg38 |
| chr5 | 77972847 | 77972882 | hg38 |
| chr5 | 77973031 | 77973125 | hg38 |
| chr5 | 88674932 | 88674958 | hg38 |
| chr5 | 88678318 | 88678328 | hg38 |
| chr5 | 90525167 | 90525168 | hg38 |
| chr5 | 93570814 | 93570854 | hg38 |
| chr5 | 93587851 | 93587857 | hg38 |
| chr5 | 93602362 | 93602391 | hg38 |
| chr5 | 93602455 | 93602463 | hg38 |
| chr5 | 93602621 | 93602628 | hg38 |
| chr5 | 96790605 | 96790606 | hg38 |
| chr5 | 115816201 | 115817016 | hg38 |
| chr5 | 115962719 | 115962738 | hg38 |
| chr5 | 115963018 | 115963031 | hg38 |
| chr5 | 123086488 | 123086541 | hg38 |
| chr5 | 123086561 | 123086584 | hg38 |
| chr5 | 123086641 | 123086649 | hg38 |
| chr5 | 123087716 | 123087750 | hg38 |
| chr5 | 123094739 | 123094797 | hg38 |
| chr5 | 123095004 | 123095077 | hg38 |
| chr5 | 123095560 | 123095582 | hg38 |
| chr5 | 123095606 | 123095634 | hg38 |
| chr5 | 123098184 | 123098201 | hg38 |
| chr5 | 123098471 | 123098526 | hg38 |
| chr5 | 128537067 | 128537075 | hg38 |
| chr5 | 135028118 | 135028139 | hg38 |
| chr5 | 135028144 | 135028168 | hg38 |
| chr5 | 135028229 | 135028250 | hg38 |
| chr5 | 135028482 | 135028522 | hg38 |
| chr5 | 135028732 | 135028760 | hg38 |
| chr5 | 135028893 | 135029005 | hg38 |
| chr5 | 135029198 | 135029285 | hg38 |
| chr5 | 135038794 | 135038818 | hg38 |
| chr5 | 135040846 | 135040869 | hg38 |
| chr5 | 135040924 | 135040940 | hg38 |
| chr5 | 135040949 | 135040983 | hg38 |
| chr5 | 135050351 | 135050361 | hg38 |
| chr5 | 135050471 | 135050494 | hg38 |
| chr5 | 135050548 | 135050567 | hg38 |
| chr5 | 135050568 | 135050592 | hg38 |
| chr5 | 135490412 | 135490464 | hg38 |
| chr5 | 135491772 | 135491791 | hg38 |
| chr5 | 135534824 | 135534840 | hg38 |
| chr5 | 140787523 | 140787578 | hg38 |
| chr5 | 140787717 | 140787742 | hg38 |
| chr5 | 140787751 | 140787780 | hg38 |
| chr5 | 140787796 | 140787805 | hg38 |
| chr5 | 140787825 | 140787845 | hg38 |
| chr5 | 140787961 | 140787982 | hg38 |
| chr5 | 140788050 | 140788088 | hg38 |
| chr5 | 140788138 | 140788146 | hg38 |
| chr5 | 140788193 | 140788264 | hg38 |
| chr5 | 140788335 | 140788349 | hg38 |
| chr5 | 140788396 | 140788450 | hg38 |
| chr5 | 140828223 | 140828238 | hg38 |
| chr5 | 140882407 | 140882439 | hg38 |
| chr5 | 140927794 | 140927835 | hg38 |
| chr5 | 140967485 | 140967520 | hg38 |
| chr5 | 141151924 | 141151959 | hg38 |
| chr5 | 141152099 | 141152145 | hg38 |
| chr5 | 141188906 | 141188927 | hg38 |
| chr5 | 141188934 | 141188963 | hg38 |
| chr5 | 141189196 | 141189221 | hg38 |
| chr5 | 141194687 | 141194729 | hg38 |
| chr5 | 141225058 | 141225112 | hg38 |
| chr5 | 141225113 | 141225140 | hg38 |
| chr5 | 141225629 | 141225668 | hg38 |
| chr5 | 141236196 | 141236246 | hg38 |
| chr5 | 141241504 | 141241582 | hg38 |
| chr5 | 141241623 | 141241649 | hg38 |
| chr5 | 141361680 | 141361690 | hg38 |
| chr5 | 141361755 | 141361771 | hg38 |
| chr5 | 141370484 | 141370509 | hg38 |
| chr5 | 141408430 | 141408468 | hg38 |
| chr5 | 141409749 | 141409757 | hg38 |
| chr5 | 141409780 | 141409825 | hg38 |
| chr5 | 141409876 | 141409921 | hg38 |
| chr5 | 141410066 | 141410086 | hg38 |
| chr5 | 141410101 | 141410135 | hg38 |
| chr5 | 141418051 | 141418067 | hg38 |
| chr5 | 141418068 | 141418094 | hg38 |
| chr5 | 141419215 | 141419275 | hg38 |
| chr5 | 141419358 | 141419386 | hg38 |
| chr5 | 141419435 | 141419465 | hg38 |
| chr5 | 141419527 | 141419585 | hg38 |
| chr5 | 141419644 | 141419653 | hg38 |
| chr5 | 141419974 | 141420006 | hg38 |
| chr5 | 141421202 | 141421249 | hg38 |
| chr5 | 141421414 | 141421469 | hg38 |
| chr5 | 141422865 | 141422911 | hg38 |
| chr5 | 141422968 | 141423009 | hg38 |
| chr5 | 141423081 | 141423119 | hg38 |
| chr5 | 141430772 | 141430838 | hg38 |
| chr5 | 141431238 | 141431303 | hg38 |
| chr5 | 141431335 | 141431439 | hg38 |
| chr5 | 141431494 | 141431570 | hg38 |
| chr5 | 141432048 | 141432109 | hg38 |
| chr5 | 141432286 | 141432326 | hg38 |
| chr5 | 141432439 | 141432470 | hg38 |
| chr5 | 141432502 | 141432596 | hg38 |
| chr5 | 141432612 | 141432668 | hg38 |
| chr5 | 141432742 | 141432755 | hg38 |
| chr5 | 141432853 | 141432866 | hg38 |
| chr5 | 141432966 | 141432973 | hg38 |
| chr5 | 141485567 | 141485577 | hg38 |
| chr5 | 146339595 | 146339623 | hg38 |
| chr5 | 146339982 | 146340020 | hg38 |
| chr5 | 146340278 | 146340295 | hg38 |
| chr5 | 146340306 | 146340349 | hg38 |
| chr5 | 146340456 | 146340465 | hg38 |
| chr5 | 146342918 | 146342955 | hg38 |
| chr5 | 146343118 | 146343168 | hg38 |
| chr5 | 146345816 | 146346116 | hg38 |
| chr5 | 150527971 | 150527972 | hg38 |
| chr5 | 150946420 | 150946437 | hg38 |
| chr5 | 171307872 | 171307891 | hg38 |
| chr5 | 171308166 | 171308189 | hg38 |
| chr5 | 171308382 | 171308404 | hg38 |
| chr5 | 171308476 | 171308494 | hg38 |
| chr5 | 171308668 | 171308696 | hg38 |
| chr5 | 171313901 | 171313934 | hg38 |
| chr5 | 171316188 | 171316197 | hg38 |
| chr5 | 173228885 | 173228897 | hg38 |
| chr5 | 173228922 | 173228935 | hg38 |
| chr5 | 173228953 | 173228975 | hg38 |
| chr5 | 173232417 | 173232422 | hg38 |
| chr5 | 173232646 | 173232906 | hg38 |
| chr5 | 173233943 | 173234031 | hg38 |
| chr5 | 173236768 | 173236829 | hg38 |
| chr5 | 173244744 | 173244776 | hg38 |
| chr5 | 173245370 | 173245388 | hg38 |
| chr5 | 173245530 | 173245545 | hg38 |
| chr5 | 173245646 | 173245682 | hg38 |
| chr5 | 173245698 | 173245703 | hg38 |
| chr5 | 174731928 | 174731974 | hg38 |
| chr5 | 174731981 | 174732037 | hg38 |
| chr5 | 174732175 | 174732202 | hg38 |
| chr5 | 174732327 | 174732404 | hg38 |
| chr5 | 174732499 | 174732511 | hg38 |
| chr5 | 176680177 | 176680201 | hg38 |
| chr5 | 178577203 | 178577209 | hg38 |
| chr6 | 1377984 | 1378112 | hg38 |
| chr6 | 1378840 | 1378861 | hg38 |
| chr6 | 1383595 | 1383625 | hg38 |
| chr6 | 1383680 | 1384100 | hg38 |
| chr6 | 1385434 | 1385484 | hg38 |
| chr6 | 1388787 | 1388922 | hg38 |
| chr6 | 1393680 | 1394090 | hg38 |
| chr6 | 1619688 | 1620188 | hg38 |
| chr6 | 2959279 | 2959280 | hg38 |
| chr6 | 4079051 | 4079059 | hg38 |
| chr6 | 6002239 | 6002279 | hg38 |
| chr6 | 6003594 | 6004394 | hg38 |
| chr6 | 6004405 | 6004545 | hg38 |
| chr6 | 6004700 | 6004716 | hg38 |
| chr6 | 7601487 | 7601488 | hg38 |
| chr6 | 10381305 | 10381565 | hg38 |
| chr6 | 10381848 | 10381885 | hg38 |
| chr6 | 10384611 | 10384748 | hg38 |
| chr6 | 10385240 | 10385257 | hg38 |
| chr6 | 10385548 | 10385775 | hg38 |
| chr6 | 10385927 | 10386027 | hg38 |
| chr6 | 10389846 | 10389869 | hg38 |
| chr6 | 10389953 | 10390008 | hg38 |
| chr6 | 10390213 | 10390256 | hg38 |
| chr6 | 10390471 | 10390484 | hg38 |
| chr6 | 10390656 | 10390687 | hg38 |
| chr6 | 10390793 | 10390827 | hg38 |
| chr6 | 10391092 | 10391109 | hg38 |
| chr6 | 10421275 | 10421289 | hg38 |
| chr6 | 10421806 | 10421880 | hg38 |
| chr6 | 10422156 | 10422215 | hg38 |
| chr6 | 10881763 | 10881786 | hg38 |
| chr6 | 17280804 | 17280819 | hg38 |
| chr6 | 25779164 | 25779165 | hg38 |
| chr6 | 26240469 | 26240723 | hg38 |
| chr6 | 26613818 | 26614107 | hg38 |
| chr6 | 26614115 | 26614615 | hg38 |
| chr6 | 27260934 | 27260971 | hg38 |
| chr6 | 27495581 | 27495600 | hg38 |
| chr6 | 27680031 | 27680046 | hg38 |
| chr6 | 27680220 | 27680246 | hg38 |
| chr6 | 27680552 | 27680633 | hg38 |
| chr6 | 28208206 | 28208233 | hg38 |
| chr6 | 28814293 | 28814309 | hg38 |
| chr6 | 28814396 | 28814426 | hg38 |
| chr6 | 28814505 | 28814520 | hg38 |
| chr6 | 29375048 | 29375049 | hg38 |
| chr6 | 38715163 | 38715373 | hg38 |
| chr6 | 42104194 | 42104834 | hg38 |
| chr6 | 43645263 | 43645278 | hg38 |
| chr6 | 50823672 | 50823754 | hg38 |
| chr6 | 50825641 | 50825756 | hg38 |
| chr6 | 50842905 | 50842985 | hg38 |
| chr6 | 50842999 | 50843133 | hg38 |
| chr6 | 50843218 | 50843233 | hg38 |
| chr6 | 50845608 | 50845629 | hg38 |
| chr6 | 50845706 | 50845716 | hg38 |
| chr6 | 50845746 | 50845793 | hg38 |
| chr6 | 51746672 | 51746673 | hg38 |
| chr6 | 70283028 | 70283039 | hg38 |
| chr6 | 71588628 | 71588636 | hg38 |
| chr6 | 71588659 | 71588668 | hg38 |
| chr6 | 73314799 | 73314810 | hg38 |
| chr6 | 73314921 | 73314958 | hg38 |
| chr6 | 73315023 | 73315048 | hg38 |
| chr6 | 77462484 | 77462884 | hg38 |
| chr6 | 83707879 | 83707914 | hg38 |
| chr6 | 84767011 | 84767043 | hg38 |
| chr6 | 84767512 | 84767532 | hg38 |
| chr6 | 84772866 | 84772895 | hg38 |
| chr6 | 84772962 | 84772974 | hg38 |
| chr6 | 98829346 | 98829359 | hg38 |
| chr6 | 98847890 | 98847905 | hg38 |
| chr6 | 99613167 | 99613179 | hg38 |
| chr6 | 100447326 | 100447370 | hg38 |
| chr6 | 100447713 | 100447763 | hg38 |
| chr6 | 100447898 | 100447992 | hg38 |
| chr6 | 100448106 | 100448164 | hg38 |
| chr6 | 100449637 | 100449649 | hg38 |
| chr6 | 100455778 | 100455816 | hg38 |
| chr6 | 100457569 | 100457607 | hg38 |
| chr6 | 100457721 | 100457818 | hg38 |
| chr6 | 100458231 | 100458260 | hg38 |
| chr6 | 100458273 | 100458343 | hg38 |
| chr6 | 100458344 | 100458369 | hg38 |
| chr6 | 100458399 | 100458429 | hg38 |
| chr6 | 100458646 | 100458672 | hg38 |
| chr6 | 100458745 | 100458760 | hg38 |
| chr6 | 100464210 | 100464230 | hg38 |
| chr6 | 100465384 | 100465398 | hg38 |
| chr6 | 100467838 | 100467892 | hg38 |
| chr6 | 100467976 | 100467985 | hg38 |
| chr6 | 100469055 | 100469095 | hg38 |
| chr6 | 101398990 | 101399014 | hg38 |
| chr6 | 101399822 | 101399837 | hg38 |
| chr6 | 105981836 | 105981873 | hg38 |
| chr6 | 105981962 | 105982071 | hg38 |
| chr6 | 105986149 | 105986161 | hg38 |
| chr6 | 105986170 | 105986192 | hg38 |
| chr6 | 105986194 | 105986257 | hg38 |
| chr6 | 105992997 | 105993073 | hg38 |
| chr6 | 108167163 | 108167199 | hg38 |
| chr6 | 108167390 | 108167440 | hg38 |
| chr6 | 108167675 | 108167712 | hg38 |
| chr6 | 108168070 | 108168077 | hg38 |
| chr6 | 108168302 | 108168372 | hg38 |
| chr6 | 108168452 | 108168543 | hg38 |
| chr6 | 108168657 | 108168797 | hg38 |
| chr6 | 108169053 | 108169114 | hg38 |
| chr6 | 108169251 | 108169380 | hg38 |
| chr6 | 108169657 | 108169791 | hg38 |
| chr6 | 108169846 | 108169923 | hg38 |
| chr6 | 108170748 | 108170799 | hg38 |
| chr6 | 108171063 | 108171116 | hg38 |
| chr6 | 108171130 | 108171203 | hg38 |
| chr6 | 108171460 | 108171526 | hg38 |
| chr6 | 108174460 | 108174494 | hg38 |
| chr6 | 108174495 | 108174527 | hg38 |
| chr6 | 108174661 | 108174730 | hg38 |
| chr6 | 108174810 | 108174885 | hg38 |
| chr6 | 133240511 | 133240582 | hg38 |
| chr6 | 133889643 | 133889663 | hg38 |
| chr6 | 133889911 | 133889920 | hg38 |
| chr6 | 133892975 | 133893039 | hg38 |
| chr6 | 136923127 | 136923447 | hg38 |
| chr6 | 137488493 | 137488513 | hg38 |
| chr6 | 137488844 | 137488897 | hg38 |
| chr6 | 137489267 | 137489357 | hg38 |
| chr6 | 137489575 | 137489729 | hg38 |
| chr6 | 137489934 | 137489974 | hg38 |
| chr6 | 137489988 | 137490106 | hg38 |
| chr6 | 137492890 | 137493017 | hg38 |
| chr6 | 137493631 | 137493658 | hg38 |
| chr6 | 137493820 | 137493852 | hg38 |
| chr6 | 137493893 | 137493900 | hg38 |
| chr6 | 137496003 | 137496028 | hg38 |
| chr6 | 148440320 | 148440321 | hg38 |
| chr6 | 149964432 | 149964535 | hg38 |
| chr6 | 149964806 | 149965379 | hg38 |
| chr6 | 166165610 | 166165676 | hg38 |
| chr6 | 166169896 | 166169928 | hg38 |
| chr6 | 168442382 | 168442399 | hg38 |
| chr7 | 1230542 | 1230550 | hg38 |
| chr7 | 1230576 | 1230604 | hg38 |
| chr7 | 1230609 | 1230626 | hg38 |
| chr7 | 1230979 | 1231003 | hg38 |
| chr7 | 1604008 | 1604021 | hg38 |
| chr7 | 5332649 | 5333086 | hg38 |
| chr7 | 6663983 | 6663998 | hg38 |
| chr7 | 8441175 | 8441207 | hg38 |
| chr7 | 8442345 | 8442386 | hg38 |
| chr7 | 8442390 | 8442426 | hg38 |
| chr7 | 8442602 | 8442628 | hg38 |
| chr7 | 8442730 | 8442793 | hg38 |
| chr7 | 8442908 | 8442943 | hg38 |
| chr7 | 12111594 | 12111924 | hg38 |
| chr7 | 19144392 | 19144405 | hg38 |
| chr7 | 19145035 | 19145048 | hg38 |
| chr7 | 19145348 | 19145369 | hg38 |
| chr7 | 20784376 | 20784420 | hg38 |
| chr7 | 20784427 | 20784437 | hg38 |
| chr7 | 20784766 | 20784790 | hg38 |
| chr7 | 24283939 | 24284389 | hg38 |
| chr7 | 24284973 | 24285003 | hg38 |
| chr7 | 25859052 | 25859069 | hg38 |
| chr7 | 27144990 | 27145039 | hg38 |
| chr7 | 27145048 | 27145057 | hg38 |
| chr7 | 27145118 | 27145143 | hg38 |
| chr7 | 27156830 | 27156871 | hg38 |
| chr7 | 27157005 | 27157133 | hg38 |
| chr7 | 27164362 | 27166272 | hg38 |
| chr7 | 27166406 | 27166460 | hg38 |
| chr7 | 27168569 | 27168583 | hg38 |
| chr7 | 27213546 | 27213569 | hg38 |
| chr7 | 27220514 | 27220540 | hg38 |
| chr7 | 27225385 | 27225396 | hg38 |
| chr7 | 27239526 | 27239796 | hg38 |
| chr7 | 27239882 | 27239967 | hg38 |
| chr7 | 27239968 | 27240099 | hg38 |
| chr7 | 27242496 | 27242526 | hg38 |
| chr7 | 27242868 | 27242875 | hg38 |
| chr7 | 27243598 | 27243685 | hg38 |
| chr7 | 27250186 | 27250234 | hg38 |
| chr7 | 27251600 | 27251638 | hg38 |
| chr7 | 27251646 | 27251670 | hg38 |
| chr7 | 27251680 | 27251696 | hg38 |
| chr7 | 27251861 | 27251868 | hg38 |
| chr7 | 27252364 | 27252373 | hg38 |
| chr7 | 27253177 | 27253197 | hg38 |
| chr7 | 32761909 | 32762023 | hg38 |
| chr7 | 32762158 | 32762208 | hg38 |
| chr7 | 32763047 | 32763071 | hg38 |
| chr7 | 32763082 | 32763182 | hg38 |
| chr7 | 35254851 | 35254876 | hg38 |
| chr7 | 35254892 | 35254912 | hg38 |
| chr7 | 35257728 | 35257750 | hg38 |
| chr7 | 35257789 | 35257794 | hg38 |
| chr7 | 35258090 | 35258098 | hg38 |
| chr7 | 35258407 | 35258418 | hg38 |
| chr7 | 35259025 | 35259058 | hg38 |
| chr7 | 35261540 | 35261578 | hg38 |
| chr7 | 35261856 | 35261864 | hg38 |
| chr7 | 35261948 | 35261981 | hg38 |
| chr7 | 42227917 | 42228337 | hg38 |
| chr7 | 64889046 | 64889536 | hg38 |
| chr7 | 71132933 | 71132938 | hg38 |
| chr7 | 77794257 | 77794258 | hg38 |
| chr7 | 93105138 | 93105139 | hg38 |
| chr7 | 97002119 | 97002183 | hg38 |
| chr7 | 97002368 | 97002396 | hg38 |
| chr7 | 97002428 | 97002445 | hg38 |
| chr7 | 97003502 | 97003532 | hg38 |
| chr7 | 97007284 | 97007305 | hg38 |
| chr7 | 97007352 | 97007421 | hg38 |
| chr7 | 97007434 | 97007469 | hg38 |
| chr7 | 97018121 | 97018126 | hg38 |
| chr7 | 97020940 | 97020956 | hg38 |
| chr7 | 97021111 | 97021127 | hg38 |
| chr7 | 97021131 | 97021181 | hg38 |
| chr7 | 97021191 | 97021206 | hg38 |
| chr7 | 97021381 | 97021406 | hg38 |
| chr7 | 97021438 | 97021449 | hg38 |
| chr7 | 97021469 | 97021481 | hg38 |
| chr7 | 97021936 | 97021968 | hg38 |
| chr7 | 97022126 | 97022174 | hg38 |
| chr7 | 97731929 | 97731941 | hg38 |
| chr7 | 97731945 | 97731968 | hg38 |
| chr7 | 97732082 | 97732093 | hg38 |
| chr7 | 97732122 | 97732136 | hg38 |
| chr7 | 97732142 | 97732151 | hg38 |
| chr7 | 97732189 | 97732208 | hg38 |
| chr7 | 100750761 | 100750762 | hg38 |
| chr7 | 108571909 | 108571910 | hg38 |
| chr7 | 114082986 | 114083044 | hg38 |
| chr7 | 122299845 | 122300024 | hg38 |
| chr7 | 122300027 | 122300160 | hg38 |
| chr7 | 122300162 | 122300193 | hg38 |
| chr7 | 122300378 | 122300408 | hg38 |
| chr7 | 122306771 | 122306891 | hg38 |
| chr7 | 122306951 | 122307038 | hg38 |
| chr7 | 122307044 | 122307071 | hg38 |
| chr7 | 122307369 | 122307427 | hg38 |
| chr7 | 122311923 | 122311948 | hg38 |
| chr7 | 122316836 | 122316872 | hg38 |
| chr7 | 122316874 | 122316888 | hg38 |
| chr7 | 122316897 | 122316916 | hg38 |
| chr7 | 128240899 | 128240993 | hg38 |
| chr7 | 128241277 | 128241320 | hg38 |
| chr7 | 128351542 | 128351616 | hg38 |
| chr7 | 128351663 | 128351688 | hg38 |
| chr7 | 128351714 | 128351743 | hg38 |
| chr7 | 128915807 | 128915836 | hg38 |
| chr7 | 129778570 | 129778575 | hg38 |
| chr7 | 129782834 | 129782869 | hg38 |
| chr7 | 129783007 | 129783131 | hg38 |
| chr7 | 129783191 | 129783196 | hg38 |
| chr7 | 129783205 | 129783223 | hg38 |
| chr7 | 129783261 | 129783340 | hg38 |
| chr7 | 129783378 | 129783402 | hg38 |
| chr7 | 129784693 | 129784832 | hg38 |
| chr7 | 129784926 | 129785186 | hg38 |
| chr7 | 130733316 | 130733338 | hg38 |
| chr7 | 131057307 | 131057308 | hg38 |
| chr7 | 137847472 | 137847505 | hg38 |
| chr7 | 137847613 | 137847636 | hg38 |
| chr7 | 142257684 | 142257685 | hg38 |
| chr7 | 149692578 | 149692623 | hg38 |
| chr7 | 149692635 | 149692663 | hg38 |
| chr7 | 149692675 | 149692722 | hg38 |
| chr7 | 149692754 | 149692811 | hg38 |
| chr7 | 152924835 | 152924866 | hg38 |
| chr7 | 153886949 | 153886962 | hg38 |
| chr7 | 153887311 | 153887321 | hg38 |
| chr7 | 155371736 | 155371778 | hg38 |
| chr7 | 155372446 | 155372492 | hg38 |
| chr7 | 155465558 | 155465602 | hg38 |
| chr7 | 155465695 | 155465745 | hg38 |
| chr7 | 155467634 | 155467657 | hg38 |
| chr7 | 155509962 | 155509998 | hg38 |
| chr7 | 155510425 | 155510446 | hg38 |
| chr7 | 155807889 | 155807991 | hg38 |
| chr7 | 156617076 | 156617157 | hg38 |
| chr7 | 157002820 | 157002854 | hg38 |
| chr7 | 157004410 | 157004428 | hg38 |
| chr7 | 157005141 | 157005391 | hg38 |
| chr7 | 157005491 | 157005801 | hg38 |
| chr7 | 157006142 | 157006190 | hg38 |
| chr7 | 157006371 | 157006402 | hg38 |
| chr7 | 157006597 | 157006604 | hg38 |
| chr7 | 157021798 | 157021899 | hg38 |
| chr7 | 157683837 | 157683842 | hg38 |
| chr7 | 157689293 | 157689310 | hg38 |
| chr7 | 157693409 | 157693421 | hg38 |
| chr7 | 159143846 | 159143859 | hg38 |
| chr7 | 159145712 | 159145757 | hg38 |
| chr8 | 9898952 | 9898963 | hg38 |
| chr8 | 10731795 | 10731800 | hg38 |
| chr8 | 10731998 | 10732045 | hg38 |
| chr8 | 10732079 | 10732118 | hg38 |
| chr8 | 10732149 | 10732230 | hg38 |
| chr8 | 11680129 | 11680161 | hg38 |
| chr8 | 11680304 | 11680328 | hg38 |
| chr8 | 11680487 | 11680554 | hg38 |
| chr8 | 11680711 | 11680734 | hg38 |
| chr8 | 21976454 | 21976455 | hg38 |
| chr8 | 22005040 | 22005041 | hg38 |
| chr8 | 23706462 | 23706812 | hg38 |
| chr8 | 23709885 | 23709896 | hg38 |
| chr8 | 24914234 | 24914259 | hg38 |
| chr8 | 25302369 | 25302370 | hg38 |
| chr8 | 35235961 | 35235986 | hg38 |
| chr8 | 41567300 | 41567378 | hg38 |
| chr8 | 41567663 | 41567717 | hg38 |
| chr8 | 48379899 | 48379927 | hg38 |
| chr8 | 49909813 | 49909822 | hg38 |
| chr8 | 52938591 | 52938601 | hg38 |
| chr8 | 53876734 | 53876775 | hg38 |
| chr8 | 53877069 | 53877153 | hg38 |
| chr8 | 53879286 | 53879294 | hg38 |
| chr8 | 53879298 | 53879339 | hg38 |
| chr8 | 54458480 | 54458502 | hg38 |
| chr8 | 54459487 | 54459495 | hg38 |
| chr8 | 54459644 | 54459676 | hg38 |
| chr8 | 54466620 | 54466630 | hg38 |
| chr8 | 54466766 | 54466780 | hg38 |
| chr8 | 54466888 | 54466899 | hg38 |
| chr8 | 54466909 | 54466921 | hg38 |
| chr8 | 54467085 | 54467098 | hg38 |
| chr8 | 54467263 | 54467273 | hg38 |
| chr8 | 54467559 | 54467586 | hg38 |
| chr8 | 56113625 | 56113632 | hg38 |
| chr8 | 56445571 | 56445590 | hg38 |
| chr8 | 56445708 | 56445726 | hg38 |
| chr8 | 56445727 | 56445805 | hg38 |
| chr8 | 56445863 | 56445887 | hg38 |
| chr8 | 64368939 | 64368947 | hg38 |
| chr8 | 64369449 | 64369461 | hg38 |
| chr8 | 64369466 | 64369886 | hg38 |
| chr8 | 64371082 | 64371125 | hg38 |
| chr8 | 64373522 | 64373541 | hg38 |
| chr8 | 64377438 | 64377445 | hg38 |
| chr8 | 64378171 | 64378189 | hg38 |
| chr8 | 64378192 | 64378224 | hg38 |
| chr8 | 64380800 | 64380806 | hg38 |
| chr8 | 64586035 | 64586050 | hg38 |
| chr8 | 64586055 | 64586066 | hg38 |
| chr8 | 64587218 | 64587245 | hg38 |
| chr8 | 64587348 | 64587370 | hg38 |
| chr8 | 66961187 | 66961206 | hg38 |
| chr8 | 70034684 | 70034710 | hg38 |
| chr8 | 70034718 | 70034736 | hg38 |
| chr8 | 70034906 | 70034984 | hg38 |
| chr8 | 70069528 | 70069834 | hg38 |
| chr8 | 70069847 | 70069901 | hg38 |
| chr8 | 70070178 | 70070210 | hg38 |
| chr8 | 70070344 | 70070387 | hg38 |
| chr8 | 70070432 | 70070440 | hg38 |
| chr8 | 70070584 | 70070608 | hg38 |
| chr8 | 70071171 | 70071189 | hg38 |
| chr8 | 70071474 | 70071507 | hg38 |
| chr8 | 70071519 | 70071541 | hg38 |
| chr8 | 70071652 | 70071689 | hg38 |
| chr8 | 70071753 | 70071776 | hg38 |
| chr8 | 70071780 | 70071791 | hg38 |
| chr8 | 70071954 | 70071967 | hg38 |
| chr8 | 70072008 | 70072325 | hg38 |
| chr8 | 70072369 | 70072424 | hg38 |
| chr8 | 70072481 | 70072509 | hg38 |
| chr8 | 70072543 | 70072607 | hg38 |
| chr8 | 70072682 | 70072746 | hg38 |
| chr8 | 71556335 | 71556351 | hg38 |
| chr8 | 71556363 | 71556372 | hg38 |
| chr8 | 71556382 | 71556393 | hg38 |
| chr8 | 71556394 | 71556403 | hg38 |
| chr8 | 71556695 | 71556895 | hg38 |
| chr8 | 71556968 | 71557025 | hg38 |
| chr8 | 71557144 | 71557188 | hg38 |
| chr8 | 71557278 | 71557289 | hg38 |
| chr8 | 71557972 | 71557987 | hg38 |
| chr8 | 71558289 | 71558297 | hg38 |
| chr8 | 71842234 | 71842259 | hg38 |
| chr8 | 71842266 | 71842288 | hg38 |
| chr8 | 71842291 | 71842313 | hg38 |
| chr8 | 71844410 | 71844415 | hg38 |
| chr8 | 72538296 | 72538311 | hg38 |
| chr8 | 75404592 | 75404600 | hg38 |
| chr8 | 76683896 | 76683917 | hg38 |
| chr8 | 84183780 | 84183800 | hg38 |
| chr8 | 84183802 | 84183813 | hg38 |
| chr8 | 84184527 | 84184540 | hg38 |
| chr8 | 84184884 | 84184916 | hg38 |
| chr8 | 85438049 | 85438089 | hg38 |
| chr8 | 85438134 | 85438162 | hg38 |
| chr8 | 85438339 | 85438383 | hg38 |
| chr8 | 86069497 | 86069510 | hg38 |
| chr8 | 86069514 | 86069536 | hg38 |
| chr8 | 86069538 | 86069627 | hg38 |
| chr8 | 86069633 | 86069687 | hg38 |
| chr8 | 94529074 | 94529075 | hg38 |
| chr8 | 96144536 | 96144599 | hg38 |
| chr8 | 96144626 | 96144632 | hg38 |
| chr8 | 96144670 | 96144695 | hg38 |
| chr8 | 96144716 | 96144753 | hg38 |
| chr8 | 96144809 | 96144824 | hg38 |
| chr8 | 96145065 | 96145802 | hg38 |
| chr8 | 96158001 | 96158026 | hg38 |
| chr8 | 96158150 | 96158177 | hg38 |
| chr8 | 96158193 | 96158764 | hg38 |
| chr8 | 96159389 | 96159419 | hg38 |
| chr8 | 96159781 | 96159788 | hg38 |
| chr8 | 98427699 | 98427708 | hg38 |
| chr8 | 98428003 | 98428028 | hg38 |
| chr8 | 98939180 | 98939261 | hg38 |
| chr8 | 98939596 | 98939645 | hg38 |
| chr8 | 98947129 | 98947223 | hg38 |
| chr8 | 98947227 | 98947308 | hg38 |
| chr8 | 98948807 | 98948895 | hg38 |
| chr8 | 98948947 | 98948985 | hg38 |
| chr8 | 98948993 | 98949031 | hg38 |
| chr8 | 98949050 | 98949149 | hg38 |
| chr8 | 98949170 | 98949195 | hg38 |
| chr8 | 98949395 | 98949434 | hg38 |
| chr8 | 98949536 | 98949581 | hg38 |
| chr8 | 98973633 | 98973775 | hg38 |
| chr8 | 98973796 | 98973805 | hg38 |
| chr8 | 98973839 | 98973891 | hg38 |
| chr8 | 98973893 | 98973921 | hg38 |
| chr8 | 98974016 | 98974047 | hg38 |
| chr8 | 98974093 | 98974141 | hg38 |
| chr8 | 98974225 | 98974249 | hg38 |
| chr8 | 98974250 | 98974255 | hg38 |
| chr8 | 98974307 | 98974535 | hg38 |
| chr8 | 98974623 | 98974657 | hg38 |
| chr8 | 100105793 | 100105861 | hg38 |
| chr8 | 100105959 | 100105976 | hg38 |
| chr8 | 100106018 | 100106030 | hg38 |
| chr8 | 100106053 | 100106060 | hg38 |
| chr8 | 100106231 | 100106238 | hg38 |
| chr8 | 103141525 | 103141563 | hg38 |
| chr8 | 104466558 | 104466594 | hg38 |
| chr8 | 104466717 | 104466747 | hg38 |
| chr8 | 104466755 | 104466765 | hg38 |
| chr8 | 104466915 | 104466935 | hg38 |
| chr8 | 104467055 | 104467066 | hg38 |
| chr8 | 108081649 | 108081732 | hg38 |
| chr8 | 113432127 | 113432202 | hg38 |
| chr8 | 113433620 | 113433635 | hg38 |
| chr8 | 124486306 | 124486307 | hg38 |
| chr8 | 131041882 | 131041903 | hg38 |
| chr8 | 131042224 | 131042270 | hg38 |
| chr8 | 131042481 | 131042490 | hg38 |
| chr8 | 133131869 | 133131870 | hg38 |
| chr8 | 138496862 | 138497212 | hg38 |
| chr8 | 138497217 | 138497229 | hg38 |
| chr8 | 141518311 | 141518316 | hg38 |
| chr8 | 141518337 | 141518347 | hg38 |
| chr8 | 141518525 | 141518540 | hg38 |
| chr8 | 142544265 | 142544282 | hg38 |
| chr8 | 143949719 | 143949749 | hg38 |
| chr8 | 143949760 | 143949801 | hg38 |
| chr8 | 144051404 | 144051443 | hg38 |
| chr8 | 144051940 | 144051946 | hg38 |
| chr8 | 144051968 | 144051992 | hg38 |
| chr9 | 116950 | 116972 | hg38 |
| chr9 | 117900 | 117910 | hg38 |
| chr9 | 117970 | 117998 | hg38 |
| chr9 | 118038 | 118060 | hg38 |
| chr9 | 118067 | 118098 | hg38 |
| chr9 | 842681 | 842693 | hg38 |
| chr9 | 843092 | 843109 | hg38 |
| chr9 | 843235 | 843250 | hg38 |
| chr9 | 970011 | 970028 | hg38 |
| chr9 | 970202 | 970231 | hg38 |
| chr9 | 970299 | 970434 | hg38 |
| chr9 | 970594 | 970606 | hg38 |
| chr9 | 970796 | 970823 | hg38 |
| chr9 | 971008 | 971030 | hg38 |
| chr9 | 971561 | 971587 | hg38 |
| chr9 | 974840 | 974982 | hg38 |
| chr9 | 1042545 | 1042553 | hg38 |
| chr9 | 1042573 | 1042584 | hg38 |
| chr9 | 1042594 | 1042608 | hg38 |
| chr9 | 1048965 | 1049008 | hg38 |
| chr9 | 1052560 | 1052574 | hg38 |
| chr9 | 21970986 | 21971017 | hg38 |
| chr9 | 21971139 | 21971187 | hg38 |
| chr9 | 23822807 | 23822820 | hg38 |
| chr9 | 23822833 | 23822898 | hg38 |
| chr9 | 37002501 | 37002518 | hg38 |
| chr9 | 74500018 | 74500064 | hg38 |
| chr9 | 77016508 | 77016582 | hg38 |
| chr9 | 77019970 | 77019992 | hg38 |
| chr9 | 77021907 | 77021978 | hg38 |
| chr9 | 77022093 | 77022210 | hg38 |
| chr9 | 77022757 | 77022944 | hg38 |
| chr9 | 77023010 | 77023025 | hg38 |
| chr9 | 77023042 | 77023061 | hg38 |
| chr9 | 77023062 | 77023139 | hg38 |
| chr9 | 77023211 | 77023241 | hg38 |
| chr9 | 86077421 | 86077422 | hg38 |
| chr9 | 89177583 | 89177649 | hg38 |
| chr9 | 93949253 | 93949271 | hg38 |
| chr9 | 93952765 | 93952902 | hg38 |
| chr9 | 93953637 | 93953679 | hg38 |
| chr9 | 93954705 | 93954769 | hg38 |
| chr9 | 95349750 | 95349828 | hg38 |
| chr9 | 96719647 | 96719682 | hg38 |
| chr9 | 96877340 | 96877394 | hg38 |
| chr9 | 97711383 | 97711400 | hg38 |
| chr9 | 97847673 | 97847855 | hg38 |
| chr9 | 97848315 | 97848518 | hg38 |
| chr9 | 97848850 | 97849037 | hg38 |
| chr9 | 97849146 | 97849182 | hg38 |
| chr9 | 97854983 | 97855004 | hg38 |
| chr9 | 97855021 | 97855070 | hg38 |
| chr9 | 97855081 | 97855102 | hg38 |
| chr9 | 97855168 | 97855180 | hg38 |
| chr9 | 97855409 | 97855431 | hg38 |
| chr9 | 97857456 | 97857704 | hg38 |
| chr9 | 99825722 | 99825744 | hg38 |
| chr9 | 119369768 | 119369777 | hg38 |
| chr9 | 120820914 | 120820915 | hg38 |
| chr9 | 124012457 | 124012827 | hg38 |
| chr9 | 124015166 | 124015189 | hg38 |
| chr9 | 124015305 | 124015354 | hg38 |
| chr9 | 124015480 | 124015498 | hg38 |
| chr9 | 124015535 | 124015548 | hg38 |
| chr9 | 124017407 | 124017495 | hg38 |
| chr9 | 124017572 | 124017605 | hg38 |
| chr9 | 124017819 | 124017852 | hg38 |
| chr9 | 126615153 | 126615186 | hg38 |
| chr9 | 126615284 | 126615328 | hg38 |
| chr9 | 126618748 | 126619198 | hg38 |
| chr9 | 126623997 | 126624011 | hg38 |
| chr9 | 126624075 | 126624102 | hg38 |
| chr9 | 126626899 | 126626931 | hg38 |
| chr9 | 132587382 | 132587411 | hg38 |
| chr9 | 132587420 | 132587458 | hg38 |
| chr10 | 7407709 | 7407744 | hg38 |
| chr10 | 7407876 | 7407911 | hg38 |
| chr10 | 8034354 | 8034367 | hg38 |
| chr10 | 8036339 | 8036363 | hg38 |
| chr10 | 8055680 | 8055707 | hg38 |
| chr10 | 16520215 | 16520455 | hg38 |
| chr10 | 22253283 | 22253373 | hg38 |
| chr10 | 22253401 | 22253421 | hg38 |
| chr10 | 22253440 | 22253453 | hg38 |
| chr10 | 22253457 | 22253497 | hg38 |
| chr10 | 22253519 | 22253581 | hg38 |
| chr10 | 22253722 | 22253764 | hg38 |
| chr10 | 22334473 | 22334495 | hg38 |
| chr10 | 22334501 | 22334512 | hg38 |
| chr10 | 22334716 | 22334741 | hg38 |
| chr10 | 22345178 | 22345207 | hg38 |
| chr10 | 22345216 | 22345247 | hg38 |
| chr10 | 22345297 | 22345311 | hg38 |
| chr10 | 22345316 | 22345328 | hg38 |
| chr10 | 22345757 | 22345781 | hg38 |
| chr10 | 22476231 | 22476260 | hg38 |
| chr10 | 22476282 | 22476307 | hg38 |
| chr10 | 22476312 | 22476354 | hg38 |
| chr10 | 22476511 | 22476517 | hg38 |
| chr10 | 22476530 | 22476685 | hg38 |
| chr10 | 22476783 | 22476839 | hg38 |
| chr10 | 22476847 | 22476889 | hg38 |
| chr10 | 22476997 | 22477039 | hg38 |
| chr10 | 22477217 | 22477275 | hg38 |
| chr10 | 22477442 | 22477462 | hg38 |
| chr10 | 22477499 | 22477574 | hg38 |
| chr10 | 23172402 | 23172412 | hg38 |
| chr10 | 23174086 | 23174095 | hg38 |
| chr10 | 23195197 | 23195211 | hg38 |
| chr10 | 23195230 | 23195243 | hg38 |
| chr10 | 26214862 | 26214969 | hg38 |
| chr10 | 26217869 | 26217880 | hg38 |
| chr10 | 26218276 | 26218295 | hg38 |
| chr10 | 27741447 | 27741489 | hg38 |
| chr10 | 27741542 | 27741553 | hg38 |
| chr10 | 27741699 | 27741819 | hg38 |
| chr10 | 27746790 | 27746809 | hg38 |
| chr10 | 45419240 | 45419306 | hg38 |
| chr10 | 45419327 | 45419387 | hg38 |
| chr10 | 49611080 | 49611098 | hg38 |
| chr10 | 49611256 | 49611317 | hg38 |
| chr10 | 49611464 | 49611491 | hg38 |
| chr10 | 49612026 | 49612039 | hg38 |
| chr10 | 49612186 | 49612228 | hg38 |
| chr10 | 70441008 | 70441032 | hg38 |
| chr10 | 70529945 | 70529946 | hg38 |
| chr10 | 97460128 | 97460129 | hg38 |
| chr10 | 99530644 | 99531394 | hg38 |
| chr10 | 99534076 | 99534106 | hg38 |
| chr10 | 100659427 | 100659438 | hg38 |
| chr10 | 100659452 | 100659467 | hg38 |
| chr10 | 100659586 | 100659608 | hg38 |
| chr10 | 100830394 | 100830638 | hg38 |
| chr10 | 101134343 | 101134603 | hg38 |
| chr10 | 101134930 | 101134940 | hg38 |
| chr10 | 101134943 | 101135513 | hg38 |
| chr10 | 101140126 | 101140132 | hg38 |
| chr10 | 101140482 | 101140572 | hg38 |
| chr10 | 101140652 | 101140696 | hg38 |
| chr10 | 101224147 | 101224167 | hg38 |
| chr10 | 101227571 | 101227587 | hg38 |
| chr10 | 101227650 | 101227657 | hg38 |
| chr10 | 101227739 | 101227761 | hg38 |
| chr10 | 101236367 | 101236463 | hg38 |
| chr10 | 101236500 | 101236570 | hg38 |
| chr10 | 101236728 | 101236818 | hg38 |
| chr10 | 101237084 | 101237186 | hg38 |
| chr10 | 101237316 | 101237339 | hg38 |
| chr10 | 101237345 | 101237473 | hg38 |
| chr10 | 101237480 | 101237603 | hg38 |
| chr10 | 101238023 | 101238061 | hg38 |
| chr10 | 101238115 | 101238237 | hg38 |
| chr10 | 101238537 | 101238681 | hg38 |
| chr10 | 101284240 | 101284322 | hg38 |
| chr10 | 101284333 | 101284567 | hg38 |
| chr10 | 101773501 | 101773530 | hg38 |
| chr10 | 102240689 | 102240873 | hg38 |
| chr10 | 102240921 | 102240952 | hg38 |
| chr10 | 109456869 | 109456889 | hg38 |
| chr10 | 116860948 | 116860949 | hg38 |
| chr10 | 117131682 | 117131756 | hg38 |
| chr10 | 117132672 | 117132703 | hg38 |
| chr10 | 117140104 | 117140143 | hg38 |
| chr10 | 117140163 | 117140179 | hg38 |
| chr10 | 117140739 | 117140768 | hg38 |
| chr10 | 117551391 | 117551409 | hg38 |
| chr10 | 117551801 | 117551820 | hg38 |
| chr10 | 117735109 | 117735149 | hg38 |
| chr10 | 118594446 | 118594488 | hg38 |
| chr10 | 118594662 | 118594673 | hg38 |
| chr10 | 118594739 | 118594793 | hg38 |
| chr10 | 118594869 | 118594901 | hg38 |
| chr10 | 118594985 | 118594990 | hg38 |
| chr10 | 118596222 | 118596232 | hg38 |
| chr10 | 120949009 | 120949101 | hg38 |
| chr10 | 120949124 | 120949268 | hg38 |
| chr10 | 122163215 | 122164055 | hg38 |
| chr10 | 122164527 | 122164571 | hg38 |
| chr10 | 123142657 | 123142693 | hg38 |
| chr10 | 123150701 | 123150737 | hg38 |
| chr10 | 127736426 | 127736656 | hg38 |
| chr10 | 132784160 | 132784230 | hg38 |
| chr10 | 132784344 | 132784398 | hg38 |
| chr10 | 132784783 | 132784801 | hg38 |
| chr10 | 132784942 | 132784959 | hg38 |
| chr10 | 132784967 | 132784995 | hg38 |
| chr11 | 627511 | 627561 | hg38 |
| chr11 | 1443460 | 1443481 | hg38 |
| chr11 | 4082294 | 4082295 | hg38 |
| chr11 | 4653345 | 4653346 | hg38 |
| chr11 | 11955081 | 11955082 | hg38 |
| chr11 | 16610962 | 16610979 | hg38 |
| chr11 | 16611022 | 16611090 | hg38 |
| chr11 | 16611149 | 16611174 | hg38 |
| chr11 | 17475964 | 17475976 | hg38 |
| chr11 | 17720131 | 17720138 | hg38 |
| chr11 | 20159039 | 20159062 | hg38 |
| chr11 | 20596372 | 20596392 | hg38 |
| chr11 | 22341054 | 22341094 | hg38 |
| chr11 | 23988984 | 23988985 | hg38 |
| chr11 | 31798324 | 31798343 | hg38 |
| chr11 | 31804196 | 31804758 | hg38 |
| chr11 | 31804786 | 31804793 | hg38 |
| chr11 | 31805048 | 31805099 | hg38 |
| chr11 | 31805100 | 31805116 | hg38 |
| chr11 | 31805152 | 31805161 | hg38 |
| chr11 | 31805198 | 31805213 | hg38 |
| chr11 | 31805246 | 31805287 | hg38 |
| chr11 | 31805536 | 31805578 | hg38 |
| chr11 | 31805817 | 31805907 | hg38 |
| chr11 | 31805970 | 31806091 | hg38 |
| chr11 | 31806357 | 31806444 | hg38 |
| chr11 | 31819982 | 31820043 | hg38 |
| chr11 | 31820100 | 31820174 | hg38 |
| chr11 | 31820211 | 31820222 | hg38 |
| chr11 | 31820392 | 31820410 | hg38 |
| chr11 | 31820438 | 31820569 | hg38 |
| chr11 | 31820615 | 31820640 | hg38 |
| chr11 | 31820798 | 31820898 | hg38 |
| chr11 | 31825314 | 31825327 | hg38 |
| chr11 | 31825592 | 31825627 | hg38 |
| chr11 | 32333254 | 32333844 | hg38 |
| chr11 | 32426905 | 32427495 | hg38 |
| chr11 | 32429018 | 32429046 | hg38 |
| chr11 | 32433879 | 32433910 | hg38 |
| chr11 | 32438289 | 32438330 | hg38 |
| chr11 | 43583203 | 43583215 | hg38 |
| chr11 | 44303372 | 44303409 | hg38 |
| chr11 | 44304710 | 44304715 | hg38 |
| chr11 | 44304895 | 44304915 | hg38 |
| chr11 | 44304935 | 44304959 | hg38 |
| chr11 | 46366355 | 46366985 | hg38 |
| chr11 | 60951540 | 60951577 | hg38 |
| chr11 | 62138902 | 62138903 | hg38 |
| chr11 | 62925901 | 62926511 | hg38 |
| chr11 | 62927082 | 62927116 | hg38 |
| chr11 | 65047763 | 65047771 | hg38 |
| chr11 | 69986099 | 69986106 | hg38 |
| chr11 | 70365399 | 70365418 | hg38 |
| chr11 | 72241187 | 72241210 | hg38 |
| chr11 | 72241232 | 72241245 | hg38 |
| chr11 | 106042257 | 106042258 | hg38 |
| chr11 | 117465006 | 117465007 | hg38 |
| chr11 | 119076924 | 119076925 | hg38 |
| chr11 | 124896232 | 124896233 | hg38 |
| chr12 | 884764 | 884765 | hg38 |
| chr12 | 4164734 | 4164752 | hg38 |
| chr12 | 6555259 | 6556170 | hg38 |
| chr12 | 7872820 | 7872856 | hg38 |
| chr12 | 7872864 | 7872875 | hg38 |
| chr12 | 7872880 | 7872890 | hg38 |
| chr12 | 7873050 | 7873071 | hg38 |
| chr12 | 7873089 | 7873108 | hg38 |
| chr12 | 7873208 | 7873215 | hg38 |
| chr12 | 13563704 | 13563705 | hg38 |
| chr12 | 21941059 | 21941076 | hg38 |
| chr12 | 21941202 | 21941234 | hg38 |
| chr12 | 21941240 | 21941249 | hg38 |
| chr12 | 21941765 | 21941779 | hg38 |
| chr12 | 21941818 | 21941832 | hg38 |
| chr12 | 26911299 | 26911300 | hg38 |
| chr12 | 45049999 | 45050030 | hg38 |
| chr12 | 48836252 | 48836253 | hg38 |
| chr12 | 48997195 | 48997269 | hg38 |
| chr12 | 49296194 | 49296211 | hg38 |
| chr12 | 49342004 | 49342036 | hg38 |
| chr12 | 52258325 | 52258338 | hg38 |
| chr12 | 52258373 | 52258386 | hg38 |
| chr12 | 52258403 | 52258774 | hg38 |
| chr12 | 53738368 | 53738399 | hg38 |
| chr12 | 53738522 | 53738532 | hg38 |
| chr12 | 53927550 | 53927633 | hg38 |
| chr12 | 53927734 | 53927743 | hg38 |
| chr12 | 53944898 | 53945041 | hg38 |
| chr12 | 53945153 | 53945229 | hg38 |
| chr12 | 53945294 | 53945334 | hg38 |
| chr12 | 53945376 | 53945518 | hg38 |
| chr12 | 53960757 | 53960778 | hg38 |
| chr12 | 53960817 | 53960824 | hg38 |
| chr12 | 53966639 | 53966693 | hg38 |
| chr12 | 53966820 | 53966832 | hg38 |
| chr12 | 53966840 | 53966858 | hg38 |
| chr12 | 54029665 | 54029676 | hg38 |
| chr12 | 54029756 | 54029763 | hg38 |
| chr12 | 54030852 | 54030890 | hg38 |
| chr12 | 54031180 | 54031259 | hg38 |
| chr12 | 54047346 | 54047352 | hg38 |
| chr12 | 54047859 | 54047881 | hg38 |
| chr12 | 54053160 | 54053255 | hg38 |
| chr12 | 54107247 | 54107282 | hg38 |
| chr12 | 57224810 | 57224849 | hg38 |
| chr12 | 57224916 | 57225236 | hg38 |
| chr12 | 57256057 | 57256058 | hg38 |
| chr12 | 57627511 | 57627961 | hg38 |
| chr12 | 57628048 | 57628060 | hg38 |
| chr12 | 57628170 | 57628177 | hg38 |
| chr12 | 57628193 | 57628212 | hg38 |
| chr12 | 57631883 | 57631906 | hg38 |
| chr12 | 62191684 | 62191692 | hg38 |
| chr12 | 63149862 | 63149975 | hg38 |
| chr12 | 63150020 | 63150079 | hg38 |
| chr12 | 63150098 | 63150197 | hg38 |
| chr12 | 63150288 | 63150322 | hg38 |
| chr12 | 63150323 | 63150344 | hg38 |
| chr12 | 63150381 | 63150396 | hg38 |
| chr12 | 63150423 | 63150438 | hg38 |
| chr12 | 63150461 | 63150491 | hg38 |
| chr12 | 63150597 | 63150642 | hg38 |
| chr12 | 64824982 | 64825010 | hg38 |
| chr12 | 69574082 | 69574083 | hg38 |
| chr12 | 75207440 | 75207447 | hg38 |
| chr12 | 75207489 | 75207506 | hg38 |
| chr12 | 75207554 | 75207624 | hg38 |
| chr12 | 75207660 | 75207672 | hg38 |
| chr12 | 75207685 | 75207705 | hg38 |
| chr12 | 75207738 | 75207759 | hg38 |
| chr12 | 75207798 | 75207814 | hg38 |
| chr12 | 75207816 | 75207835 | hg38 |
| chr12 | 75209288 | 75209312 | hg38 |
| chr12 | 80708365 | 80708695 | hg38 |
| chr12 | 80708723 | 80708735 | hg38 |
| chr12 | 80708737 | 80708767 | hg38 |
| chr12 | 80708772 | 80708915 | hg38 |
| chr12 | 85278514 | 85278548 | hg38 |
| chr12 | 85278549 | 85278626 | hg38 |
| chr12 | 85278760 | 85278801 | hg38 |
| chr12 | 85279094 | 85279163 | hg38 |
| chr12 | 85279547 | 85279576 | hg38 |
| chr12 | 100716260 | 100716283 | hg38 |
| chr12 | 107775238 | 107775263 | hg38 |
| chr12 | 107775780 | 107775793 | hg38 |
| chr12 | 112575597 | 112575633 | hg38 |
| chr12 | 113478841 | 113478862 | hg38 |
| chr12 | 113480052 | 113480072 | hg38 |
| chr12 | 114404720 | 114404736 | hg38 |
| chr12 | 114408103 | 114408131 | hg38 |
| chr12 | 114408989 | 114409019 | hg38 |
| chr12 | 114409126 | 114409756 | hg38 |
| chr12 | 114409802 | 114409826 | hg38 |
| chr12 | 114448166 | 114448182 | hg38 |
| chr12 | 114697198 | 114697241 | hg38 |
| chr12 | 114697252 | 114697261 | hg38 |
| chr12 | 116508530 | 116508545 | hg38 |
| chr12 | 130163554 | 130163569 | hg38 |
| chr12 | 130163577 | 130163604 | hg38 |
| chr12 | 132569931 | 132571675 | hg38 |
| chr13 | 20301604 | 20301641 | hg38 |
| chr13 | 20301786 | 20301828 | hg38 |
| chr13 | 20946220 | 20946241 | hg38 |
| chr13 | 24746028 | 24746048 | hg38 |
| chr13 | 24746201 | 24746229 | hg38 |
| chr13 | 25469322 | 25469348 | hg38 |
| chr13 | 27050219 | 27050220 | hg38 |
| chr13 | 27924092 | 27924108 | hg38 |
| chr13 | 27924295 | 27924302 | hg38 |
| chr13 | 27924649 | 27924674 | hg38 |
| chr13 | 27924678 | 27924697 | hg38 |
| chr13 | 27970476 | 27970485 | hg38 |
| chr13 | 27970501 | 27970551 | hg38 |
| chr13 | 27970673 | 27970682 | hg38 |
| chr13 | 35471014 | 35471034 | hg38 |
| chr13 | 49221169 | 49221189 | hg38 |
| chr13 | 50843277 | 50843290 | hg38 |
| chr13 | 50843453 | 50843503 | hg38 |
| chr13 | 50843659 | 50843677 | hg38 |
| chr13 | 52847534 | 52847554 | hg38 |
| chr13 | 52847870 | 52847891 | hg38 |
| chr13 | 57629242 | 57629882 | hg38 |
| chr13 | 57629892 | 57630162 | hg38 |
| chr13 | 57633011 | 57633027 | hg38 |
| chr13 | 57633045 | 57633069 | hg38 |
| chr13 | 57633092 | 57633108 | hg38 |
| chr13 | 57633218 | 57633276 | hg38 |
| chr13 | 57633305 | 57633351 | hg38 |
| chr13 | 57633356 | 57633435 | hg38 |
| chr13 | 57633559 | 57633612 | hg38 |
| chr13 | 57633653 | 57633670 | hg38 |
| chr13 | 78596037 | 78596080 | hg38 |
| chr13 | 78596101 | 78596129 | hg38 |
| chr13 | 78596330 | 78596341 | hg38 |
| chr13 | 78596824 | 78596856 | hg38 |
| chr13 | 94701969 | 94702000 | hg38 |
| chr13 | 94702327 | 94702355 | hg38 |
| chr13 | 94702709 | 94702728 | hg38 |
| chr13 | 94702752 | 94702780 | hg38 |
| chr13 | 94702795 | 94702812 | hg38 |
| chr13 | 94703842 | 94703867 | hg38 |
| chr13 | 94703946 | 94703953 | hg38 |
| chr13 | 99978567 | 99978574 | hg38 |
| chr13 | 99987792 | 99987812 | hg38 |
| chr13 | 99987818 | 99987839 | hg38 |
| chr13 | 99989236 | 99989246 | hg38 |
| chr13 | 99991514 | 99991538 | hg38 |
| chr13 | 108495260 | 108496860 | hg38 |
| chr13 | 112056715 | 112056739 | hg38 |
| chr13 | 112057448 | 112057468 | hg38 |
| chr13 | 112057487 | 112057503 | hg38 |
| chr13 | 112057840 | 112058680 | hg38 |
| chr13 | 112066286 | 112067020 | hg38 |
| chr13 | 112068386 | 112068406 | hg38 |
| chr13 | 112069197 | 112069214 | hg38 |
| chr13 | 112069215 | 112069235 | hg38 |
| chr13 | 112069888 | 112069910 | hg38 |
| chr13 | 112103978 | 112103986 | hg38 |
| chr13 | 112104526 | 112104540 | hg38 |
| chr14 | 23575895 | 23575921 | hg38 |
| chr14 | 23575987 | 23576023 | hg38 |
| chr14 | 23576218 | 23576224 | hg38 |
| chr14 | 24334499 | 24334525 | hg38 |
| chr14 | 24334542 | 24335152 | hg38 |
| chr14 | 26204801 | 26204824 | hg38 |
| chr14 | 26204958 | 26204997 | hg38 |
| chr14 | 26205003 | 26205012 | hg38 |
| chr14 | 28765752 | 28765776 | hg38 |
| chr14 | 28765967 | 28765981 | hg38 |
| chr14 | 28766544 | 28766566 | hg38 |
| chr14 | 28767840 | 28767863 | hg38 |
| chr14 | 28767880 | 28767904 | hg38 |
| chr14 | 28768017 | 28768026 | hg38 |
| chr14 | 28768080 | 28768119 | hg38 |
| chr14 | 28768148 | 28768189 | hg38 |
| chr14 | 28768263 | 28768275 | hg38 |
| chr14 | 28768353 | 28768376 | hg38 |
| chr14 | 28768386 | 28768435 | hg38 |
| chr14 | 28785563 | 28785592 | hg38 |
| chr14 | 36504429 | 36504435 | hg38 |
| chr14 | 36505172 | 36505232 | hg38 |
| chr14 | 36516693 | 36516708 | hg38 |
| chr14 | 36521958 | 36521981 | hg38 |
| chr14 | 36522574 | 36522640 | hg38 |
| chr14 | 36523120 | 36523140 | hg38 |
| chr14 | 36524491 | 36524505 | hg38 |
| chr14 | 36654401 | 36654574 | hg38 |
| chr14 | 36654813 | 36654849 | hg38 |
| chr14 | 36655014 | 36655055 | hg38 |
| chr14 | 36655139 | 36655186 | hg38 |
| chr14 | 36656707 | 36656836 | hg38 |
| chr14 | 36657383 | 36657863 | hg38 |
| chr14 | 36658511 | 36658670 | hg38 |
| chr14 | 36658693 | 36658704 | hg38 |
| chr14 | 36658749 | 36658763 | hg38 |
| chr14 | 36660944 | 36661042 | hg38 |
| chr14 | 36666549 | 36666596 | hg38 |
| chr14 | 36666654 | 36666696 | hg38 |
| chr14 | 36666714 | 36666745 | hg38 |
| chr14 | 36667053 | 36667079 | hg38 |
| chr14 | 38209905 | 38209944 | hg38 |
| chr14 | 38209978 | 38210042 | hg38 |
| chr14 | 38210173 | 38210184 | hg38 |
| chr14 | 38210514 | 38210544 | hg38 |
| chr14 | 38210842 | 38210912 | hg38 |
| chr14 | 38211388 | 38211408 | hg38 |
| chr14 | 38211458 | 38211480 | hg38 |
| chr14 | 38255050 | 38255079 | hg38 |
| chr14 | 38255349 | 38256389 | hg38 |
| chr14 | 56794435 | 56794460 | hg38 |
| chr14 | 56794465 | 56794476 | hg38 |
| chr14 | 56794595 | 56794616 | hg38 |
| chr14 | 56795101 | 56795131 | hg38 |
| chr14 | 56795618 | 56795624 | hg38 |
| chr14 | 56798743 | 56798755 | hg38 |
| chr14 | 56798764 | 56798774 | hg38 |
| chr14 | 56799311 | 56799339 | hg38 |
| chr14 | 56804449 | 56804535 | hg38 |
| chr14 | 56807898 | 56807904 | hg38 |
| chr14 | 56807910 | 56807943 | hg38 |
| chr14 | 56808765 | 56808800 | hg38 |
| chr14 | 56808915 | 56808952 | hg38 |
| chr14 | 56808959 | 56808973 | hg38 |
| chr14 | 56809105 | 56809114 | hg38 |
| chr14 | 56809158 | 56809179 | hg38 |
| chr14 | 56809516 | 56809525 | hg38 |
| chr14 | 56809887 | 56809898 | hg38 |
| chr14 | 56809900 | 56809917 | hg38 |
| chr14 | 56809919 | 56809959 | hg38 |
| chr14 | 56810038 | 56810105 | hg38 |
| chr14 | 56810332 | 56810353 | hg38 |
| chr14 | 56811232 | 56811359 | hg38 |
| chr14 | 56812136 | 56812210 | hg38 |
| chr14 | 56812309 | 56812371 | hg38 |
| chr14 | 56817033 | 56817074 | hg38 |
| chr14 | 56817135 | 56817140 | hg38 |
| chr14 | 56817414 | 56817460 | hg38 |
| chr14 | 56817810 | 56817838 | hg38 |
| chr14 | 60486109 | 60486140 | hg38 |
| chr14 | 60486152 | 60486161 | hg38 |
| chr14 | 60507105 | 60507159 | hg38 |
| chr14 | 60507321 | 60507340 | hg38 |
| chr14 | 60508729 | 60508815 | hg38 |
| chr14 | 60509810 | 60509829 | hg38 |
| chr14 | 60510476 | 60510540 | hg38 |
| chr14 | 60510645 | 60510680 | hg38 |
| chr14 | 60510850 | 60510867 | hg38 |
| chr14 | 60511409 | 60511416 | hg38 |
| chr14 | 60511432 | 60511535 | hg38 |
| chr14 | 60515559 | 60515580 | hg38 |
| chr14 | 60515649 | 60515687 | hg38 |
| chr14 | 60515691 | 60515708 | hg38 |
| chr14 | 60515745 | 60515765 | hg38 |
| chr14 | 60515890 | 60515964 | hg38 |
| chr14 | 60637568 | 60637603 | hg38 |
| chr14 | 60637734 | 60637739 | hg38 |
| chr14 | 60637889 | 60637907 | hg38 |
| chr14 | 60637938 | 60638131 | hg38 |
| chr14 | 60642055 | 60642071 | hg38 |
| chr14 | 60642120 | 60642144 | hg38 |
| chr14 | 60642237 | 60642311 | hg38 |
| chr14 | 60642398 | 60642419 | hg38 |
| chr14 | 60642425 | 60642460 | hg38 |
| chr14 | 60642489 | 60642538 | hg38 |
| chr14 | 60642720 | 60642728 | hg38 |
| chr14 | 60642738 | 60642815 | hg38 |
| chr14 | 60643001 | 60643068 | hg38 |
| chr14 | 60643149 | 60643226 | hg38 |
| chr14 | 60643325 | 60643376 | hg38 |
| chr14 | 60655388 | 60655456 | hg38 |
| chr14 | 60655572 | 60655642 | hg38 |
| chr14 | 77459668 | 77459669 | hg38 |
| chr14 | 93787690 | 93787709 | hg38 |
| chr14 | 93787799 | 93787808 | hg38 |
| chr14 | 93787834 | 93787845 | hg38 |
| chr14 | 94771388 | 94771420 | hg38 |
| chr14 | 94771647 | 94771705 | hg38 |
| chr14 | 94773261 | 94773308 | hg38 |
| chr14 | 94773311 | 94773333 | hg38 |
| chr14 | 94774218 | 94774231 | hg38 |
| chr14 | 100726684 | 100727164 | hg38 |
| chr14 | 101456916 | 101456936 | hg38 |
| chr14 | 101457693 | 101457716 | hg38 |
| chr14 | 103188933 | 103188948 | hg38 |
| chr14 | 103274065 | 103274080 | hg38 |
| chr14 | 103274378 | 103274404 | hg38 |
| chr14 | 103274406 | 103274426 | hg38 |
| chr14 | 103274475 | 103274517 | hg38 |
| chr15 | 26967813 | 26967832 | hg38 |
| chr15 | 26968011 | 26968041 | hg38 |
| chr15 | 36879914 | 36879968 | hg38 |
| chr15 | 45116191 | 45116199 | hg38 |
| chr15 | 45116572 | 45116664 | hg38 |
| chr15 | 45135307 | 45135322 | hg38 |
| chr15 | 45135350 | 45135405 | hg38 |
| chr15 | 45135539 | 45135547 | hg38 |
| chr15 | 45135635 | 45135703 | hg38 |
| chr15 | 52783281 | 52783313 | hg38 |
| chr15 | 52805781 | 52805803 | hg38 |
| chr15 | 58064967 | 58065007 | hg38 |
| chr15 | 67826223 | 67826247 | hg38 |
| chr15 | 67826365 | 67826401 | hg38 |
| chr15 | 67826787 | 67826808 | hg38 |
| chr15 | 67826926 | 67826937 | hg38 |
| chr15 | 67829858 | 67829873 | hg38 |
| chr15 | 67829883 | 67829890 | hg38 |
| chr15 | 67829908 | 67829942 | hg38 |
| chr15 | 67830081 | 67830091 | hg38 |
| chr15 | 68331952 | 68331953 | hg38 |
| chr15 | 73249172 | 73249173 | hg38 |
| chr15 | 74134196 | 74134243 | hg38 |
| chr15 | 75358495 | 75358496 | hg38 |
| chr15 | 76338100 | 76338172 | hg38 |
| chr15 | 76338258 | 76338618 | hg38 |
| chr15 | 76339861 | 76339947 | hg38 |
| chr15 | 76340073 | 76340153 | hg38 |
| chr15 | 76340461 | 76340570 | hg38 |
| chr15 | 76340614 | 76340746 | hg38 |
| chr15 | 76342768 | 76342978 | hg38 |
| chr15 | 78497146 | 78497147 | hg38 |
| chr15 | 79433250 | 79433299 | hg38 |
| chr15 | 84924044 | 84924045 | hg38 |
| chr15 | 88606713 | 88606722 | hg38 |
| chr15 | 89370510 | 89370575 | hg38 |
| chr15 | 89378975 | 89379355 | hg38 |
| chr15 | 89406197 | 89406230 | hg38 |
| chr15 | 89406430 | 89406550 | hg38 |
| chr15 | 89408268 | 89408393 | hg38 |
| chr15 | 89408904 | 89409042 | hg38 |
| chr15 | 89409128 | 89409139 | hg38 |
| chr15 | 89409240 | 89409258 | hg38 |
| chr15 | 89409268 | 89409275 | hg38 |
| chr15 | 89409459 | 89409594 | hg38 |
| chr15 | 89409683 | 89409754 | hg38 |
| chr15 | 89409971 | 89410052 | hg38 |
| chr15 | 89814183 | 89814207 | hg38 |
| chr15 | 89814268 | 89814290 | hg38 |
| chr15 | 89814353 | 89814373 | hg38 |
| chr15 | 89814442 | 89814451 | hg38 |
| chr15 | 96360707 | 96360742 | hg38 |
| chr15 | 96365934 | 96365960 | hg38 |
| chr15 | 96366093 | 96366156 | hg38 |
| chr15 | 96366322 | 96366333 | hg38 |
| chr15 | 96367678 | 96367691 | hg38 |
| chr16 | 565047 | 565074 | hg38 |
| chr16 | 3170981 | 3171089 | hg38 |
| chr16 | 3171175 | 3171280 | hg38 |
| chr16 | 3171960 | 3172040 | hg38 |
| chr16 | 3184314 | 3184325 | hg38 |
| chr16 | 4805278 | 4805279 | hg38 |
| chr16 | 31569238 | 31569702 | hg38 |
| chr16 | 49277883 | 49277899 | hg38 |
| chr16 | 49278606 | 49278628 | hg38 |
| chr16 | 51149509 | 51149518 | hg38 |
| chr16 | 51153156 | 51153174 | hg38 |
| chr16 | 51154204 | 51154211 | hg38 |
| chr16 | 54288387 | 54288502 | hg38 |
| chr16 | 54289026 | 54289086 | hg38 |
| chr16 | 54289262 | 54289389 | hg38 |
| chr16 | 54289423 | 54289433 | hg38 |
| chr16 | 54289443 | 54289507 | hg38 |
| chr16 | 54289679 | 54289717 | hg38 |
| chr16 | 54290185 | 54290276 | hg38 |
| chr16 | 54290425 | 54290486 | hg38 |
| chr16 | 54290526 | 54290559 | hg38 |
| chr16 | 54290600 | 54290636 | hg38 |
| chr16 | 54291523 | 54291546 | hg38 |
| chr16 | 54291709 | 54291753 | hg38 |
| chr16 | 54936339 | 54936669 | hg38 |
| chr16 | 54937039 | 54937669 | hg38 |
| chr16 | 55329049 | 55329062 | hg38 |
| chr16 | 55330931 | 55330979 | hg38 |
| chr16 | 55331096 | 55331149 | hg38 |
| chr16 | 55331903 | 55331915 | hg38 |
| chr16 | 55332038 | 55332068 | hg38 |
| chr16 | 58197361 | 58197706 | hg38 |
| chr16 | 66578847 | 66578887 | hg38 |
| chr16 | 66578956 | 66579334 | hg38 |
| chr16 | 66579363 | 66579399 | hg38 |
| chr16 | 67163179 | 67163979 | hg38 |
| chr16 | 67165389 | 67165445 | hg38 |
| chr16 | 67165462 | 67165493 | hg38 |
| chr16 | 67165727 | 67165862 | hg38 |
| chr16 | 67165937 | 67165980 | hg38 |
| chr16 | 70481452 | 70481453 | hg38 |
| chr16 | 73062943 | 73063036 | hg38 |
| chr16 | 73063158 | 73063180 | hg38 |
| chr16 | 86504661 | 86504702 | hg38 |
| chr16 | 86511253 | 86511269 | hg38 |
| chr16 | 86511345 | 86511361 | hg38 |
| chr16 | 86511390 | 86511397 | hg38 |
| chr16 | 86513969 | 86513974 | hg38 |
| chr16 | 86578822 | 86579242 | hg38 |
| chr17 | 5096834 | 5097644 | hg38 |
| chr17 | 5097752 | 5097758 | hg38 |
| chr17 | 17264850 | 17264851 | hg38 |
| chr17 | 28711791 | 28711831 | hg38 |
| chr17 | 34156908 | 34157238 | hg38 |
| chr17 | 35449766 | 35449793 | hg38 |
| chr17 | 36641402 | 36641433 | hg38 |
| chr17 | 36939516 | 36939578 | hg38 |
| chr17 | 36940551 | 36940656 | hg38 |
| chr17 | 36940668 | 36940687 | hg38 |
| chr17 | 36940853 | 36940879 | hg38 |
| chr17 | 36942086 | 36942139 | hg38 |
| chr17 | 36942218 | 36942264 | hg38 |
| chr17 | 36942266 | 36942282 | hg38 |
| chr17 | 36942290 | 36942304 | hg38 |
| chr17 | 36942730 | 36942748 | hg38 |
| chr17 | 36942917 | 36942925 | hg38 |
| chr17 | 36943016 | 36943030 | hg38 |
| chr17 | 36943052 | 36943081 | hg38 |
| chr17 | 36943575 | 36943683 | hg38 |
| chr17 | 38561690 | 38561723 | hg38 |
| chr17 | 39164479 | 39165559 | hg38 |
| chr17 | 46819618 | 46819638 | hg38 |
| chr17 | 47831418 | 47831701 | hg38 |
| chr17 | 48541565 | 48542005 | hg38 |
| chr17 | 48548005 | 48548032 | hg38 |
| chr17 | 48592123 | 48592131 | hg38 |
| chr17 | 48592316 | 48592338 | hg38 |
| chr17 | 48592348 | 48592355 | hg38 |
| chr17 | 48633710 | 48633990 | hg38 |
| chr17 | 48718363 | 48718373 | hg38 |
| chr17 | 48718619 | 48718681 | hg38 |
| chr17 | 48718742 | 48718838 | hg38 |
| chr17 | 48718873 | 48719001 | hg38 |
| chr17 | 48719080 | 48719091 | hg38 |
| chr17 | 48719293 | 48719303 | hg38 |
| chr17 | 48719808 | 48719847 | hg38 |
| chr17 | 48720107 | 48720157 | hg38 |
| chr17 | 48722360 | 48722394 | hg38 |
| chr17 | 48722496 | 48722625 | hg38 |
| chr17 | 48726986 | 48727005 | hg38 |
| chr17 | 48734584 | 48734616 | hg38 |
| chr17 | 48747445 | 48747466 | hg38 |
| chr17 | 48747560 | 48747650 | hg38 |
| chr17 | 48747909 | 48748010 | hg38 |
| chr17 | 48749664 | 48749752 | hg38 |
| chr17 | 48750141 | 48750164 | hg38 |
| chr17 | 48750208 | 48750259 | hg38 |
| chr17 | 49470035 | 49470054 | hg38 |
| chr17 | 49964763 | 49964779 | hg38 |
| chr17 | 49964891 | 49964956 | hg38 |
| chr17 | 49965186 | 49965201 | hg38 |
| chr17 | 49965208 | 49965286 | hg38 |
| chr17 | 49972910 | 49972955 | hg38 |
| chr17 | 49973107 | 49973129 | hg38 |
| chr17 | 61395777 | 61395790 | hg38 |
| chr17 | 61395793 | 61395815 | hg38 |
| chr17 | 61396002 | 61396045 | hg38 |
| chr17 | 61398403 | 61398415 | hg38 |
| chr17 | 61401579 | 61401593 | hg38 |
| chr17 | 61401778 | 61401800 | hg38 |
| chr17 | 61405755 | 61405777 | hg38 |
| chr17 | 61405853 | 61405862 | hg38 |
| chr17 | 61408360 | 61408384 | hg38 |
| chr17 | 61451624 | 61451742 | hg38 |
| chr17 | 61451905 | 61451978 | hg38 |
| chr17 | 61452207 | 61452212 | hg38 |
| chr17 | 61452481 | 61452498 | hg38 |
| chr17 | 61452501 | 61452516 | hg38 |
| chr17 | 61452773 | 61452800 | hg38 |
| chr17 | 61452822 | 61452900 | hg38 |
| chr17 | 61454374 | 61454424 | hg38 |
| chr17 | 61454477 | 61454490 | hg38 |
| chr17 | 61454669 | 61454686 | hg38 |
| chr17 | 61454697 | 61454737 | hg38 |
| chr17 | 61454774 | 61454788 | hg38 |
| chr17 | 61454836 | 61454844 | hg38 |
| chr17 | 61454856 | 61454893 | hg38 |
| chr17 | 61454900 | 61454984 | hg38 |
| chr17 | 61455025 | 61455034 | hg38 |
| chr17 | 61455377 | 61455466 | hg38 |
| chr17 | 61456391 | 61456415 | hg38 |
| chr17 | 61456514 | 61456534 | hg38 |
| chr17 | 61456540 | 61456566 | hg38 |
| chr17 | 61456580 | 61456619 | hg38 |
| chr17 | 61456649 | 61456665 | hg38 |
| chr17 | 61456669 | 61456712 | hg38 |
| chr17 | 61456749 | 61456815 | hg38 |
| chr17 | 61457051 | 61457121 | hg38 |
| chr17 | 61457172 | 61457197 | hg38 |
| chr17 | 61457221 | 61457243 | hg38 |
| chr17 | 61457406 | 61457445 | hg38 |
| chr17 | 61457792 | 61457816 | hg38 |
| chr17 | 61966568 | 61966569 | hg38 |
| chr17 | 63237911 | 63237912 | hg38 |
| chr17 | 63701128 | 63701156 | hg38 |
| chr17 | 63831196 | 63831197 | hg38 |
| chr17 | 72115986 | 72116003 | hg38 |
| chr17 | 72116492 | 72116555 | hg38 |
| chr17 | 72116684 | 72116718 | hg38 |
| chr17 | 72116726 | 72116737 | hg38 |
| chr17 | 72117100 | 72117137 | hg38 |
| chr17 | 74671160 | 74671194 | hg38 |
| chr17 | 75753593 | 75753610 | hg38 |
| chr17 | 75753989 | 75754025 | hg38 |
| chr17 | 79815993 | 79816035 | hg38 |
| chr17 | 81648076 | 81648093 | hg38 |
| chr17 | 81648117 | 81648147 | hg38 |
| chr17 | 81648323 | 81648349 | hg38 |
| chr17 | 81648485 | 81648510 | hg38 |
| chr18 | 5196449 | 5196498 | hg38 |
| chr18 | 8608942 | 8608965 | hg38 |
| chr18 | 9749882 | 9749883 | hg38 |
| chr18 | 10032431 | 10032530 | hg38 |
| chr18 | 10032608 | 10032656 | hg38 |
| chr18 | 11148342 | 11148396 | hg38 |
| chr18 | 11148591 | 11148600 | hg38 |
| chr18 | 11148721 | 11148729 | hg38 |
| chr18 | 15198283 | 15198302 | hg38 |
| chr18 | 22165578 | 22165610 | hg38 |
| chr18 | 25159037 | 25159038 | hg38 |
| chr18 | 25349260 | 25349267 | hg38 |
| chr18 | 47247839 | 47247853 | hg38 |
| chr18 | 47248383 | 47248402 | hg38 |
| chr18 | 47248552 | 47248578 | hg38 |
| chr18 | 47248681 | 47248716 | hg38 |
| chr18 | 47248745 | 47248755 | hg38 |
| chr18 | 47248787 | 47248811 | hg38 |
| chr18 | 47248843 | 47248849 | hg38 |
| chr18 | 47248868 | 47248897 | hg38 |
| chr18 | 47248914 | 47248934 | hg38 |
| chr18 | 47249012 | 47249026 | hg38 |
| chr18 | 47249095 | 47249154 | hg38 |
| chr18 | 47250540 | 47250560 | hg38 |
| chr18 | 47250963 | 47250978 | hg38 |
| chr18 | 47261099 | 47261129 | hg38 |
| chr18 | 47262489 | 47262509 | hg38 |
| chr18 | 50967906 | 50968257 | hg38 |
| chr18 | 57354296 | 57354312 | hg38 |
| chr18 | 57428803 | 57428816 | hg38 |
| chr18 | 57648498 | 57648700 | hg38 |
| chr18 | 59274528 | 59274575 | hg38 |
| chr18 | 72869365 | 72869382 | hg38 |
| chr18 | 77087496 | 77087503 | hg38 |
| chr19 | 2291646 | 2291655 | hg38 |
| chr19 | 2291714 | 2291724 | hg38 |
| chr19 | 9498129 | 9498145 | hg38 |
| chr19 | 9498153 | 9498194 | hg38 |
| chr19 | 9498408 | 9498420 | hg38 |
| chr19 | 10514071 | 10514092 | hg38 |
| chr19 | 10514256 | 10514281 | hg38 |
| chr19 | 10514341 | 10514346 | hg38 |
| chr19 | 10514563 | 10514580 | hg38 |
| chr19 | 10514583 | 10514615 | hg38 |
| chr19 | 12194759 | 12194778 | hg38 |
| chr19 | 12195011 | 12195024 | hg38 |
| chr19 | 12555585 | 12555611 | hg38 |
| chr19 | 13099239 | 13099609 | hg38 |
| chr19 | 15122770 | 15122771 | hg38 |
| chr19 | 15233235 | 15233284 | hg38 |
| chr19 | 15233446 | 15233515 | hg38 |
| chr19 | 15428154 | 15428426 | hg38 |
| chr19 | 18870336 | 18870348 | hg38 |
| chr19 | 18913044 | 18913045 | hg38 |
| chr19 | 19540393 | 19540398 | hg38 |
| chr19 | 29529825 | 29529885 | hg38 |
| chr19 | 29529896 | 29529924 | hg38 |
| chr19 | 29529960 | 29529978 | hg38 |
| chr19 | 29530028 | 29530039 | hg38 |
| chr19 | 29530075 | 29530085 | hg38 |
| chr19 | 29530089 | 29530107 | hg38 |
| chr19 | 29530234 | 29530273 | hg38 |
| chr19 | 31351925 | 31351936 | hg38 |
| chr19 | 33088222 | 33088223 | hg38 |
| chr19 | 34905263 | 34905283 | hg38 |
| chr19 | 34905507 | 34905518 | hg38 |
| chr19 | 36421643 | 36421656 | hg38 |
| chr19 | 38256706 | 38256722 | hg38 |
| chr19 | 38395470 | 38395491 | hg38 |
| chr19 | 39264451 | 39264483 | hg38 |
| chr19 | 40613305 | 40614215 | hg38 |
| chr19 | 45152164 | 45152209 | hg38 |
| chr19 | 45152316 | 45152342 | hg38 |
| chr19 | 45152373 | 45152385 | hg38 |
| chr19 | 45152390 | 45152408 | hg38 |
| chr19 | 45152485 | 45152491 | hg38 |
| chr19 | 45385622 | 45385662 | hg38 |
| chr19 | 45395858 | 45395872 | hg38 |
| chr19 | 45396059 | 45396083 | hg38 |
| chr19 | 45396245 | 45396305 | hg38 |
| chr19 | 45396523 | 45396552 | hg38 |
| chr19 | 45396645 | 45396659 | hg38 |
| chr19 | 45396701 | 45396732 | hg38 |
| chr19 | 45396983 | 45397001 | hg38 |
| chr19 | 45815308 | 45815328 | hg38 |
| chr19 | 45815340 | 45815359 | hg38 |
| chr19 | 45815378 | 45815415 | hg38 |
| chr19 | 45815832 | 45815911 | hg38 |
| chr19 | 46413354 | 46413714 | hg38 |
| chr19 | 46493180 | 46493990 | hg38 |
| chr19 | 46494203 | 46494251 | hg38 |
| chr19 | 46494258 | 46494283 | hg38 |
| chr19 | 46494426 | 46494444 | hg38 |
| chr19 | 46494538 | 46494557 | hg38 |
| chr19 | 46494677 | 46494692 | hg38 |
| chr19 | 46494712 | 46494722 | hg38 |
| chr19 | 46494778 | 46494794 | hg38 |
| chr19 | 46648797 | 46648827 | hg38 |
| chr19 | 46648916 | 46648949 | hg38 |
| chr19 | 46648974 | 46648987 | hg38 |
| chr19 | 46648994 | 46649000 | hg38 |
| chr19 | 46649132 | 46649143 | hg38 |
| chr19 | 46649158 | 46649168 | hg38 |
| chr19 | 46649543 | 46649550 | hg38 |
| chr19 | 51719520 | 51719537 | hg38 |
| chr19 | 51719614 | 51719627 | hg38 |
| chr19 | 51719651 | 51719660 | hg38 |
| chr19 | 51719662 | 51719731 | hg38 |
| chr19 | 51719856 | 51719893 | hg38 |
| chr19 | 51949067 | 51949104 | hg38 |
| chr19 | 51949194 | 51949276 | hg38 |
| chr19 | 52453568 | 52453582 | hg38 |
| chr19 | 52453798 | 52453812 | hg38 |
| chr19 | 52535965 | 52535998 | hg38 |
| chr19 | 52536039 | 52536045 | hg38 |
| chr19 | 52536181 | 52536244 | hg38 |
| chr19 | 52536550 | 52536594 | hg38 |
| chr19 | 53254886 | 53254895 | hg38 |
| chr19 | 53978207 | 53978216 | hg38 |
| chr19 | 53978556 | 53978582 | hg38 |
| chr19 | 55081787 | 55081800 | hg38 |
| chr19 | 55082006 | 55082059 | hg38 |
| chr19 | 55082104 | 55082120 | hg38 |
| chr19 | 55082141 | 55082166 | hg38 |
| chr19 | 55082210 | 55082233 | hg38 |
| chr19 | 55082522 | 55082544 | hg38 |
| chr19 | 55086969 | 55087289 | hg38 |
| chr19 | 55161163 | 55161178 | hg38 |
| chr19 | 56671830 | 56671838 | hg38 |
| chr19 | 57106309 | 57106321 | hg38 |
| chr19 | 57106386 | 57106391 | hg38 |
| chr19 | 57106513 | 57106525 | hg38 |
| chr19 | 57106874 | 57106885 | hg38 |
| chr19 | 57107036 | 57107044 | hg38 |
| chr19 | 57583754 | 57583766 | hg38 |
| chr19 | 57583782 | 57583788 | hg38 |
| chr19 | 57708996 | 57709036 | hg38 |
| chr19 | 58033857 | 58033874 | hg38 |
| chr19 | 58440361 | 58440460 | hg38 |
| chr19 | 58440622 | 58440634 | hg38 |
| chr19 | 58440743 | 58440807 | hg38 |
| chr20 | 664334 | 664362 | hg38 |
| chr20 | 763806 | 763807 | hg38 |
| chr20 | 2749681 | 2749709 | hg38 |
| chr20 | 9508928 | 9508958 | hg38 |
| chr20 | 9515871 | 9515890 | hg38 |
| chr20 | 21103694 | 21103707 | hg38 |
| chr20 | 21396013 | 21396029 | hg38 |
| chr20 | 21396155 | 21396185 | hg38 |
| chr20 | 21396195 | 21396221 | hg38 |
| chr20 | 21396359 | 21396386 | hg38 |
| chr20 | 21396396 | 21396413 | hg38 |
| chr20 | 21396417 | 21396428 | hg38 |
| chr20 | 21507786 | 21507803 | hg38 |
| chr20 | 21511069 | 21511106 | hg38 |
| chr20 | 21511314 | 21511373 | hg38 |
| chr20 | 21701389 | 21701445 | hg38 |
| chr20 | 21713890 | 21713909 | hg38 |
| chr20 | 22577018 | 22577035 | hg38 |
| chr20 | 22579893 | 22579926 | hg38 |
| chr20 | 22582389 | 22582420 | hg38 |
| chr20 | 22582744 | 22582757 | hg38 |
| chr20 | 22582791 | 22582805 | hg38 |
| chr20 | 23035385 | 23035394 | hg38 |
| chr20 | 25081534 | 25081546 | hg38 |
| chr20 | 25081705 | 25081741 | hg38 |
| chr20 | 25082232 | 25082251 | hg38 |
| chr20 | 25082624 | 25082631 | hg38 |
| chr20 | 25307996 | 25307997 | hg38 |
| chr20 | 32051626 | 32051663 | hg38 |
| chr20 | 38674686 | 38674705 | hg38 |
| chr20 | 38727589 | 38727661 | hg38 |
| chr20 | 38727777 | 38727805 | hg38 |
| chr20 | 38727927 | 38727932 | hg38 |
| chr20 | 38728096 | 38728124 | hg38 |
| chr20 | 38728629 | 38728651 | hg38 |
| chr20 | 38728658 | 38728664 | hg38 |
| chr20 | 40690779 | 40690805 | hg38 |
| chr20 | 40690806 | 40690840 | hg38 |
| chr20 | 40691093 | 40691128 | hg38 |
| chr20 | 44197739 | 44197740 | hg38 |
| chr20 | 44247939 | 44247962 | hg38 |
| chr20 | 46029744 | 46029769 | hg38 |
| chr20 | 46057572 | 46057580 | hg38 |
| chr20 | 46057977 | 46058008 | hg38 |
| chr20 | 46058153 | 46058194 | hg38 |
| chr20 | 46058212 | 46058243 | hg38 |
| chr20 | 46058484 | 46058505 | hg38 |
| chr20 | 49233645 | 49233646 | hg38 |
| chr20 | 56004874 | 56004884 | hg38 |
| chr20 | 56005395 | 56005406 | hg38 |
| chr20 | 56626005 | 56626023 | hg38 |
| chr20 | 56626071 | 56626084 | hg38 |
| chr20 | 56627083 | 56627105 | hg38 |
| chr20 | 56627533 | 56627546 | hg38 |
| chr20 | 56627646 | 56627676 | hg38 |
| chr20 | 56627956 | 56628038 | hg38 |
| chr20 | 63044219 | 63044255 | hg38 |
| chr20 | 63123533 | 63123560 | hg38 |
| chr20 | 63177097 | 63177125 | hg38 |
| chr20 | 63254853 | 63254904 | hg38 |
| chr20 | 63415034 | 63415104 | hg38 |
| chr21 | 33024787 | 33024794 | hg38 |
| chr21 | 33027256 | 33027274 | hg38 |
| chr21 | 36693601 | 36693642 | hg38 |
| chr21 | 36696347 | 36696361 | hg38 |
| chr21 | 36696813 | 36696829 | hg38 |
| chr21 | 36697248 | 36697264 | hg38 |
| chr21 | 36697338 | 36697363 | hg38 |
| chr21 | 36704931 | 36704959 | hg38 |
| chr21 | 36707704 | 36707719 | hg38 |
| chr21 | 36710814 | 36710828 | hg38 |
| chr22 | 16603179 | 16603189 | hg38 |
| chr22 | 29313365 | 29313370 | hg38 |
| chr22 | 29313371 | 29313387 | hg38 |
| chr22 | 29316049 | 29316068 | hg38 |
| chr22 | 41515521 | 41515522 | hg38 |
| chrX | 91341160 | 91341161 | hg38 |
| chrX | 117469443 | 117469444 | hg38 |
| chrX | 149485920 | 149485921 | hg38 |
| chrY | 7000077 | 7000078 | hg38 |
| chrY | 12914512 | 12914513 | hg38 |
| chrY | 15174113 | 15174114 | hg38 |

1. A method for enriching for a target sequence, the method comprising:
obtaining a mixture of nucleic acid sequences, the mixture comprising a first nucleic acid comprising the target sequence and a second nucleic acid comprising a candidate sequence comprising one or more variant sequences;
providing an enrichment probe that binds to the target sequence of the first nucleic acid, or a portion thereof;
providing a plurality of blocker sequences, each blocker probe comprising a sequence complementary to at least one variant sequence of the candidate sequence; and
selectively enriching for the target sequence using the enrichment probe without enriching for the one or more variant sequences of the candidate sequence.

2. The method of claim 1, wherein the target sequence comprises a sequence comprising one or more methylated CpG sites, or a sequence derived from the sequence comprising one or more methylated CpG sites.

3. The method of claim 2, wherein the target sequence comprises a sequence comprising at least five consecutive methylated CpG sites, or a sequence derived from the sequence comprising at least five consecutive methylated CpG sites.

4. The method of claim 1, wherein the one or more variant sequences of the candidate sequence comprise one or more unmethylated CpG sites.

5. The method of claim 4, wherein the one or more variant sequences comprise at least five consecutive non-methylated CpG sites.

6. The method of claim 4, wherein each variant sequence of the one or more variant sequences comprises a single unmethylated CpG site.

7. The method of claim 4, wherein the candidate sequence comprises five or more variant sequences comprising five or more consecutively unmethylated CpG sites.

8. (canceled)

9. The method of claim 1, wherein the candidate sequence differs from the target sequence by one or more nucleotides based on differential methylation of a corresponding one or more CpG sites.

10. The method of claim 1, wherein the enrichment probe is between about 50 nucleotide bases and about 150 nucleotide bases.

11. The method of claim 1, wherein the enrichment probe is between about 60 nucleotide bases and about 80 nucleotide bases.

12. The method of claim 1, wherein the enrichment probe is about 70 nucleotide bases.

13. The method of claim 1, wherein the enrichment probe is between about 80 nucleotide bases and about 120 nucleotide bases.

14. The method of claim 1, wherein the enrichment probe is about 100 nucleotide bases.

15. The method of claim 1, wherein each of the plurality of blocker sequences are between about 10 and about 20 nucleotide bases in length.

16. The method of claim 1, wherein each of the plurality of blocker sequences are between about 15 and about 18 nucleotide bases in length.

17. The method of claim 1, wherein each of the plurality of blocker sequences are about 17 nucleotide bases in length.

18. The method of claim 1, wherein the enrichment probe is between about 4 and about 6 times longer in length in comparison to an average length of the plurality of blocker sequences or in comparison to each of the plurality of blocker sequences.

19. (canceled)

20. The method of claim 1, wherein selectively enriching for the target sequence using the enrichment probe without enriching for the one or more variant sequences comprises:
hybridizing the enrichment probe to the target sequence, or a portion thereof;
hybridizing the plurality of blocker sequences to the one or more variant sequences of the candidate sequence; and
enriching for the hybridized enrichment probe while the one or more hybridized blocker probes prevents enrichment for the one or more variant sequences.

21. The method of claim 20, wherein enriching for the hybridized enrichment probe while the one or more hybridized blocker probes prevents enrichment for the one or more variant sequences comprises:
binding a streptavidin bead to a biotin group of the enrichment probe; and
washing and removing unbound nucleic acids, thereby enriching for the target sequence.

22. The method of claim 21, wherein the unbound nucleic acids comprise one or more blocker sequences and/or candidate sequences.
